(19) United States
(12) Patent Application Publication
(10) Pub. No.: US 2023/0277624 A1
(43) Pub. Date: Sep. 7, 2023

(54) METHODS OF DETERMINING RESPONSIVENESS TO CANCER IMMUNOTHERAPY

(71) Applicant: OncoSec Medical Incorporated, Pennington, NJ (US)

(72) Inventor: Christopher G. TWITTY, San Diego, CA (US)

(21) Appl. No.: 18/011,501

(22) PCT Filed: Jun. 18, 2021
(86) PCT No.: PCT/US2021/038005

§ 371 Date: Dec. 19, 2022

Related U.S. Application Data
(60) Provisional application No. 63/041,493, filed on Jun. 19, 2020.

(51) Int. Cl. A61K 38/19; C12N 15/85; G01N 33/574; A61K 39/395; A61K 41/00; A61K 38/20; A61P 35/00

(52) U.S. Cl. CPC A61K 38/195; C12N 15/85; G01N 33/57492; A61K 39/3955; A61K 41/0047; A61K 38/208; A61K 38/2086; A61P 35/00; C12N 2800/107

(57) ABSTRACT

Described are compositions comprising nucleic acids encoding CD3-half-BiTE, CXCL9, CTLA-4 scFv, and IL-12for use in treating cancer. Methods of analyzing CXCR3 expression in a tumor to identify subjects likely to respond to the compositions are also described.

### CROSS-REFERENCE TO RELATED APPLICATIONS

This application claims the benefit of U.S. Provisional Application No. 63/041,493, filed Jun. 19, 2020, which is incorporated herein by reference.

### SEQUENCE LISTING

The Sequence Listing written in file 560207_SeqListing_CXCR3_ST25 is 144 kilobytes in size, was created Jun. 18, 2021, and is hereby incorporated by reference.

### BACKGROUND

Cancer immunoediting is responsible for eliminating tumors and sculpting the immunogenic phenotypes of tumors that eventually form in immunocompetent hosts following tumor escape from immune destruction. Immune system-tumor interactions are postulated to occur in three continuous phases: elimination, equilibrium, and escape. Elimination entails the destruction of tumor cells by T lymphocytes. In equilibrium, a population of immune-resistant tumor cells appears. During escape, the tumor has developed strategies to evade immune detection or destruction. Escape may occur through loss or ineffective presentation of tumor antigens, secretion of inhibitory cytokines, or downregulation of major histocompatibility complex molecules.

Cancer immunotherapy aims to elicit successful T-cell response that leads to cancer regression. Various efforts have been made to activate effector T-cell responses, such as though presentation of tumor antigen by antigen presenting cells (APCs), priming T cells to successfully target and infiltrate tumors, and enhancing infiltrating T cells to bind to the MHCI-peptide complex to activate a cytotoxic T-cell response.

Studies have shown a survival benefit associated with the presence of tumor infiltrating lymphocytes (TILs) There is evidence that immunostimulatory cytokines, such as IL-12, can increase the immune cell infiltrate in solid tumors. However, systemic administration of IL-12 has a narrow therapeutic index and is often accompanied by unacceptable levels of adverse events. The limitations of systemic administration of IL-12 can be overcome by therapies that result in local expression of IL-12, such as intratumoral electroporation of plasmid encoding IL-12.

Identification of patients likely to response to cancer immunotherapies would be useful in targeting therapies to patients who are most likely to benefit from the therapies. In addition, it would be helpful to identify therapies that convert non-responding patients to responding patients.

While IL-12 can increase the number of TILs, there remains a need to increase the presence and number of tumor-specific T cells in a tumor. CD3 (cluster of differentiation 3) T cell co-receptor helps to activate both the cytotoxic T cell (CD8⁺ naive T cells) and also T helper cells (CD4⁺ naive T cells). Because of its role in activating T cell response, anti-CD3 antibodies have been explored for use as immunosuppressant therapies. Bispecific antibodies, including Bi-specific T-cell engagers (BiTEs), targeting CD3 and a cancer antigen (tumor marker) have been developed to target T cells to cancer cells.

### SUMMARY

Described are methods of treating cancer in a subject comprising: (a) administering at least one dose of a checkpoint inhibitor and/or an immunostimulatory cytokine to the subject; (b) obtaining a tumor sample from the subject; (c) measuring CXCR3 expression in the tumor sample; (d) determining whether CXCR3 expression is increased in the tumor sample relative to CXCR3 expression in a predetermined control; and (e) if CXCR3 expression is increased in the tumor sample relative to CXCR3 expression in the predetermined control, then administering at least one additional dose of the checkpoint inhibitor and/or an immunostimulatory cytokine to the subject, or if CXCR3 expression is not increased in the tumor sample relative to CXCR3 expression in the predetermined control, then administering at least one pharmaceutically effective dose of CXCL9 and/or CD3 half-BiTE and at least one additional dose of the checkpoint inhibitor and/or an immunostimulatory cytokine to the subject. In some embodiments, the CXCL9 and/or CD3 half-BiTE is administered in combination with IL-12. The CXCL9 and/or CD3 half-BiTE can be administered prior to, concurrently with, or subsequent to administration of IL-12. The CXCL9, CD3 half-BiTE, and/or IL-12 can be administered by intratumoral electroporation (IT-EP) of a nucleic acid encoding the CXCL9, CD3 half-BiTE, and/or IL-12. In some embodiments, the checkpoint inhibitor therapy comprises anti-PD-1/anti-PD-L1 therapy. The checkpoint inhibitor therapy may be administered systemically. In some embodiments, the immunostimulatory cytokine comprises IL-12 or a nucleic acid encoding IL-12. In some embodiments, the at least one dose of a checkpoint inhibitor and/or an immunostimulatory cytokine comprises a dose that would be typically be considered pharmaceutically effective in responsive subjects.

Described are methods of treating cancer in a subject comprising: (a) administering at least one dose of a checkpoint inhibitor and/or an immunostimulatory cytokine to the subject; (b) measuring a level of CXCR3 in a tumor sample obtained from the subject after the step of administering the checkpoint inhibitor and/or the immunostimulatory cytokine; and (c) administering to the subject at least one pharmaceutically effective dose of CXCL9 and/or CD3 half-BiTE if the level of CXCR3 in the tumor sample is not increased relative to the level of CXCR3 in a predetermined control. In some embodiments, the CXCL9 and/or CD3 half-BiTE is administered in combination with IL-12. The CXCL9 and/or CD3 half-BiTE can be administered prior to, concurrently with, or subsequent to administration of IL-12. The CXCL9, CD3 half-BiTE, and/or IL-12 can be administered by intratumoral electroporation (IT-EP) of a nucleic acid encoding the CXCL9, CD3 half-BiTE, and/or IL-12. In some embodiments, the checkpoint inhibitor therapy comprises anti-PD-1/anti-PD-L1 therapy. The checkpoint inhibitor therapy may be administered systemically. In some embodiments, the immunostimulatory cytokine comprises IL-12 or a nucleic acid encoding IL-12. In some embodiments, the at least one dose of a checkpoint inhibitor and/or an immunostimulatory cytokine comprises a dose that would be typically be considered pharmaceutically effective in responsive subjects.

Described are methods of determining whether a subject having cancer is at risk of not responding to checkpoint inhibitor and/or immunostimulatory cytokine therapy. The methods comprise: measuring a level of CXCR3 in a tumor sample obtained from the subject that has been administered at least one dose of a checkpoint inhibitor and/or an immunostimulatory cytokine, wherein the level of CXCR3 in the tumor sample less than a predetermined control indicates the subject is at risk of not responding to the checkpoint inhibitor and/or immunostimulatory cytokine therapy. In some embodiments, a subject determined to be at risk of not responding to checkpoint inhibitor and/or immunostimulatory cytokine therapy is administered at least one pharmaceutically effective dose of CXCL9 and/or CD-3 half-BiTE. In some embodiments, the CXCL9 and/or CD3 half-BiTE is administered in combination with IL-12. The CXCL9 and/or CD3 half-BiTE can be administered prior to, concurrently with, or subsequent to administration of IL-12. The CXCL9, CD3 half-BiTE, and/or IL-12 can be administered by intratumoral electroporation (IT-EP) of a nucleic acid encoding the CXCL9, CD3 half-BiTE, and/or IL-12. In some embodiments, the checkpoint inhibitor therapy comprises anti-PD-1/anti-PD-L1 therapy. The checkpoint inhibitor therapy may be administered systemically. In some embodiments, the immunostimulatory cytokine therapy comprises intratumoral electroporation of a nucleic acid encoding IL-12. In some embodiments, the at least one dose of a checkpoint inhibitor and/or an immunostimulatory cytokine comprises a dose that would be typically be considered pharmaceutically effective in responsive subjects.

Described are methods of treating a patient with cancer comprising: (a) obtaining a tumor sample from the patient, (b) measuring a level of CXCR3 expression in the tumor sample, (c) correlating the level of CXCR3 expression in the tumor sample with a reference level obtained from a predetermined control or standard derived from a population of known responders and/or known non-responders to determine whether the patient is at risk of progressing on checkpoint inhibitor therapy, and (d) if the expression level is greater than the reference level, then administering at least one dose of a checkpoint inhibitor and/or an immunostimulatory cytokine, or if the expression level is less than the reference level then administering to the patient at least one pharmaceutically effective dose of CXCL9 and/or CD3 half-BiTE and at least one dose of the checkpoint inhibitor and/or the immunostimulatory cytokine. In some embodiments, the CXCL9 and/or CD3 half-BiTE is administered in combination with IL-12. The CXCL9 and/or CD3 half-BiTE can be administered prior to, concurrently with, or subsequent to administration of IL-12. The CXCL9, CD3 half-BiTE, and/or IL-12 can be administered by intratumoral electroporation (IT-EP) of a nucleic acid encoding the CXCL9, CD3 half-BiTE, and/or IL-12. In some embodiments, the checkpoint inhibitor comprises an anti-PD-1/anti-PD-L1 antibody. The checkpoint inhibitor therapy may be administered systemically. In some embodiments, the immunostimulatory cytokine comprises IL-12 or a nucleic acid encoding IL-12. In some embodiments, the at least one dose of a checkpoint inhibitor and/or an immunostimulatory cytokine comprises a dose that would be typically be considered pharmaceutically effective in responsive subjects.

Described are methods of treating cancer in a subject comprising: (a) obtaining a tumor sample from the subject; (b) measuring CXCR3 expression in the tumor sample; (c) determining whether CXCR3 expression is increased in the tumor sample relative to CXCR3 expression in a predetermined control; and (d) if CXCR3 expression is increased in the tumor sample relative to CXCR3 expression in the predetermined control, then administering at least one dose of the checkpoint inhibitor and/or an immunostimulatory cytokine to the subject, or if CXCR3 expression is not increased in the tumor sample relative to CXCR3 expression in the predetermined control, then administering at least one pharmaceutically effective dose of CXCL9 and/or CD3 half-BiTE and at least one dose of a checkpoint inhibitor and/or an immunostimulatory cytokine to the subject. In some embodiments, the CXCL9 and/or CD3 half-BiTE is administered in combination with IL-12. The CXCL9 and/or CD3 half-BiTE can be administered prior to, concurrently with, or subsequent to administration of IL-12. The CXCL9, CD3 half-BiTE, and/or IL-12 can be administered by intratumoral electroporation (IT-EP) of a nucleic acid encoding the CXCL9, CD3 half-BiTE, and/or IL-12. In some embodiments, the checkpoint inhibitor comprises anti-PD-1/anti-PD-L1 therapy. The checkpoint inhibitor therapy may be administered systemically. In some embodiments, administration of the immunostimulatory cytokine therapy comprises intratumoral electroporation of a nucleic acid encoding IL-12. In some embodiments, the predetermined control comprises a standard derived from a population of known responders and/or known non-responders to checkpoint inhibitor and/or an immunostimulatory cytokine therapy. In some embodiments, the predetermine control comprises a tumor sample obtained prior to administration of one or more therapies to the subject. The prior therapy can be, but is not limited to, IL-12 therapy, checkpoint inhibitor therapy, or a combination thereof. In some embodiments, the at least one dose of a checkpoint inhibitor and/or an immunostimulatory cytokine comprises a dose that would be typically be considered pharmaceutically effective in responsive subjects.

Described are methods of identifying a subject with cancer at risk of not responding to checkpoint inhibitor and/or immunostimulatory cytokine therapy comprising: measuring a level of CXCR3 in a tumor sample obtained from the subject, wherein the level of CXCR3 in the tumor sample less than a predetermined control indicates the subject is at risk of not responding to the checkpoint inhibitor and/or immunostimulatory cytokine therapy. Measuring a level of CXCR3 expression in the tumor sample can comprise: measuring CXCR3 mRNA in the tumor sample, measuring CXCR3 protein in the tumor sample, or measuring a number of CXCR3⁺ T cells in the tumor sample. In some embodiments, the predetermined control comprises a standard derived from a population of known responders and/or known non-responders to checkpoint inhibitor and/or an immunostimulatory cytokine therapy. In some embodiments, the predetermine control comprises a tumor sample obtained prior to administration of one or more therapies to the subject. The prior therapy can be, but is not limited to IL-12 therapy, checkpoint inhibitor therapy, or a combination thereof.

Described are expression cassettes (e.g., nucleic acids) encoding CXCL9, CXCL9 plus IL-12, anti-CTLA-4 scFv, anti-CTLA-4 scFv plus IL-12, CD3 half-BiTE and CD3 half-BiTE plus IL-12. The describe expression cassettes are useful in the treatment of cancer. In some embodiments, the expression cassettes are useful in treating cancer in a subject that has failed to respond to at least one course of anti-PD-1/anti-PD-L1 therapy, is predicted to be at risk of not responding to anti-PD-1/anti-PD-L1 therapy, is progressing on anti-PD-1/anti-PD-L1 therapy, or has progressed on anti-PD-1/anti-PD-L1 therapy. Methods of using the described expression cassettes to treat tumors, including cancers and metastatic cancers, are also described. The described expression cassettes, when delivered to a tumor, such as by electroporation, result in local tumor expression of the encoded proteins, leading to T cell recruitment and anti-tumor activity. In some embodiments, the methods also result in abscopal effects, i.e., regression of one or more untreated tumors. In some embodiments, regression includes debulking of a solid tumor.

Expression cassettes encoding CXCL9 are described. In some embodiments, an expression cassette encoding CXCL9 further encodes IL-12. The described CXCL9 expression cassettes can be delivered intratumorally, peritumorally, into a lymph node, intradermally, and/or intramuscularly. In some embodiments, the CXCL9 and IL12 coding sequences are expressed on a multicistronic expression cassette from a single promoter and separated by an IRES or 2A translation modification element. In some embodiments, the 2A element is a P2A element. IL-12 is a heterodimeric cytokine having both IL-12A (p35) and IL-12B (p40) subunits. The encoded IL-12 can comprise a fusion construct encoding an IL-12 p35-IL-12 p40 fusion protein (IL12 p70). In some embodiments, the IL-12 p35 and p40 coding sequences are expressed from a multicistronic expression cassette from a single promoter and separated by an IRES or 2A element. In some embodiments, the 2A element is a P2A element. In some embodiments, multicistronic expression cassettes are described, comprising CXCL9, IL12 p35, and IL-12 p40 coding regions separated by IRES or 2A elements. In some embodiments, the 2A element is a P2A element.

Expression cassettes encoding anti-CTLA-4 scFvs are described. An anti-CTLA-4 scFv comprises an anti-CTLA-4 single-chain variable fragment. The described anti-CTLA-4 scFv expression cassettes can be delivered intratumorally, peritumorally, into a lymph node, intradermally, and/or intramuscularly. The lymph node can be a draining lymph node. An anti-CTLA-4 scFv expression cassette can also be delivered in a peritumoral region between the tumor and the draining lymph node. For each of intratumoral, peritumoral, lymph node, intradermal, and/or intramuscular delivery of an anti-CTLA-4 scFv expression cassette, the delivery can be facilitated by electroporation. Direct expression of an anti-CTLA-4 scFv expression cassette can result in fewer side effects and/or toxicity when compared to systemic administration of anti-CTLA-4 antibodies. The described anti-CTLA-4 scFv expression cassettes facilitate delivery of local yet efficacious dose of anti-CTLA-4.

CD3 half-BiTEs and expression cassettes encoding CD3 half-BiTEs are described. CD3 half-BiTEs comprise anti-CD3 single-chain variable fragment (scFv) fused to a transmembrane domain (TM). In some embodiments, an expression cassette encoding a CD3 half-BiTE further encodes a signal peptide. The encoded signal peptide can be operably linked to the 5′ end of the anti-CD3 single-chain variable fragment coding sequence. In some embodiments, an expression cassette encoding a CD3 half-BiTE further encodes IL-12. The described CD3 half-BiTE expression cassettes can be delivered intratumorally, peritumorally, into a lymph node, intradermally, and/or intramuscularly. In some embodiments, the CD3 half-BiTE and IL12 coding sequences are expressed on a multicistronic expression cassette from a single promoter and separated by an IRES or 2A translation modification element. In some embodiments, the 2A element is a P2A element. IL-12 is heterodimeric cytokine having both IL-12A (p35) and IL-12B (p40) subunits. The encoded IL-12 can contain a fusion construct encoding an IL-12 p35-IL-12 p40 fusion protein (IL12 p70). In some embodiments, the IL-12 p35 and p40 coding sequences are expressed from a multicistronic expression cassette from a single promoter and separated by an IRES or 2A translation modification element. In some embodiments, the 2A element is a P2A element. In some embodiments, multicistronic expression cassettes are described, comprising a CD3 half-BiTE, IL12 p35, and IL-12 p40 coding regions separated by IRES or 2A translation modification elements. In some embodiments, the 2A element is a P2A element.

Described are methods of treating a cancer comprising administering to a subject, by intratumoral electroporation (IT-EP), a composition comprising a therapeutically effective amount one or more of the described expression cassettes. The composition is injected into a tumor, tumor microenvironment, and/or tumor margin tissue and electroporation therapy is applied to the tumor, tumor microenvironment, and/or tumor margin tissue. The electroporation therapy may be applied by any suitable electroporation system known in the art. In some embodiments, the electroporation is at a field strength of about 60 V/cm to about 1500 V/cm, and a duration of about 10 microseconds to about 20 milliseconds. In some embodiments, the electroporation incorporates Electrochemical Impedance Spectroscopy (EIS). The subject can be a mammal. The mammal can be, but is not limited to, a human, canine, feline, or equine.

In some embodiments, the methods further comprise administering to the subject a therapeutically effect amount of an immunostimulatory cytokine. The immunostimulatory cytokine can be an expression cassette encoding the immunostimulatory cytokine delivered by IT-EP. The immunostimulatory cytokine can be, but is not limited to, IL-12. The immunostimulatory cytokine can be delivered prior to, subsequent to, or concurrent with one or more of the described CXCL9, CTLA-4 scFv and CD3 half-BiTE expression cassettes.

In some embodiments, the methods further comprise administration of one or more additional therapies. The one or more additional therapies can be, but are not limited to, immune checkpoint therapy. Immune checkpoint therapy can be, but is not limited to, administration of one or more immune checkpoint inhibitors. “Immune checkpoint” molecules refer to a group of immune cell surface receptor/ligands which induce T cell dysfunction or apoptosis. These immune inhibitory targets attenuate excessive immune reactions and ensure self-tolerance. Tumor cells harness the suppressive effects of these checkpoint molecules. Immune checkpoint target molecules include, but are not limited to, Cytotoxic T Lymphocyte Antigen-4 (CTLA-4), Programmed Death 1 (PD-1), Programmed Death Ligand 1 (PD-L1), Lymphocyte Activation Gene-3 (LAG-3), T cell Immunoglobulin Mucin-3 (TIM3), Killer Cell Immunoglobulin-like Receptor (MR), B- and T-Lymphocyte Attenuator (BTLA), Adenosine A2a Receptor (A2aR), and Herpes Virus Entry Mediator (HVEM). “Immune checkpoint inhibitors” include molecules that prevent immune suppression by blocking the effects of immune checkpoint molecules. Checkpoint inhibitors include, but are not limited to, antibodies and antibody fragments, nanobodies, diabodies, soluble binding partners of checkpoint molecules, small molecule therapeutics, and peptide antagonists. An immune checkpoint inhibitor can be, but is not limited to, a PD-1 and/or PD-L1 antagonist. A PD-1 and/or PD-L1 antagonist can be, but is not limited to, an anti-PD-1 or anti-PD-L1 antibody. Anti-PD-1/anti-PD-L1 antibodies include, but are not limited to, nivolumab, pembrolizumab, pidilizumab, and atezolizumab. Immune checkpoint (checkpoint inhibitor) therapy may be administered systemically.

Described are methods of treating a tumor in a subject comprising: administering at least one treatment cycle to the subject, the cycle comprising: administering to the tumor, by IT-EP, a composition comprising a therapeutically effective amount of one or more of the described CXCL9, CXCL9 plus IL-12 (i.e., IL12˜CXCL9), anti-CTLA-4 scFv, anti-CTLA-4 scFv plus IL-12, CD3 half-BiTE, or CD3 half-BiTE plus IL-12 (i.e., CD3 half-BiTE˜IL12) expression cassettes. In some embodiments, the cycle is a three week cycle. In some embodiments, the cycle is a four, five, or six week cycle. The composition can be administered by IT-EP on 1, 2, 3, 4, 5, or 6 days of a cycle. In some embodiments, the composition is administered by IT-EP on day 1 of each cycle. In some embodiments, the composition administered by IT-EP on days 1 and 5±2 of each cycle. In some embodiments, the composition is administered by IT-EP on days 1 and 8±2 of each cycle. In some embodiments, the composition is administered by IT-EP on days 1, 5±2, and 8±2 of each cycle. The cycles can be repeated as often as is necessary to treat the subject. In some embodiments, a cycle further comprises administration of an additional therapeutic. The additional therapeutic can be, but is not limited to, an immune checkpoint therapy. In some embodiments, the immune checkpoint therapy is administered to the subject on day 1, 2, or 3 of the cycle.

In some embodiments, a subject is treated with one of more cycles of IT-EP therapy with one or more of the described expression cassettes. Any of the above cycles can be repeated in subsequent cycles. The subsequent cycles can be consecutive cycles or alternating cycles. Alternating cycles can have one or more intervening cycles of no therapy or alternative therapy (e.g., immune checkpoint therapy). For example, any of the described expression cassettes can be administered on days 1, 5±2, and 8±2 of alternating cycles (e.g., cycles 1, 3, 5, etc. as needed) and an alternative therapy can be administered, e.g., on day 1, 2, or 3, of consecutive cycles (e.g., cycles 1, 2, 3, 4, 5, etc. as needed).

In some embodiments, a subject is administered alternating cycles of IT-EP of any of the described CXCL9, CTLA-4 scFv, and/or CD3 half-BiTE expression cassettes, with or without immune checkpoint inhibitor therapy, and immune checkpoint inhibitor therapy. In other words, a subject can be administered, by IT-EP, a composition comprising a therapeutically effective amount of one or more of the described CXCL9, CXCL9 plus IL-12, anti-CTLA-4 scFv, anti-CTLA-4 scFv plus IL-12, CD3 half-BiTE, or CD3 half-BiTE plus IL-12 expression cassettes and optionally administered immune checkpoint inhibitor therapy on odd numbered cycles (cycles 1, 3, etc.) and administered immune checkpoint inhibitor therapy on even numbered cycles (cycles 2, 4, etc.). Alternatively, a patient can be administered immune checkpoint inhibitor therapy on odd numbered cycles (cycles 1, 3, etc.) and administered, by IT-EP, a composition comprising a therapeutically effective amount of one or more of the described CXCL9, CXCL9 plus IL-12, anti-CTLA-4 scFv, anti-CTLA-4 scFv plus IL-12, CD3 half-BiTE, or CD3 half-BiTE plus IL-12 expression cassettes and optionally administered immune checkpoint inhibitor therapy on even numbered cycles (cycles 2, 4, etc).

The expression cassettes and methods can be used to treat a subject having advanced, metastatic, and/or treatment refractory tumor. A treatment refractory tumor can be, but is not limited to, an immune checkpoint inhibitor refractory tumor, a hormone refractory tumor, a radiation refractory tumor, or a chemotherapy refractory tumor. In some embodiments, the subject has failed to respond to at least one course of immune checkpoint inhibitor therapy. In some embodiments, the subject is progressing on or has progressed on one or more anti-cancer therapies, such as, but not limited to, checkpoint inhibitor therapy. In some embodiments, the subject has failed to respond to at least one course of anti-PD-1/anti-PD-L1 therapy, is predicted to be at risk of not responding to anti-PD-1/anti-PD-L1 therapy, is progressing on anti-PD-1/anti-PD-L1 therapy, or has progressed on anti-PD-1/anti-PD-L1 therapy.

The expression cassettes and methods can be used to treat subjects having tumors predicted to be refractory to or not respond to one or more anti-cancer therapies. In some embodiments, the subject has low tumor infiltrating lymphocytes, low partially cytotoxic lymphocytes, or exhausted T cells. In some embodiments, the described expression cassettes and methods are used to treat a subject having a CXCR3 level in a tumor sample obtained from the subject that is not increased in response to checkpoint inhibitor and/or an immunostimulatory cytokine therapy. In some embodiments, the described expression cassettes and methods are used to treat a subject having a CXCR3 level in a tumor sample obtained from the subject that is not increased in response to anti-PD-1/anti-PD-L1 and/or an IL-12 therapy. In some embodiments, the described expression cassettes and methods are used to treat a subject having a CXCR3 level in a tumor sample obtained from the subject is less than a standard derived from a population of known responders and/or known non-responders. In some embodiments, the subject has advanced on one or more prior cancer therapies.

### BRIEF DESCRIPTION OF THE DRAWINGS

FIG. 1A. Illustrations of the expression constructs for mCXCL9˜mCherry (mCXCL9-P2A-mCherry), mCXCL9, mIL12-2A (mIL-12 p35-P2A-mIL-12 p40), mIL12˜mCXCL9 (mIL-12 p35-P2A-mIL-12 p40-P2A-mCXCL9).

FIG. 1B. Illustrations of expression constructs for hCXCL9, hIL12-2A (hIL-12 p35-P2A-hIL-12 p40), hIL12-hCXCL9 (hIL-12 p35-P2A-hIL-12 p40-P2A-hCXCL9).

FIG. 2. Graphs illustrating (A) mIL12p70 protein expression, and (B) mCXCL9 protein expression in HEK293 cells following transfection with mIL12-2A, mCXCL9, and mIL12˜mCXCL9 expression vectors.

FIG. 3. Graph illustrating dose-response to mIL-12p70 from transiently transfected HEK293 cells with mouse IL-12 or mouse IL-12-CXC constructs. Both constructs encode biologically active IL-12.

FIG. 4A. Graph illustrating transfection-derived mouse CXCL9 induced chemotaxis of SIINFEKL-pulsed (24 hr @ 1 μg/mL, 72 hr recovery) OT-I splenocytes through polycarbonate membranes with 5.0-micron pores (Costar 3421). Migration index is defined as the number of observed chemotactic cells after 2.5 hours at 37° C., normalized to the number of cells that passively migrated through the membrane in the OptiMEM negative control. Abrogation of chemotaxis was observed with the pre-incubation of anti-mCXCL9 neutralizing monoclonal antibody (BioXCell BE0309).

FIG. 4B. Graph illustrating transfection-derived (HEK293) human CXCL9-induced chemotaxis of SIINFEKL-pulsed (24 hr @ 1 μg/mL, 72 hr recovery) OT-I splenocytes through polycarbonate membranes with 5.0-micron pores (Costar 3421). Migration index is defined as the number of observed chemotactic cells after 2 hours at 37° C., normalized to the number of cells that passively migrated through the membrane towards the OptiMEM negative control.

FIG. 4C. Graph illustrating transfection-derived (HEK293) human CXCL9-induced chemotaxis of human peripheral mononuclear cells (thawed from cryopreservation, rested for 24 hr in X-VIVO15 medium) through polycarbonate membranes with 5.0-micron pores (Costar 3421). Migration index is defined as the number of observed chemotactic cells after 2 hours at 37° C., normalized to the number of cells that passively migrated through the membrane towards the OptiMEM negative control.

FIG. 5. Graph illustrating intratumoral expression of mCXCL9 using ELISA for mCXCL9 (DuoSet ELISA DY392) 48 hrs post-electroporation in tumor lysates from mice bearing CT26 tumors (n=3; *P<0.05; T test with Welch correction).

FIG. 6. Graphs illustrating Kaplan-Meir curves in untreated mice and mice treated with control vector, IT-EP IL12-2A alone, or IT-EP IL12-2A in combination with IT-EP CXCL9 (**P<0.005; log-rank (Mantel-Cox) test).

FIG. 7. Graphs illustrating (A) decreased tumor volume, and (B) decreased contralateral (untreated) tumor volume, in tumor bearing mice treated with IT-EP therapy with mIL12-2A plus mCXCL9 compared to IL-12 therapy alone on control plasmid.

FIG. 8. Flow cytometric analysis of splenocytes from mice treated with IT-EP pUMCV3 or IL12-2A on day 0 and IT-EP pUMVC3 or mCXCL9 on days 4 and 7

FIG. 9. Graph illustrating fold increase in the number of AH1+ CD8+ T cells in mice tumors treated with control vector (pUMVC3), IT-EP IL12 (IL-12 p35-P2A-IL-12 p40), or IT-EP IL12 plus IT-EP CXCL9. N=2 independent experiments with 3-5 animals/group; *P<0.05, **P<0.005; One way ANOVA.

FIG. 10. Graphs illustrating (A) hIL-12 protein expression in HEK293 cells transfected with hIL12-2A and hIL12˜hCXCL9 expression vectors and (B) hCXCL9 protein expression in HEK293 cells transfected with hCXCL9 and hIL12˜hCXCL9 expression vectors.

FIG. 11. Graph illustrating activation of STAT4 pathway in HEK-Blue IL-12 cells using recombinant human IL-12 (rhIL12, positive control), or hIL12 produced from cells expressing a hIL12-2A expression vector.

FIG. 12A. Illustrations of the mouse CD3 half-BiTE expression cassettes for HA-2C11-Myc scFv, HA-2C11 scFv, 2C11 scFv, and 2C11 scFv˜hIL12.

FIG. 12B. Illustrations of human CD3 half-BiTE expression cassettes for HA-OKT3-Myc scFv, HA-OKT3 scFv, OKT3 scFv, HA-OKT3 scFv˜hIL12, and OKT3 scFv˜hIL12.

FIG. 13. Western blots showing: (A) expression of anti-CD3 scFv in HEK293 cells transfected with HA-OKT3 scFv and HA-2C11 scFv CD3 half-BiTE expression vectors, and (B) expression of CD3 half-BiTE in B16-F10 cells transfected with HA-2C11 scFv and HA-2C11 scFv˜mIL12 expression vectors.

FIG. 14A-C. Flow cytometry showing the surface expression of anti-CD3 scFv in HEK 293 cells transfected with HA-OKT3 scFv and HA-OKT3 scFv˜hIL12 expression vectors.

FIG. 14D-E. (D) Flow cytometry showing the surface expression of anti-CD3 scFv in B16-F10 cells transfected with HA-2C11 scFv and HA-2C11 scFv˜mIL12 expression vectors. (E) Graph illustrating IL12p70 expression in B16-F10 cells following transfection with mIL12-2A, HA-2C11 scFv˜mIL12 expression vector.

FIG. 15. Graph illustrating IL12p70 expression in HEK293 cells following transfection with hIL12-2A, HA-OKT3 scFv˜hIL12, and OKT3 scFv˜hIL12 expression vectors.

FIG. 16A-B. (A) Western blot showing expression of CD3 scFv in B16F10 melanoma or 4T1 breast cancer cells in vivo following intratumoral electroporation of HA-2C11 scFv. (B) Flow analysis of surface expression of CD3 scFv on 4T1 breast cancer cells in vivo following intratumoral electroporation of HA-2C11 scFv.

FIG. 16C. Graph illustrating IL12p70 expression in B16-F10 cells following intratumoral electroporation of mIL12-2A and HA-2C11 scFv˜mIL12 expression vectors.

FIG. 17. Graph illustrating induction of IFNγ expression following co-culture of naïve mouse splenocytes with B16F10 cells transfected in vitro with control vector (EV control), 2C11 scFv expression vector with or without recombinant mouse IL12, or with plate bound anti-CD3 (positive control).

FIG. 18. Graphs illustrating FACS analyses of proliferation of CFSE labeled CD3+CD45+ T cells following co-culture of naïve mouse splenocytes with B16F10 cells transfected in vitro with control vector (Tfx control), 2C11 scFV expression vector with or without recombinant mouse IL12, or with plate bound anti-CD3 (positive control).

FIG. 19. Graph illustrating in vivo OT-1 and polyclonal T cell proliferation in DLN in B16-OVA tumor model mice treated with 2C11 scFv IT-EP or negative control.

FIG. 20. Graphs illustrating an increased CD8+ T cells in CD45.1+ live cells in TILs in B16-OVA tumor model mice treated with 2C11 scFv IT-EP or negative control.

FIG. 21. Graph illustrating an increased antigen specific (SIINFEKL+) CD8+ T cells in TILs in B16-OVA tumor model mice treated with 2C11 scFv IT-EP or negative control.

FIG. 22. FACS analysis of scan CFSE cells displaying (Hi) or not displaying (Lo) OVA₂₅₇₋₂₆₄ peptide showing increase lysis of OVA₂₅₇₋₂₆₄ peptide-displaying CFSE cells in B16-OVA tumor containing mice treated with 2C11 scFv IT-EP compare with negative transfected control.

FIG. 23. Graph illustrating increase in lysis of adoptive transferred OVA₂₅₇₋₂₆₄-displaying CFSE cells in B16-OVA tumor containing mice treated with IT-EP CD3 half-BiTE. Increased T cell killing ability observed in both spleen and draining lymph node.

FIG. 24. FACS analysis of CFSE cells showing increase tumor-specific killing of OVA expressing cells in mice treated with IT-EP CD3 half-BiTE.

FIG. 25. Graph illustrating tumor progression of treated tumors in melanoma model mice treated with control, IL-12, or IL-12 plus CD3 half-BiTE IT-EP therapy.

FIG. 26A. (A) Graph illustrating tumor progression in breast cancer model mice treated with control, IL-12, or IL-12 plus 2C11 IT-EP therapy.

FIG. 26. B-C. (B) Graph illustrating ng metastasis nodules in 4T1 breast cancer model mice treated with control, IL 12-2A or IL12-2A plus 2C11 IT-EP therapy. (C) Graph illustrating the absolute number of effector T cells (CD127-CD62L-CD3+) per μL peripheral blood in 4T1 breast cancer model mice treated with control, IL12-2A or IL12-2A plus 2C11 IT-EP therapy.

FIG. 27. Graphs illustrating (A) hIL12p70 protein secretion, and (B) hCXCL9 protein secretion in HEK293 cells following transfection with hIL12-2A, hCXCL9, and hIL12˜hCXCL9 expression vectors. Protein detected by ELISA, n=5.

FIG. 28A. Volcano plots displaying p-values and log 2 fold change for the indicated genes. Differential gene expression was examined in mice treated with mCXCL9 alone (top panel) and mice treated with mCXCL9 in combination with IL12 (bottom panel). Horizontal lines indicate False Discovery Rate (FDR) thresholds.

FIG. 28B. Graph illustrating ‘Cytotoxic immune cells’ cell type scores. Each cell type's score (Log 2 scale) has been centered to have mean 0.

FIG. 29A. Graph illustrating IL12 p70 expression in 48 hr post electroporation in tumor lysates from mice bearing B16.F10 tumors after treatment with 10 μg or 100 μg of IL12-2A (TAVO) on days 1, 5, and 8, or 100 μg of IL12-CXCL9 or CD3 half-BiTE-IL12 (SPARK) on each of days 1, 5, and 8 (n=8 animals; DuoSet ELISA DY419).

FIG. 29B-C Graph illustrating primary (B) and secondary (C) tumor growth in mice bearing B16.F10 tumors after treatment with 10 μg or 100 μg of IL12-2A (TAVO) on days 1, 5, and 8, or 100 μg of IL12-CXCL9 or CD3 half-BiTE˜IL12 (SPARK) on each of days 1, 5, and 8. (From left to right for each of days 0 and 12: 10 μg IL12-2A, SPARK, 100 μg of IL12-2A).

FIG. 30. Graph illustrating: (A) anti-CTLA4 scFv transfection supernatant binding to recombinant mCTLA-4/Fc, and (B) detection of anti-CLTA-4 scFv on RENCA tumor lysates.

FIG. 31. Graphic illustration of treatment schedule. TAVO=nucleic acid expression IL-12 administered by IT-EP. P=pembrolizumab.

FIG. 32. Graphs illustrating Ki-67⁺ CD8⁺ T cells in PBMCs in responders and nonresponders before and after treatment with IL-12 and pembrolizumab.

FIG. 33. Graph illustrating intratumoral CXCR3 transcript levels in responders and nonresponders before and after treatment with IL-12 and pembrolizumab.

FIG. 34. Graphs illustrating CD8⁺ CXCR3⁺ T cells in PBMCs 24 hours after IT-EP with either 50 μg IL-12 (TAVO⁺ (TAVO(P2A)) or 50 μg control (empty) vector (EV).

FIG. 35. Graph illustrating number of migrating cells isolated from draining lymph nodes in mice treated with IT-EP IL-12 (TAVO⁺) empty vector (EV) in the presence of absence of anti-CXCR3 antibodies.

FIG. 36. Graphs illustrating primary and contralateral tumor regression in mice treated with IT-EP IL-12 (TAVO⁺) in the presence or absence of anti-CXCR3 antibodies.

FIG. 37. Graph illustrating survival in tumor model mice treated with IT-EP IL-12 (TAVO⁺) in the presence or absence of anti-CXCR3 antibodies.

FIG. 38. Graph illustrating IFN-γ in CD8⁺ T cells in mice treated with IT-EP with either 2 μg or 50 μg empty vector (EV) or IL-12 (TAVO⁺).

FIG. 39. Graphs illustrating transcriptome analyzes in tumor model mice treated with IT-EP empty vector (EV), IL-12 (TAVO⁺) or IL-12 plus CXCL9.

FIG. 40. FACS analyses of CD8 T cells in tumor model mice treated with IT-EP empty vector (EV), IL-12 (TAVO⁺) or IL-12 plus CXCL9.

FIG. 41. Graphs illustrating enhancement of primary and contralateral tumor regression in tumor model mice treated sequentially with IT-EP IL-12 (TAVO⁺) and IT-EP CXCL9 (left bar each pair=TAVO⁺+EV+EV; right bar each pair TAVO⁺+pCXCL9+pCXCL9).

FIG. 42. Graph illustrating survival in tumor model mice treated sequentially with IT-EP IL-12 (TAVO⁺) and IT-EP CXCL9.

FIG. 43. Graphs illustrating CXCR3⁺ expression of CD8⁺ T cells from tumor of mice treated with IT-EP empty vector (EV), IL-12 (TAVO⁺), or IL-12˜CXCL9.

FIG. 44. Graphs illustrating enhancement of primary and contralateral tumor repression in tumor model mice treated with IT-EP IL-12 (TAVO⁺), or IL-12˜CXCL9 (left bar each pair=TAVO⁺; right bar each pair TAVO⁺+CXC).

FIG. 45. Graph illustrating survival of tumor model mice treated with IT-EP empty vector (EV), IL-12 (TAVO⁺), or IL-12˜CXCL9.

FIG. 46. Graphs illustrating survival of tumor model mice treated with IT-EP empty vector (EV), with or without anti-PD-1 therapy, IT-EP IL-12 (TAVO⁺) with or without anti-PD-1 therapy, sequential IT-EP IL-12 plus IT-EP CXCL9, or sequential IT-EP IL-12 plus IT-EP CXCL9 with or without anti-PD-1 therapy. In each group, increase survival was observed in mice treating with anti-PD-1 therapy.

FIG. 47. Graph illustrating percentage of proliferating CD3⁺ T cells after 4 days of co-culture with B16-F10 cells, which were transfected with EV or anti-CD3 scFv plasmid, with or without 100 ng/mL mIL-12. Co-cultures were initiated with similar numbers of CD3⁺ T cells and B16-F10 cells. CD3⁺ T cells were cultured with plate bound anti-CD3 as positive control (n=3).

FIG. 48. Graphs illustrating flow cytometric analysis of intracellular (A) IFNγ and (B) Granzyme B expression in CD8⁺ and CD4⁺ T cells after 3 days of co-culture with B16-F10 cells, which were transfected in various conditions as described in the graph above (n=3).

FIG. 49. Graphs illustrating (A) tumor volume and (B) spontaneous metastatic lung modules in 4T1 tumors treated with IT-EP using 50 μg of empty vector (EV) or IL-12 (TAVO(P2A)) on Day 0 followed by subsequent IT-EP on days 3 and 5 with 50 μg of EV or CD3 half-BiTE: T cell populations were measured 6 days post IT-EP treatment; *p<0.05, **p<0.01, ***p<0.001, ****p<0.0001.

FIG. 49C-E. Graphs illustrating (C) CD3⁺CD8⁺ T cells, (D) CD8⁺ CXCR3⁺ T cells, and (E) CD45⁺ CD3⁺ T cells in 4T1 tumors treated with IT-EP using 50 μg of empty vector (EV) or IL-12 (TAVO(P2A)) on Day 0 followed by subsequent IT-EP on days 3 and 5 with 50 μg of EV or CD3 half-BiTE: T cell populations were measured 6 days post IT-EP treatment; *p<0.05, **p<0.01, ***p<0.001, ****p<0.0001.

FIG. 49F-G. Graphs illustrating (F) effector T cells and (G) effector memory T cells in 4T1 tumors treated with IT-EP using 50 μg of empty vector (EV) or IL-12 (TAVO(P2A)) on Day 0 followed by subsequent IT-EP on days 3 and 5 with 50 μg of EV or CD3 half-BiTE: T cell populations were measured 6 days post IT-EP treatment; *p<0.05, **p<0.01, ***p<0.001, ****p<0.0001.

FIG. 50A-B. Graphs illustrating (A) percentage of TILs (derived from a patient with melanoma actively progressing on anti-PD-1 therapy) proliferating after 3 days of co-culture with HEK293T cells transfected with empty vector or CD3 half-BiTE (αCD3) with or without IL-12 (tumor infiltrated T cells cultured with plate bound anti-CD3 antibody as positive control) (n=3); (B) Percentage of PD-1 expression on CD8⁺ TILs after 3 days of co-culture with HEK293T cells transfected as in (A); #=below detection, *p<0.05, **p<0.01, ***p<0.001, ****p<0.0001, statistical significance determined by one-way ANOVA.

FIG. 50C. Graph illustrating ELISA measuring IFNγ in the conditioned media of co-cultures of TILs and HEK293T cells transfected as in (A). #=below detection, *p<0.05, **p<0.01, ***p<0.001, ****p<0.0001, statistical significance determined by one-way ANOVA. (each set of five bars, in order: EV without pIL-12, EV with pIL-12, anti-CD3 half-BiTE without pIL1-2, anti-CD3 half-BiTE with pIL-12, plate bound anti-CD3 antibody)

### DETAILED DESCRIPTION

### 1. Definitions

A “nucleic acid” includes both RNA and DNA. RNA and DNA include, but are not limited to, cDNA, genomic DNA, plasmid DNA, condensed nucleic acid, nucleic acid formulated with a delivery vector, nucleic acid formulated with cationic lipids, nucleic acid formulated with peptides or cationic polymers, RNA, and mRNA. Nucleic acid also includes modified RNA or DNA.

An “expression cassette” refers to a nuclei acid (RNA or DNA) coding sequence or segment of RNA or DNA that codes for an expression product (e.g., peptide(s) (i.e., polypeptide(s) or protein(s)) or RNA). An expression cassette can be present in a plasmid. An expression cassette is capable of expressing one or more polypeptides in a cell, such a mammalian cell. The expression cassette may comprise one or more sequences necessary for expression of the encoded expression product. The expression cassette may comprise one or more of an enhancer, a promoter, a terminator, and a polyA signal operably linked to the DNA coding sequence.

The term “plasmid” refers to a nucleic acid that includes at least one sequence encoding a polypeptide (such as any of the described expression cassettes) that is capable of being expressed in a mammalian cell. A plasmid can be a closed circular DNA molecule. A variety of sequences can be incorporated into a plasmid to alter expression of the coding sequence are to facilitate replication of the plasmid in a cell. Sequences can be used that influence transcription, stability of a messenger RNA (mRNA), RNA processing, or efficiency of translation. Such sequences include, but are not limited to, 5′ untranslated region (5′ UTR), promoter, introns, and 3′ untranslated region (3′ UTR). Plasmids can be manufactured in large scale quantities and/or in high yield. Plasmids can further be manufacture using cGMP manufacturing. Plasmids can be transformed into bacteria, such as E. coli. The DNA plasmids are can be formulated to be safe and effective for injection into a mammalian subject.

“Protein,” “peptide,” or “polypeptide” includes a contiguous string of two or more amino acids. A “protein sequence,” “peptide sequence,” “polypeptide sequence,” or “amino acid sequence” refers to a series of two or more amino acids in a protein, peptide, or polypeptide.

The terms “express” and “expression” mean allowing or causing the information in a gene, RNA or DNA sequence to become manifest; for example, producing a protein by activating the cellular functions involved in transcription and translation of a corresponding gene. A DNA sequence is expressed in or by a cell to form an expression product such as an RNA (e.g., mRNA) or a protein. The expression product itself may also be said to be expressed by the cell.

“Operably linked” refers to the juxtaposition of two or more components (e.g., a promoter and another sequence element) such that both components function normally and allow the possibility that at least one of the components can mediate a function that is exerted upon at least one of the other components. For example, a promoter operably linked to a coding sequence will direct RNA polymerase mediated transcription of the coding sequence into RNA, including mRNA, which may then be spliced (if it contains introns) and, optionally, translated into a protein encoded by the coding sequence. A coding sequence can be operably linked to one or more transcriptional or translational control sequences. A terminator/polyA signal operably linked to a gene terminates transcription of the gene into RNA and directs addition of a polyA signal onto the RNA.

A “promoter” is a DNA regulatory region capable of binding an RNA polymerase in a cell (e.g., directly or through other promoter-bound proteins or substances) and initiating transcription of a coding sequence. A promoter may comprise one or more additional regions or elements that influence transcription initiation rate, including, but not limited to, enhancers. A promoter can be, but is not limited to, a constitutively active promoter, a conditional promoter, an inducible promoter, or a cell-type specific promoter. Examples of promoters can be found, for example, in WO 2013/176772. The promoter can be, but is not limited to, a CMV promoter, a Igκ promoter, a mPGK promoter, a SV40 promoter, a β-actin promoter, an α-actin promoter, a SRα promoter, a herpes thymidine kinase promoter, a herpes simplex virus (HSV) promoter, a mouse mammary tumor virus long terminal repeat (LTR) promoter, an adenovirus major late promoter (Ad MLP), a rous sarcoma virus (RSV) promoter, and an EF1α promoter. The CMV promoter can be, but is not limited to, a CMV immediate early promoter, a human CMV promoter, a mouse CNV promoter, and a simian CMV promoter.

A “translation modification element” enables translation of two or more genes from a single transcript, Translation modification elements include Internal Ribosome Entry Sites (IRES), which allow for initiation of translation from an internal region of an mRNA, and 2A peptides, derived from picornavirus, which cause the ribosome to skip the synthesis of a peptide bond at the C-terminus of the element. Incorporation of a translation modulating element results in co-expression of two or more poly peptide from a single polycistronic (multicistronic) mRNA. 2A modulators include, but are not limited to, P2A, T2A, E2A or F2A. 2A modulators contain a PG/P cleavage site.

A “homologous” sequence (e.g., nucleic acid sequence or amino acid sequence) refers to a sequence that is either identical or substantially similar to a known reference sequence, such that it is, for example, at least 75%, at least 80%, at least 85%, at least 90%, at least 95%, at least 96%, at least 97%, at least 98%, at least 99%, or 100% identical to the known reference sequence. “Orthologous” genes (Orthologs) include genes in different species that evolved from a common ancestral gene by speciation. Orthologs typically retain the same function in the course of evolution. Sequence identity can be determined by aligning sequences using algorithms, such as BESTFIT, FASTA, and TFASTA in the Wisconsin Genetics Software Package Release 7.0, Genetics Computer Group, 575 Science Dr., Madison, Wis.), using default gap parameters, or by inspection, and the best alignment (i.e., resulting in the highest percentage of sequence similarity over a comparison window). Percentage of sequence identity is calculated by comparing two optimally aligned sequences over a window of comparison, determining the number of positions at which the identical residues occurs in both sequences to yield the number of matched positions, dividing the number of matched positions by the total number of matched and mismatched positions not counting gaps in the window of comparison (i.e., the window size), and multiplying the result by 100 to yield the percentage of sequence identity. Unless otherwise indicated the window of comparison between two sequences is defined by the entire length of the shorter of the two sequences

“Immunostimulatory cytokine” includes cytokines that mediate or enhance the immune response to a foreign antigen, including viral, bacterial, or tumor antigens. Immunostimulatory cytokines can include, but are not limited to: TNFα, IL-1, IL-10, IL-12, IL-12 p35, IL-12 p40, IL-15, IL-15Rα, IL-23, IL-27, IFNα, IFNβ, IFNγ, IL-2, IL-4, IL-5, IL-7, IL-9, IL-21, and TGFβ.

“Cancer immunotherapy” is a therapy used to treat cancer that involves or uses components of the immune system. Cancer immunotherapy can induce, alter, or enhance a subject's immune system to fight cancer. Cancer immunotherapies include, but are not limited to, antibodies that bind to, inhibit, or alter the function of, proteins expressed by cancer cells or immune cells (targeted antibodies), cytokines, interferons, interleukins, and chemokines.

The term “cancer” includes a myriad of diseases generally characterized by inappropnate cellular proliferation, or abnormal or excessive cellular proliferation. Examples of cancer include, but are not limited to, breast cancer, triple negative breast cancer, colon cancer, prostate cancer, pancreatic cancer, melanoma, lung cancer, ovarian cancer, kidney cancer, brain cancer, or sarcomas.

A “treatment-refractory cancer” (or refractory cancer) is a cancer that does not respond, or has not responded, to at least one prior medical treatment. In some embodiments, a treatment-refractory cancer, with respect to a treatment, indicates an inadequate response to a treatment or the lack of a partial or complete response to the treatment. For example, patients may be considered refractory to a treatment, (e.g., checkpoint inhibitor therapy such as a PD-1 or PD-L1 inhibitor therapy) if they do not show at least a partial response after receiving at least 2 doses of the treatment. A refractory cancer can be resistant to a treatment before or at the beginning of the treatment. A refractory cancer can become refractory during the course of treatment.

A “responder” is a subject that has achieved, or is achieving, a complete response to an anti-cancer therapy. A “non-responder” is a subject that has not achieved, or is not achieving, an adequate response to an anticancer therapy. A non-responder may have a partial response, stable disease, progressive disease, increase in cancer cell number, or continued or increased tumor metastasis. Evaluation of subjects in assessing response, symptoms, and/or severity of the disease may be carried out by various methods, which are known in the art.

The “tumor microenvironment” refers to the environment around a tumor and includes the non-malignant vascular and stromal tissue that aid in growth and/or survival of a tumor, such as by providing the tumor with oxygen, growth factors, and nutrients, or inhibiting immune response to the tumor. A tumor microenvironment includes the cellular environment in which the tumor exists, including surrounding blood vessels, immune cells, fibroblasts, bone marrow-derived inflammatory cells, lymphocytes, signaling molecules and the extracellular matrix.

The “tumor margin” or “margin tissue” is the visually normal tissue immediately near or surrounding a tumor. Typically, the margin tissue is the visually normal tissue within 0.1-2 cm of the tissue. Tumor margin tissue is often removed when a tumor is surgically resected.

The term “treatment” includes, but is not limited to, a medicament or therapy for inhibition or reduction of proliferation of cancer cells, destruction of cancer cells, prevention of proliferation of cancer cells, prevention of initiation of malignant cells, arrest or reversal of the progression of transformed premalignant cells to malignant disease, or amelioration of the disease.

The term “electroporation” refers to the use of an electroporative pulse to facilitate entry of biomolecules such as a plasmid, nucleic acid, or drug, into a cell.

A “draining lymph node” is a lymph node that filters lymph from a particular region or organ. In context of tumors and tumor treatment, a draining lymph node lies immediately downstream of the tumor.

An “epitope tag” is a short amino acid sequence (or nucleic acid sequence encoding the short amino acid sequence) to which a high affinity antibody binds. Exemplary epitope tags include, but are not limited to, V5-tag, Myc-tag, HA-tag, Spot-tag, T7-tag, and NE-tag. Epitope tags can be used to facilitate immunodetection.

A tumor sample refers to a portion, piece, part, segment, or fraction of a tumor or tumor infiltrating lymphocytes from a subject. A tumor sample may be obtained from or removed from a subject using methods known in the art. Exemplary methods include, but are not limited to, surgical resection, biopsy, needle biopsy, or other means for obtaining a sample that contains a portion, piece, part, segment, or fraction of a tumor or tumor infiltrating lymphocytes. A tumor sample may be from any solid tumor, including primary tumors, invasive tumors, and metastatic tumors. The tumor sample may undergo additional purification and processing, for example, to remove cell debris and other unwanted molecules. Additional processing may further involve amplification, e.g., using PCR (RT-PCR). For measuring CXCR3 level or expression in a tumor sample, the tumor sample may be purified or processed using methods known in the art appropriate for a particular quantitation test or assay used in the analysis.

### II. CXCR3

Chemokine receptor CXCR3 is a Gαᵢ protein-coupled receptor in the CXC chemokine receptor family. Other names for CXCR3 are G protein-coupled receptor 9 (GPR9) and CD183. CXCR3 binds to the CXC chemokines CXCL9, CXCL10, and CXCL11. CXCR3 is expressed primarily on activated T lymphocytes and NK cells. CXCR3 is preferentially expressed on Th1 cells. CXCR3 is able to regulate leukocyte trafficking. CXCR3-ligand interaction attracts Th1 cells and promotes Th1 cell maturation. Expression of CXCR3 on leukocytes has been reported to mediate their migration to the tumor or tumor environment.

Described are methods of predicting response to checkpoint inhibitor therapy and/or immunostimulatory cytokine therapy comprising measuring CXCR3 level or expression in a tumor sample obtained from the subject. In some embodiments, CXCR3 level or expression in the tumor or tumor microenvironment is measured after administering at least one dose of a checkpoint inhibitor and/or an immunostimulatory cytokine to the subject. CXCR3 level or expression may be determined by measuring CXCR3 mRNA in the tumor sample, measuring CXCR3 protein in the tumor sample, or measuring CXCR3⁺ T cells in the tumor sample. In some embodiments, the at least one dose of a checkpoint inhibitor and/or an immunostimulatory cytokine comprises a dose that would be typically be considered pharmaceutically effective in responsive subjects.

CXCR3 level or expression in a tumor sample may be measured using tests or assays known in the art for measuring the amount or level of gene or protein expression. In some embodiments, the test or assay is an FDA-approved test or assay. In some embodiments, the level of CXCR3 expression in a tumor sample is determined by measuring the level of CXCR3 mRNA in the tumor sample. Exemplary methods of measuring the level of CXCR3 mRNA in a sample, include, but are not limited to, nucleic acid amplification assays, polymerase chain reaction (PCR) assays, real time PCR, TaqMan-based assays, hybridization assays, and microarray assays. In some embodiments, the level of CXCR3 expression in a tumor sample is determined by measuring the level of CXCR3 protein in the tumor sample. Exemplary methods of measuring the level of CXCR3 protein in a sample, include, but are not limited to, immune-based detection assays (immunoassays), such as Enzyme-Linked Immunosorbent Assays (ELISA) and AlphaLISA. In some embodiments, the level of CXCR3 expression in a tumor sample is determined by measuring the number of CXCR3⁺ T cells in the tumor sample. Exemplary methods of measuring the number of CXCR3⁺ T cells in a sample, include, but are not limited to, cell sorting assays.

In some embodiments, the tumor sample is obtained from the subject prior to anticancer therapy. In some embodiments, a tumor sample is obtained from a subject after at least one round of anticancer therapy. In some embodiments, a tumor sample is obtained from a subject after at least one round of checkpoint inhibitor therapy. In some embodiments, a tumor sample is obtained from a subject after at least one round immunostimulatory cytokine therapy. In some embodiments, a tumor sample is obtained from a subject after at least one round of checkpoint inhibitor plus immunostimulatory cytokine therapy. In some embodiments, the tumor sample is obtained from the subject 1-30 days after the at least one round of checkpoint inhibitor therapy and/or immunostimulatory cytokine therapy. In some embodiments, the tumor sample is obtained from the subject 1-21 days after the at least one round of checkpoint inhibitor therapy and/or immunostimulatory cytokine therapy. In some embodiments, the tumor sample is obtained from the subject 1, 2, 3, 4, 5, 6, 7, 8, 9, 10, 11, 12, 13, 14, 15, 16, 17, 18, 19, 20, or 21 days after the at least one round of checkpoint inhibitor therapy and/or immunostimulatory cytokine therapy. The checkpoint inhibitor therapy can be, but is not limited to anti-PD-1/anti-PD-L1 therapy. The anti-PD-1/anti-PD-L1 therapy may be administered systemically. The immunostimulatory cytokine can be, but is not limited to, IL-12 and/or IL-15 therapy. IL-12 and/or IL-15 therapy can be administered by intratumoral electroporation of a nucleic acid encoding IL-12 and/or IL-15.

CXCR3 expression as measured in a tumor sample obtained from a subject is compared with CXCR3 expression as measured in a predetermined control. The level of CXCR3 expression as determined in the tumor sample obtained from the subject is measured using the same or substantially the same method as used to measure the level of CXCR3 expression in the predetermined control.

In some embodiments, the tumor sample is obtained from a subject after administering at least one round of checkpoint inhibitor therapy or immunostimulatory cytokine therapy (treatment) to the subject and the predetermined control comprises a tumor sample obtained from the subject prior to the administering checkpoint inhibitor therapy or immunostimulatory cytokine therapy (treatment) to the subject. The level of CXCR3 expression as measured in the tumor sample from a subject obtained after administering treatment is compared with the level CXCR3 expression as measured in the predetermined control. In some embodiments, a level of CXCR3 expression as measured in the tumor sample obtained after treatment that is higher relative to the level CXCR3 expression as measured in the predetermined control indicates the subject is likely to respond to the checkpoint inhibitor therapy and/or immunostimulatory cytokine therapy, and a level of CXCR3 expression as measured in the tumor sample obtained after treatment that is the same or lower relative to the level CXCR3 expression as measured in the predetermined control indicates the subject is at risk of not responding to the checkpoint inhibitor therapy and/or immunostimulatory cytokine therapy. In some embodiments, a level of CXCR3 expression as measured in the tumor sample obtained after treatment that is more than twice the level CXCR3 expression as measured in the predetermined control indicates the subject is likely to respond to the checkpoint inhibitor therapy and/or immunostimulatory cytokine therapy, and a level of CXCR3 expression as measured in the tumor sample obtained after treatment that less than twice the level CXCR3 expression as measured in the predetermined control indicates the subject is at risk of not responding to the checkpoint inhibitor therapy and/or immunostimulatory cytokine therapy. In some embodiments, a level of CXCR3 expression as measured in the tumor sample obtained after treatment that is more than 1.9×, 1.8×, 1.7×, 1.6×, 1.5, 1.4×, 1.3×, 1.2×, or 1.1× the level CXCR3 expression as measured in the predetermined control indicates the subject is likely to respond to the checkpoint inhibitor therapy and/or immunostimulatory cytokine therapy. In some embodiments, a level of CXCR3 expression as measured in the tumor sample obtained after treatment that less than 1.9×, 1.8×, 1.7×, 1.6×, 1.5, 1.4×, 1.3×, 1.2×, or 1.1× the level CXCR3 expression as measured in the predetermined control indicates the subject is at risk of not responding to the checkpoint inhibitor therapy and/or immunostimulatory cytokine therapy.

In some embodiments, the predetermined control comprises a standard derived from a population of known responders and/or known non-responders to checkpoint inhibitor therapy. In some embodiments, the predetermined control comprises a standard derived from a population of known responders and/or known non-responders to immunostimulatory cytokine therapy. In some embodiments, the predetermined control comprises a standard derived from a population of known responders and/or known non-responders to checkpoint inhibitor plus immunostimulatory cytokine combination therapy. The level of CXCR3 expression as determined for the population of known responders and/or known non-responders is measured using the same or substantially the same method as used to measure the level of CXCR3 expression in the tumor sample obtained from the subject. A level of CXCR3 expression in a tumor sample obtained from a subject that is the same as or higher relative to a level of CXCR3 expression as determined for a population of known responders indicates the subject is likely to respond to checkpoint inhibitor and/or immunostimulatory cytokine therapy. A level of CXCR3 expression in a tumor sample obtained from a subject that is lower relative to a level of CXCR3 expression as determined for a population of known responders or is the same as or lower relative to a level of CXCR3 expression as determined for a population of known non-responders indicates the subject is likely at risk of not responding to checkpoint inhibitor and/or immunostimulatory cytokine therapy. In some embodiments, the tumor sample is obtained from the subject prior to administration of checkpoint inhibitor and/or immunostimulatory cytokine therapy. In some embodiments, the tumor sample is obtained from the subject after administration of at least one round of checkpoint inhibitor and/or immunostimulatory cytokine therapy. In some embodiments, the tumor sample is obtained from the subject concurrent with administration of at least one round of checkpoint inhibitor and/or immunostimulatory cytokine therapy. The level of CXCR3 expression in a population of known responders and/or known-non-responders may be calculated as or expressed as an average or mean of the levels of CXCR3 expression as measured in the known responders and/or known non-responders.

### III. CXCL9

C-X-C Motif Chemokine ligand 9 (CXCL9) is a small cytokine belonging to the CXC chemokine family. CXCL9 is also known as Monokine Induced by Gamma interferon (MIG). CXCL9 is a T-cell chemoattractant, and facilitates chemotactic recruitment of tumor infiltrating lymphocytes (TIL). The mouse and human CXCL9 amino acid sequences are represented by SEQ ID NO: 35 and SEQ ID NO: 58, respectively. In some embodiments, a CXCL9 comprises: (a) the amino acid sequence of SEQ ID NO: 35 or 58 or a functional equivalent thereof; or (b) an amino acid sequence having at least 90%, at least 95%, at least 96%, at least 97%, at least 98%, or at least 99% identify to the amino acid sequence of SEQ ID NO: 35 or 58.

IV. Anti-CTLA-4 scFv

An anti-CTLA-4 scFv comprises an anti-CTLA-4 single-chain variable fragment (scFv) having affinity for an extracellular domain of CTLA-4 and/or inhibiting CTLA-4 signaling. An scFv comprises a fusion protein of the variable regions of the heavy (VH) and light chains (VL) of immunoglobulins, connected with a short linker peptide. Exemplary mouse anti-CTLA-4 heavy chain variable region amino acid sequences are represented by SEQ ID NO: 39 and 43. Exemplary mouse anti-CTLA-4 light chain variable region amino acid sequences are represented by SEQ ID NO: 37 and 41.

An anti-CTLA-4 scFv can be identified from phage display. An anti-CTLA-4 scFv can also be generated by subcloning the VH and VL from a known anti-CTLA-4 antibody, such as from a hybridoma. Known anti-CTLA-4 antibodies have been described, for instance in 20190048096, 20130136749, 20120148597, 20140099325, 20150104409, 20110296546, and 20080233122, among others. Known anti-CTLA-4 antibodies include, but are not limited to, ipilimumab and tremelimumab. In some embodiments, the VH and or VL domains of an anti-CTLA-4 scFv can be humanized. A humanized antibody (or antibody fragment or domain) is an antibody from a non-human species whose protein sequences have been modified to increase their similarity to antibody variants produced naturally in humans. In some embodiments, humanized antibodies can be made by inserting the relevant complementarity-determining regions (CDRs, also termed hypervariable regions (HVRs)) of an anti-CTLA-4 antibody into human VH and VL domain scaffolds.

An anti-CTLA-4 scFv can be formed by linking the C-terminus of the VH chain with the N-terminus of the VL. Alternatively, the C-terminus of the VL can be linked to the N-terminus of the VH. The peptide linker can be about 10 to about 25 amino acids. In some embodiments, the scFv peptide linker is rich in glycine. An scFv peptide linker can be, but is not limited to, (G₄S)ₓ where x is an integer from 2 to 5 (inclusive). In some embodiments, the scFv peptide linked comprises Gly-Gly-Gly-Gly-Gly-Ser-Gly-Gly-Gly-Gly-Ser-Gly-Gly-Gly-Gly-Ser (i.e., also termed [(Gly)₄Ser]₃, (G₄S)₃ or G₄S (×3)). In some embodiments, the scFv peptide linker consists of G₄S (×3). In some embodiments, the encoded anti-CTLA-4 scFv polypeptide includes a signal peptide such as an Igκ signal peptide. Exemplary anti-CTLA-4 scFv amino acid sequences are represented by SEQ ID NO: 70 and 72. In some embodiments, an anti-CTLA-4 scFv comprises: (a) the amino acid sequence of SEQ ID NO: 70 or 72 or a functional equivalent thereof; or (b) an amino acid sequence having at least 90%, at least 95%, at least 96%, at least 97%, at least 98%, or at least 99% identify to the amino acid sequence of SEQ ID NO: 70 or 72.

### V. CD3 Half-BiTE

A CD3 half-BiTE comprises an anti-CD3 single-chain variable fragment (scFv) fused to a transmembrane domain (TM). An scFv comprises a fusion protein of the variable regions of the heavy (VH) and light chains (VL) of immunoglobulins, connected with a short linker peptide. Exemplary anti-CD3 heavy chain variable region amino acid sequences are represented by SEQ ID NO: 8 and 47. Exemplary mouse anti-CD3 light chain variable region amino acid sequences are represented by SEQ ID NO: II and 50.

An anti-CD3 scFv can be identified from phage display. An anti-CD3 scFv can also be generated by subcloning the VH and VL from a known anti-CD3 antibody, such as from a hybridoma. Known anti-CD3 antibodies have been described, for instance in US20180117152, US20140193399, US20100183554, and US20060177896. Known anti-CD3 antibodies also include, but are not limited to, OKT3 (Muromonab-CD3). 145-2C11, 17A2, SP7, and UCHTI. In some embodiments, the VH and or VL domains of an anti-CD3 scFv can be humanized. A humanized antibody (or antibody fragment or domain) is an antibody from a non-human species whose protein sequences have been modified to increase their similarity to antibody variants produced naturally in humans. In some embodiments, humanized antibodies can be made by inserting the relevant complementarity-determining regions (CDRs, also termed hypervariable regions (HVRs)) of an anti-CD3 antibody into human VH and VL domain scaffolds.

An anti-CD3 scFv can be formed by linking the C-terminus of the VH chain with the N-terminus of the VL. Alternatively, the C-terminus of the VL can be linked to the N-terminus of the VH. The peptide linker can be about 10 to about 25 amino acids. In some embodiments, the scFv peptide linker is rich in glycine. An scFv peptide linker can be, but is not limited to, (G₄S)ₓ where x is an integer from 2 to 5 (inclusive). In some embodiments, the scFv peptide linker comprises Gly-Gly-Gly-Gly-Gly-Ser-Gly-Gly-Gly-Gly-Ser-Gly-Gly-Gly-Gly-Ser (i.e., also termed [(Gly)₄Ser]₃, (G₄S)₃ or G₄S (×3)). In some embodiments, the scFv peptide linker consists of G₄S (×3).

A transmembrane domain (TM) comprises a polypeptide capable of being inserted into a biological lipid bilayer (membrane) and anchoring the CD3 half-BiTE to the membrane. TMs are known in the art and typically consist predominantly of nonpolar amino acids. The transmembrane domain can be, but is not limited to, a PDGFRβ transmembrane domain or a PDGFRα transmembrane domain (PDGFR is Platelet-derived growth factor receptor). In some embodiments, a spacer is included between the anti-CD3 scFv and the transmembrane domain. In some embodiments, the TM domain comprises an amino acid sequence selected from the group comprising: VCQDTQEVIVVPHSLPFKVVVISAILALVVLTIISLIILIMLWQKKPR (SEQ ID NO: 25), AVGQDTQEVIVVPHSLPFKVVVISAILALVVLTIISLIILIMLWQ KKPR (SEQ ID NO: 27), PDGFRβ: VVISAILALVVLTVISLIILI (SEQ ID NO: 83), PDGFRβ: VVISAILALVVLTIISLIILI (SEQ ID NO: 84), PDGFRα: AAVLVLLVIVIISLIVL VVIW (SEQ ID NO: 85), and PDGFRα: AAVLVLLVIVIVSLIVLVVIW (SEQ ID NO: 86). In some embodiments, the TM domain is encoded by a nucleic acid sequence selected from the group comprising: gtgggccaggacacgcaggaggtcatcgtggtgccacactccttgccctitaaggtggiggtgatctcag ccatcctggccctggtggtgctcaccatcatctcccttatcatcctcatcatgctttggcagaagaagccacgt (SEQ ID NO: 24), gctgtgggccaggacacgcaggaggtcatcgtggtgccacactccttgccctttaaggtggtggtgatctcagccatcctggcc ctggtggtgctcaccatcatctcccttatcatcctcatcatgctttggcagaagaagccacgt (SEQ ID NO: 26), PDGFRβ: tggtgatctcagccatcctggccctggtggtgctcaccatcatetcccttatcatcctcatc (SEQ ID NO: 87), PDGFRβ: gtggtgatctcagccatcctggccctggtggtgctcaccatcatctcccttatcatcctcatc (SEQ ID NO: 88), PDGFRα: gctgcagtcctggtgctgttggtgattgtgatcatctcacttattgtcctggttgicatttgga (SEQ ID NO: 89).

In some embodiments, the encoded CD3 half-BiTE polypeptide includes a signal peptide such as an Igκ signal peptide.

Exemplary CD3 half-BiTE amino acid sequences are represented by SEQ ID NO: 60, 62, 74, and 76. In some embodiments, a CD3 half-BiTE comprises: (a) the amino acid sequence of SEQ ID NO: 60, 62, 74, or 76 or a functional equivalent thereof; or (b) an amino acid sequence having at least 90%, at least 95%, at least 96%, at least 97%, at least 98%, or at least 99% identify to the amino acid sequence of SEQ ID NO: 60, 62, 74, or 76.

### VI. Expression Cassettes

Any of the described polypeptides, CXCL9, CD3 half-BiTE, anti-CTLA4 scFv, and IL-12, may be encoded on a nucleic acid. The nucleic acid can be, but is not limited to, an expression cassette. The expression cassette can be on a plasmid. The term “plasmid” includes any nucleic acid vector including a bacterial vector, a viral vector, an episomal plasmid, an integrative plasmid, or a phage vector. Delivery of an expression cassette includes delivery of a plasmid or nucleic acid vector (as termed “expression vector” or “vector”) containing the expression cassette.

An encoded polypeptide may be linked, in an expression cassette, to a sequence encoding a second polypeptide. In some embodiments, an expression cassette encodes a fusion protein. The term “fusion protein” refers to a protein comprising two or more polypeptides linked together by peptide bonds or other chemical bonds. In some embodiments, a fusion protein is recombinantly expressed as a single-chain polypeptide containing the two polypeptides. The two or more polypeptides can be linked directly or via a linker comprising one or more amino acids.

An expression cassette or plasmid may contain a multicistronic expression cassette. Multicistronic expression cassettes express two or more separate proteins from the same mRNA and contain one or more translation modification elements.

In some embodiments, the described expression cassettes encode two or three polypeptides expressed from a single promoter, with one or more translation modification elements to allow the two or three polypeptides to be expressed from a single mRNA. In some embodiments, the expression cassettes comprise:

| | |
|---|---|
| a) | P-A-T-B, |
| b) | P-B-T-A, |
| c) | P-B-T-B′ |
| c) | P-A-T-B-T′-B′ or |
| d) | P-B-T-B′-T′-A |

wherein P is a promoter, A encodes CXCL9 or a CD3 half-BiTE, B and B′ encode cytokines or cytokine subunits, and T and T′ are translation modification elements.

A promoter can be, but is not limited to, a constitutively active promoter, a conditional promoter, an inducible promoter, or a cell-type specific promoter. Examples of promoters can be found, for example, in WO 2013/176772 The promoter can be, but is not limited to, a CMV promoter, a Igκ promoter, a mPGK promoter, a SV40 promoter, a β-actin promoter, an α-actin promoter, a SRα promoter, a herpes thymidine kinase promoter, a herpes simplex virus (HSV) promoter, a mouse mammary tumor virus long terminal repeat (LTR) promoter, an adenovirus major late promoter (Ad MLP), a rous sarcoma virus (RSV) promoter, and an EF1α promoter. A CMV promoter can be, but is not limited to, a CMV immediate early promoter, a human CMV promoter, a mouse CNV promoter, and a simian CMV promoter.

In some embodiments, T and/or T′ are internal ribosome entry site (IRES) elements or ribosomal skipping modulators. A ribosome skipping modulator can be, but is not limited to, a 2A element (also termed 2A peptide or 2A self-cleaving peptide). The 2A element can be, but is not limited to, a P2A (SEQ ID NO: 29), T2A, E2A or F2A element.

The CXCL9 can be, but is not limited to, mouse CXCL9 and human CXCL9, or a functional equivalent or homolog or ortholog thereof.

The CD3 half-BiTE can be, but is not limited to: ant-CD3 scFv-transmembrane domain (TM), epitope tag (ET)-anti-CD3 scFv-ET-TM, ET-anti-CD3 scFv-TM, anti-CD3, scFv-ET-TM, HA-anti-CD3 scFv-Myc-TM, HA-anti-CD3 scFv-TM, anti-CD3, scFv-Myc-TM, anti-CD3 scFv-TM, or anti-CD3 scFv-TM. The anti-CD3 scFv can be an anti-mouse CD3 scFv or an anti-human CD3 scFv. Each of these can include a signal peptide. The signal peptide can be, but is not limited to, an Igκ signal peptide. The TM can be, but is not limited to, a PDGFR TM. The anti-CD3 scFv can be, but is not limited to, 2C11 or OKT3.

In some embodiments, the cytokine is an immunostimulatory cytokine. In some embodiments, the immunostimulatory cytokine is an interleukin. Cytokines include, but are not limited to, IL-1, IL-2, IL-10, IL-12, IL-15, IL-23, IL-27, IL-35, IFN-α, IFN-β, IFN-γ, and TGF-β. In some embodiments, B and/or B′ encode an IL-12, IL-12 p35-TL-12 p40 fusion, TL-12 p70, IL-12 p35, or IL-12 p40 polypeptide. The TL-12, IL-12 p35-IL-12 p40 fusion, IL-12 p70, IL-12 p35, or IL-12 p40 polypeptide may be, but is not limited to, a mouse or human IL-12, IL-12 p35-IL-12 p40 fusion, IL-12 p70. IL-12 p35, or IL-12 p40 polypeptide. In some embodiments, B encodes IL-12 p35 and B′ encodes IL-12 p40.

In some embodiments P is a CMV promoter, A encodes CXCL9, T is a P2A element, B encodes IL-12 p35 and B′ encodes IL-12 p40.

In some embodiments P is a CMV promoter, A encodes a human CXCL9, T is a P2A element. B encodes IL-12 p35 and B′ encodes IL-12 p40.

In some embodiments P is a CMV promoter, A encodes a mouse CXCL9, T is a P2A element, B encodes IL-12 p35 and B′ encodes IL-12 p40.

In some embodiments P is a CMV promoter, A encodes an Igκ-HA-anti-CD3 scFv-PDGFR TM CD3 half-BiTE, T is a P2A element, B encodes IL-12 p35 and B′ encodes IL-12 p40.

In some embodiments P is a CMV promoter, A encodes an Ig-anti-CD3 scFv-PDGFR TM CD3 half-BiTE, T is a P2A element, B encodes IL-12 p35 and B′ encodes IL-12 p40.

In some embodiments P is a CMV promoter, A encodes an Igκ-HA-2C11-PDGFR TM CD3 half-BiTE, T is a P2A element, B encodes IL-12 p35 and B′ encodes IL-12 p40.

In some embodiments P is a CMV promoter, A encodes an Igκ-2C11-PDGFR TM CD3 half-BiTE, T is a P2A element, B encodes IL-12 p35 and B′ encodes IL-12 p40.

In some embodiments P is a CMV promoter, A encodes an Igκ-HA-OCT3-PDGFR TM CD3 half-BiTE, T is a P2A element, B encodes IL-12 p35 and B′ encodes IL-12 p40.

In some embodiments P is a CMV promoter, A encodes an Igκ-OKT3-PDGFR TM CD3 half-BiTE, T is a P2A element, B encodes TL-12 p35 and B′ encodes IL-12 p40.

In some embodiments, B encodes IL-12 p35, T is a P2A element, and B′ encodes IL-12 p40. In some embodiments, B encodes IL-12 p35, T is an IRES element, and B′ encodes IL-12 p40. The promoter can be, but is not limited to, a CMV promoter.

In some embodiments, we describe expression cassettes encoding a polypeptide comprising the amino acid sequence of SEQ ID NO: 60, 62, 74, or 76 or a polypeptide having at least 70% identity to the amino acid sequence of SEQ ID NO: 60, 62, 74, or 76. In some embodiments, an expression cassette encodes a polypeptide comprising an amino acid sequence having greater than 70%, 72%, 75%, 78%, 800, 82%, 83%, 85%, 87%, 88%, 90%, 92%, 93%, 95%, 96%, 97%, 98%, or 99% identity to the amino acid sequence of SEQ ID NO: 60, 62, 74, or 76, wherein the encoded polypeptide retains the functional activity of an CD3 half-BiTE polypeptide.

In some embodiments, we describe expression cassettes encoding a polypeptide comprising the amino acid sequence of SEQ ID NO: 64, 66, 78, or 70, or a polypeptide having at least 70% identity to the amino acid sequence of SEQ ID NO: 64, 66, 78, or 70. In some embodiments, an expression cassette encodes a polypeptide comprising an amino acid sequence having greater than 70%, 72%, 75%, 78%, 80%, 82%, 83%, 85%, 87%, 88%, 90%, 92%, 93%, 95%, 96%, 97%, 98%, or 99% identity to the amino acid sequence of SEQ ID NO: 64, 66, 78, or 70, wherein encoded the polypeptides retain the functional activity of an CD3 half-BiTE polypeptide and an IL-12 polypeptide.

In some embodiments, we describe expression cassettes encoding a polypeptide comprising the amino acid sequence of SEQ ID NO: 35 or 58, or a polypeptide having at least 70% identity to the amino acid sequence of SEQ ID NO: 35 or 58. In some embodiments, an expression cassette encodes a polypeptide comprising an amino acid sequence having greater than 70%, 72%, 75%, 78%, 80%, 82%, 83%, 85%, 87%, 88%, 90%, 92%, 93%, 95%, 96%, 97%, 98%, or 99% identity to the amino acid sequence of SEQ ID NO: 35 or 58, wherein the encoded polypeptide retains the functional activity of a CXCL9 polypeptide.

In some embodiments, we describe expression cassettes encoding a polypeptide comprising the amino acid sequence of SEQ ID NO: 68 or 82, or a polypeptide having at least 70% identity to the amino acid sequence of SEQ ID NO: 68 or 82. In some embodiments, an expression cassette encodes a polypeptide comprising an amino acid sequence having greater than 70%, 72%, 75%, 78%, 80%, 82%, 83%, 85%, 87% 88% 90% 92%, 93%, 95%, 96%, 97%, 98%, or 99% identity to the amino acid sequence of SEQ ID NO: 68 or 82, wherein encoded the polypeptides retain the functional activity of a CXCL9 polypeptide and an IL-12 polypeptide.

In some embodiments, we describe expression cassettes encoding a polypeptide comprising the amino acid sequence of SEQ ID NO: 70 or 72 or a polypeptide having at least 70% identity to the amino acid sequence of SEQ ID NO: 70 or 72. In some embodiments, an expression cassette encodes a polypeptide comprising an amino acid sequence having greater than 70%, 72%, 75%, 78%, 80%, 82%, 83%, 85%, 87%, 88%, 90%, 92%, 93%, 95%, 96%, 97%, 98%, or 99% identity to the amino acid sequence of SEQ ID NO: 70 or 72, wherein the encoded polypeptide retains the functional activity of an anti-CTLA-4 scFv polypeptide.

In some embodiments, we describe expression cassettes comprising the nucleotide sequence of SEQ ID NO: 59, 61, 73, or 75, or a nucleotide sequence having at least 70% identity to the nucleotide sequence of SEQ ID NO: 59, 61, 73, or 75. In some embodiments, an expression cassette comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 59, 61, 73, or 75 and encodes a polypeptide having the functional activity of an CD3 half-BiTE polypeptide. In some embodiments, the nucleotide sequence of SEQ ID NO: 59, 61, 73, or 75 or the nucleotide sequence having at least 70% identity to the nucleotide sequence of SEQ ID NO: 59, 61, 73, or 75 is operably linked to a CMV promoter.

In some embodiments, we describe expression cassettes comprising the nucleotide sequence of SEQ ID NO: 63, 65, 77, or 79, or a nucleotide sequence having at least 70% identity to the nucleotide sequence of SEQ ID NO: 63, 65, 77, or 79. In some embodiments, an expression cassette comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 63, 65, 77, or 79, and encode polypeptides having the functional activity of an CD3 half-BiTE polypeptide and an IL-12 polypeptide. In some embodiments, the nucleotide sequence of SEQ ID NO: 63, 65, 77, or 79 or the nucleotide sequence having at least 70% identity to the nucleotide sequence of SEQ ID NO: 63, 65, 77, or 79 is operably linked to a CMV promoter.

In some embodiments, we describe expression cassettes comprising the nucleotide sequence of SEQ ID NO: 34 or 57, or a nucleotide sequence having at least 70% identity to the nucleotide sequence of SEQ ID NO: 34 or 57. In some embodiments, an expression cassette comprises a sequence having greater than 70%, 72% 75%, 78%, 80%, 82%, 83%, 85%, 87%, 88%, 90%, 92%, 93%, 95%, 96%, 97%, 98%, or 99% identity to the nucleotide sequence of SEQ ID NO: 34 or 57, and encodes a polypeptide having the functional activity of a CXCL9 polypeptide. In some embodiments, the nucleotide sequence of SEQ ID NO: 34 or 57 or the nucleotide sequence having at least 70% identity to the nucleotide sequence of SEQ ID NO: 34 or 57 is operably linked to a CMV promoter.

In some embodiments, we describe expression cassettes comprising the nucleotide sequence of SEQ ID NO: 67 or 81 or a nucleotide sequence having at least 70% identity to the nucleotide sequence of SEQ ID NO: 67 or 81. In some embodiments, an expression cassette comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 67 or 81, and encodes polypeptides having the functional activity of a CXCL9 polypeptide and an IL-12 poly peptide. In some embodiments, the nucleotide sequence of SEQ ID NO: 67 or 81 or the nucleotide sequence having at least 70% identity to the nucleotide sequence of SEQ ID NO: 67 or 81 is operably linked to a CMV promoter.

In some embodiments, we describe expression cassettes comprising the nucleotide sequence of SEQ ID NO: 69 or 71, or a nucleotide sequence having at least 70% identity to the nucleotide sequence of SEQ ID NO: 69 or 71. In some embodiments, an expression cassette comprises a sequence having greater than 70%, 72%, 75%, 78%, 80%, 82%, 83%, 85%, 87%, 88%, 90%, 92%, 93%, 95%, 96%, 97%, 98%, or 99% identity to the nucleotide sequence of SEQ ID NO: 69 or 71, and encodes a polypeptide having the functional activity of an anti-CTLA-4 scFv polypeptide. In some embodiments, the nucleotide sequence of SEQ ID NO: 69 or 71 or the nucleotide sequence having at least 70% identity to the nucleotide sequence of SEQ ID NO: 69 or 71 is operably linked to a CMV promoter.

### VII. Methods of Treatment

Described are methods for treatment of a tumor in a subject comprising, administering a composition comprising an effective dose of one or more of the described CXCL9, CD3 half-BiTE, and or CTLA-4 scFv expression cassettes to the tumor, tumor microenvironment, and/or a tumor margin tissue and administering electroporation therapy to the tumor, tumor microenvironment, and/or a tumor margin tissue (IT-EP therapy). The CXCL9 or CD3 half-BiTE expression cassette may further encode IL-12. In some embodiments, the effective dose of the expression cassette is administered to the tumor, such as by injecting the expression cassette into the tumor and administering at least one electroporation pulse to the tumor.

The treated tumor can be a cutaneous tumor, a subcutaneous tumor, or a visceral tumor. The tumor can be cancerous or non-cancerous. The tumor can be, but is not limited to, a solid tumor, a surface lesion, a non-surface lesion, a lesion within 15 cm of body surface, or a visceral lesion. In some embodiments, the described methods and expression vectors can be used to treat primary tumors as well as distant (i.e., untreated) tumors and metastases. In some embodiments, the described methods provide for reducing the size of or inhibiting the growth of a tumor, inhibiting the growth of cancer cells, inhibiting or reducing metastasis, reducing or inhibiting the development of metastatic cancer, and/or reducing recurrence of cancer in a subject suffering from cancer. The tumor is not limited to a specific type of tumor or cancer.

In some embodiments, the methods further comprise administering an effective dose of an immunostimulatory cytokine. The immunostimulatory cytokine can be administered by IT-EP of an expression cassette encoding the cytokine. In some embodiments, the cytokine is encoded on the expression cassette encoding the CXCL9 or CD3 half-BiTE. In some embodiments, the cytokine is encoded on a second expression vector and delivered to the cancerous tumor by IT-EP. In some embodiments, the cytokine is IL-12. In some embodiments, the expression cassette comprises B-T-B′, wherein B encodes IL-12 p35, T is a P2A element, and B′ encodes IL-12 p40. The cytokine may be administered prior to, concurrent with, or subsequent to IT-EP CXCL9 therapy or IT-EP CD3 half-BiTE therapy.

IT-EP CXCL9 therapy or treatment comprises injecting a tumor, tumor microenvironment, and/or tumor margin tissue with an effective dose of a described expression cassette encoding CXCL9 and administering electroporation therapy to the tumor. In some embodiments, the expression cassette is injected into the tumor.

IT-EP IL12-CXCL9 therapy or treatment comprises injecting a tumor, tumor microenvironment, and/or tumor margin tissue with an effective dose of a described expression cassette encoding CXCL9 and IL-12 and administering electroporation therapy to the tumor. In some embodiments, the expression cassette is injected into the tumor.

IT-EP CD3 half-BiTE therapy or treatment comprises injecting a tumor, tumor microenvironment, and/or tumor margin tissue with an effective dose of a described expression cassette encoding a CD3 half-BiTE and administering electroporation therapy to the tumor. In some embodiments, the expression cassette is injected into the tumor.

IT-EP CD3 half-BiTE˜IL-12 or treatment therapy comprises injecting a tumor, tumor microenvironment, and/or tumor margin tissue with an effective dose of a described expression cassette encoding CD3 half-BiTE and IL-12 and administering electroporation therapy to the tumor. In some embodiments, the expression cassette is injected into the tumor.

IT-EP anti-CTLA-4 scFv therapy or treatment comprises injecting a tumor, tumor microenvironment, and/or tumor margin tissue with an effective dose of a described expression cassette encoding anti-CTLA-4 scFv and administering electroporation therapy to the tumor. In some embodiments, the expression cassette is injected into the tumor.

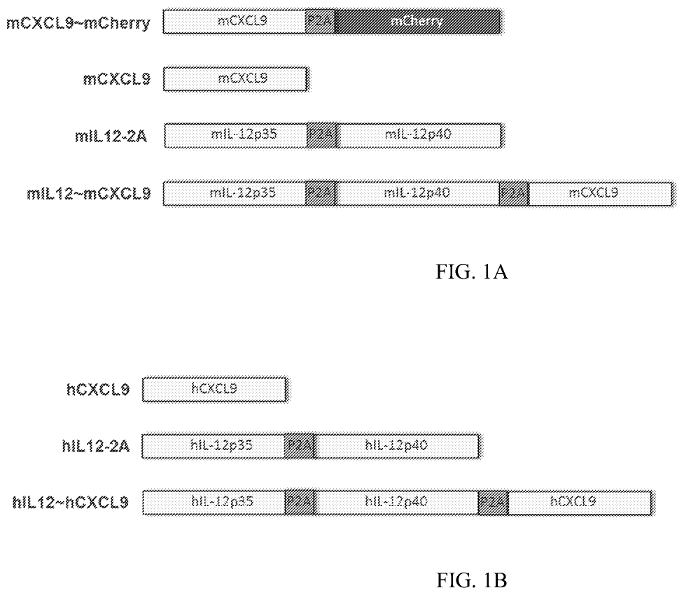

IT-EP IL12 therapy or treatment comprises injecting a tumor, tumor microenvironment, and/or tumor margin tissue with an effective dose of an expression cassette encoding IL-12 and administering electroporation therapy to the tumor. In some embodiments the expression cassette encoding IL-12 comprises IL12-2A (mIL12-2A and hIL12-2A; FIG. 1). In some embodiments, the expression cassette is injected into the tumor.

In some embodiments, the described expression cassettes, plasmids containing the described expression cassettes, and methods can be used to treat one or more tumors, tumor cells, or tumor lesions. The tumor cells can be, but are not limited to cancer cells. The term “cancer” includes a myriad of diseases generally characterized by inappropriate cellular proliferation, abnormal or excessive cellular proliferation. The cancer can be, but is not limited to, solid cancer, sarcoma, carcinoma, and lymphoma. The cancer can also be, but is not limited to, pancreas, skin, brain, liver, gall bladder, stomach, lymph node, breast, lung, head and neck, larynx, pharynx, lip, throat, heart, kidney, muscle, colon, prostate, thymus, testis, uterine, ovary, cutaneous, and subcutaneous cancers. Skin cancer can be, but is not limited to, melanoma and basal cell carcinoma. Breast cancer can be, but is not limited to, ER positive breast cancer, ER negative breast cancer, and triple negative breast cancer. In some embodiments, the described methods can be used to treat cell proliferative disorders. The term “cell proliferative disorder” denotes malignant as well as non-malignant cell populations which often appear to differ from the surrounding tissue both morphologically and genotypically. In some embodiments, the described methods can be used to treat a human. In some embodiments, the described methods can be used to treat non-human animals or mammals. A non-human mammal can be, but is not limited to, mouse, rat, rabbit, dog, cat, pig, cow, sheep, or horse.

The described expression cassettes and methods are contemplated for use in subjects afflicted with cancer or other non-cancerous (benign) growths. Tumors treated with the methods of the present embodiment may be any of noninvasive, invasive, superficial, papillary, flat, metastatic, localized, unicentric, multicentric, low grade, and high grade tumors. These growths may manifest themselves as any of a lesion, polyp, neoplasm (e.g. papillary urothelial neoplasm), papilloma, malignancy, tumor (e.g., Klatskin tumor, hilar tumor, noninvasive papillary urothelial tumor, germ cell tumor, Ewing's tumor, Askin's tumor, primitive neuroectodermal tumor, Leydig cell tumor, Wilms' tumor, Sertoli cell tumor), sarcoma, carcinoma (e.g. squamous cell carcinoma, cloacogenic carcinoma, adenocarcinoma, adenosquamous carcinoma, cholangiocarcinoma, hepatocellular carcinoma, invasive papillary urothelial carcinoma, flat urothelial carcinoma), lump, or any other type of cancerous or non-cancerous growth. The expression cassettes and methods can be used to treat advanced, metastatic, or treatment refractory cancer.

The expression cassettes and methods described herein are contemplated for use in, e.g., adrenal cortical cancer, anal cancer, bile duct cancer (e.g., periphilar cancer, distal bile duct cancer, intrahepatic bile duct cancer) bladder cancer, benign and cancerous bone cancer (e.g., osteoma, osteoid osteoma, osteoblastoma, osteochrondroma, hemangioma, chondromyxoid fibroma, osteosarcoma, chondrosarcoma, fibrosarcoma, malignant fibrous histiocytoma, giant cell tumor of the bone, chordoma, lymphoma, multiple myeloma), brain and central nervous system cancer (e.g., meningioma, astrocytoma, oligodendrogliomas, ependymoma, gliomas, medulloblastoma, ganglioglioma, Schwannoma, germinoma, craniopharyngioma), breast cancer (e.g., ductal carcinoma in situ, infiltrating ductal carcinoma, infiltrating lobular carcinoma, lobular carcinoma in situ, gynecomastia), Castleman disease (e.g., giant lymph node hyperplasia, angiofollicular lymph node hyperplasia), cervical cancer, colorectal cancer, endometrial cancer (e.g., endometrial adenocarcinoma, adenocanthoma, papillary serous adnocarcinoma, clear cell) esophagus cancer, gallbladder cancer (mucinous adenocarcinoma, small cell carcinoma), gastrointestinal carcinoid tumors (e.g., choriocarcinoma, chorioadenoma destruens), Hodgkin's disease, non-Hodgkin's lymphoma, Kaposi's sarcoma, kidney cancer (e.g., renal cell cancer), laryngeal and hypopharyngeal cancer, liver cancer (e.g., hemangioma, hepatic adenoma, focal nodular hyperplasia, hepatocellular carcinoma), lung cancer (e.g., small cell lung cancer, non-small cell lung cancer), mesothelioma, plasmacytoma, nasal cavity and paranasal sinus cancer (e.g., esthesioneuroblastoma, midline granuloma), nasopharyngeal cancer, neuroblastoma, oral cavity and oropharyngeal cancer, ovarian cancer, pancreatic cancer, penile cancer, pituitary cancer, prostate cancer, retinoblastoma, rhabdomyosarcoma (e.g., embryonal rhabdomyosarcoma, alveolar rhabdomyosarcoma, pleomorphic rhabdomyosarcoma), salivary gland cancer, skin cancer, both melanoma and non-melanoma skin cancer), stomach cancer, testicular cancer (e.g., seminoma, nonseminoma germ cell cancer), thymus cancer, thyroid cancer (e.g., follicular carcinoma, anaplastic carcinoma, poorly differentiated carcinoma, medullary thyroid carcinoma, thyroid lymphoma), vaginal cancer, vulvar cancer, and uterine cancer (e.g., uterine leiomyosarcoma).

In some embodiments, the subject has low tumor infiltrating lymphocytes (TILs) and/or impaired tumoral IFNγ signaling.

The described methods can be used to cause one or more of the following: inflame a tumor, induce T cell infiltration to the tumor or tumor microenvironment (increase the number of tumor infiltrating lymphocytes (TILs)), enhance systemic T cell response, induce activation of tumor-specific T cells, increase antigen-specific T cell response, increase proliferation of antigen-specific T cells, increase polyclonal T cells response, enhance an immune response against treated and/or untreated tumors, decrease T cell exhaustion, increase lymphocyte and monocyte cell surface markers in one or more treated or untreated tumors, increase intratumoral levels of INFγ regulated genes in one or more treated or untreated tumors, increase proliferating effector memory T cells in the subject's blood, increase short-lived effector cells in the subject's blood, increase expression of genes present in activated natural killer cells in a cancerous tumor, increase expression of genes that function in antigen presentation in a cancerous tumor, increase expression of genes that function in T cell survival and T cell mediated cytotoxicity in a cancerous tumor, induce regression of treated and/or untreated tumors, induce debulking of a treated and/or untreated tumor, and improve response to a second therapy, such as, but not limited to, immune checkpoint inhibitor therapy. In some embodiments, enhancement of immune reaction to the tumor leads to increased survival of the subject.

In some embodiments, the described methods of treating a subject having a cancerous tumor comprise: injecting the cancerous tumor with an effective dose of a plasmid encoding CXCL9, and administering electroporation therapy to the tumor. In some embodiments, the described methods of treating a subject having a cancerous tumor comprise: injecting the cancerous tumor with an effective dose of a plasmid encoding CD3 half-BiTE, and administering electroporation therapy to the tumor. In some embodiments, the described methods of treating a subject having a cancerous tumor comprise: injecting the cancerous tumor with an effective dose of a plasmid encoding an anti-CTLA-4 scFv, and administering electroporation therapy to the tumor. In some embodiments, the plasmid is administered substantially contemporaneously with the electroporation treatment. The term “substantially contemporaneously” means that the molecule and the electroporation treatment are administered reasonably close together with respect to time, i.e., before the effect of the electrical pulses on the cells diminishes.

In some embodiments, the described methods result in increased NK cells and T cell populations in a tumor or tumor microenvironment. IT-EP of CXCL9, IL12˜CXCL9, CD3 half-BiTE˜IL12, and/or CD3 half-BiTE increases homing of tumor-specific T cells to tumors, increases activation and/or proliferation of tumor-specific T cells, and/or increases recruitment of CD8⁺ T cells, NK cells, and NKT cells to the tumor microenvironment. Activation of T cells can lead to increased tumor cell killing by the activated T cells.

In some embodiments, administration of IL-12 therapy by IT-EP enhances T cell infiltration of the tumor. Subsequent expression of CD3 half-BiTE in the tumor can activate the T cells to enhance the population of antigen specific T cells.

In some embodiments, IT-EP CXCL9 therapy enhances an IL-12 effect resulting in increased effective trafficking of tumor specific lymphocytes.

In some embodiments, IT-EP CXCL9 therapy inhibits angiogenesis in a tumor or tumor microenvironment. In some embodiments, combining IT-EP CXCL9 with IL-12 therapy increases trafficking of tumor-specific lymphocytes to tumors.

In some embodiments, intratumoral electroporation of an expression cassette encoding a CXCL9 can be administered with other therapeutic entities. In some embodiments, IT-EP CXCL9 therapy is combined IL-12 therapy. IL-12 therapy may occur before, concurrent with, and/or after IT-EP CXCL9 therapy. IL-12 therapy can occur before and concurrent with IT-EP CXCL9 therapy. IL-12 therapy can occur before and after IT-EP CXCL9 therapy. IL-12 therapy can occur concurrent with and after IT-EP CXCL9 therapy. IL-12 therapy may occur before, concurrent with, and after IT-EP CXCL9 therapy. IT-EP CXCL9 therapy may occur before, concurrent with, and/or after IL-12 therapy. IT-EP CXCL9 therapy may occur before and concurrent with IL-12 therapy. IT-EP CXCL9 therapy may occur before and after IL-12 therapy. IT-EP CXCL9 therapy may occur concurrent with and after IL-12 therapy. IT-EP CXCL9 therapy may occur before, concurrent with, and after IL-12 therapy. In some embodiments, the IL-12 therapy is administered by IT-EP of an expression cassette encoding IL-12. The CXCL9 and IL-12 can be expressed from a single expression cassette or plasmid or from multiple expression cassettes or plasmids. In some embodiments, for concurrent therapy, IT-EP CXCL9-IL12 therapy, CXCL9 and IL-12 are expressed from a single expression cassette or plasmid.

In some embodiments, intratumoral electroporation of an expression cassette encoding a CD3 half-BiTE can be administered with other therapeutic entities. In some embodiments, IT-EP CD3 half-BiTE therapy is combined IL-12 therapy. IL-12 therapy may occur before, concurrent with, and/or after IT-EP CD3 half-BiTE therapy. IL-12 therapy can occur before and concurrent with IT-EP CD3 half-BiTE therapy. IL-12 therapy can occur before and after IT-EP CD3 half-BiTE therapy. IL-12 therapy can occur concurrent with and after IT-EP CD3 half-BiTE therapy. IL-12 therapy may occur before, concurrent with, and after IT-EP CD3 half-BiTE therapy. IT-EP CD3 half-BiTE therapy may occur before, concurrent with, and/or after IL-12 therapy. IT-EP CD3 half-BiTE therapy may occur before and concurrent with IL-12 therapy. IT-EP CD3 half-BiTE therapy may occur before and after IL-12 therapy. IT-EP CD3 half-BiTE therapy may occur concurrent with and after IL-12 therapy. IT-EP CD3 half-BiTE therapy may occur before, concurrent with, and after IL-12 therapy. In some embodiments, IL-12 therapy is administered by IT-EP of an expression cassette encoding IL-12. The CD half-BiTE and IL-12 can be expressed from a single expression cassette or plasmid or from multiple expression cassettes or plasmids. In some embodiments, for concurrent therapy, IT-EP CD3 half-BiTE-IL12 therapy, CD3 half-BiTE and IL-12 are expressed from a single expression cassette or plasmid

In some embodiments, IT-EP CXCL9 therapy is combined with IT-EP CD3 half-BiTE therapy. In some embodiments, IT-EP CXCL9 and/or IT-EP CD3 half-BiTE therapy is combined with IL-12 therapy. IT-EP CD3 half-BiTE therapy may occur before, concurrent with, and/or after IT-EP CXCL9 therapy. IT-EP CD3 half-BiTE therapy can occur before and concurrent with IT-EP CXCL9 therapy. IT-EP CD3 half-BiTE therapy can occur before and after IT-EP CXCL9 therapy. IT-EP CD3 half-BiTE therapy can occur concurrent with and after IT-EP CXCL9 therapy. IT-EP CD3 half-BiTE therapy may occur before, concurrent with, and after IT-EP CXCL9 therapy. IT-EP CXCL9 therapy may occur before, concurrent with, and/or after IT-EP CD3 half-BiTE therapy. IT-EP CXCL9 therapy may occur before and concurrent with IT-EP CD3 half-BiTE therapy. IT-EP CXCL9 therapy may occur before and after IT-EP CD3 half-BiTE therapy. IT-EP CXCL9 therapy may occur concurrent with and after IT-EP CD3 half-BiTE therapy. IT-EP CXCL9 therapy may occur before, concurrent with, and after IT-EP CD3 half-BiTE therapy. Either CXCL3 or CD half-BiTE therapy can be combined with IL-12 therapy, such as by IT-EP of an expression cassette or plasmid encoding both CXCL9 and IL-12 or CD3-half-BiTe and IL-12, respectively (i.e., IT-EP IL12˜CXCL9 and IT-EP CD3 half-BiTE˜IL12 therapies).

In some embodiments, IT-EP CD3 half-BiTE therapy or IT-EP CD3 half-BiTE˜IL-12 therapy can be co-administered with one or more of IT-EP IL12 therapy, IT-EP CXCL9 therapy, and IT-EP IL12˜CXCL9 therapy.

In some embodiments, a described expression cassette is combined with one or more pharmaceutically acceptable excipients. Pharmaceutically acceptable excipients (excipients) are substances other than an active pharmaceutical ingredient (API, therapeutic product) that are intentionally included with the API (molecule). Excipients do not exert or are not intended to exert a therapeutic effect at the intended dosage. Excipients may act to a) aid in processing of the API during manufacture, b) protect, support or enhance stability, bioavailability or subject acceptability of the API, c) assist in product identification, and/or d) enhance any other attribute of the overall safety, effectiveness, of delivery of the API during storage or use. A pharmaceutically acceptable excipient may or may not be an inert substance. Excipients include, but are not limited to: absorption enhancers, anti-adherents, anti-foaming agents, anti-oxidants, binders, buffering agents, carriers, coating agents, colors, delivery enhancers, delivery polymers, dextran, dextrose, diluents, disintegrants, emulsifiers, extenders, fillers, flavors, glidants, humectants, lubricants, oils, polymers, preservatives, saline, salts, solvents, sugars, suspending agents, sustained release matrices, sweeteners, thickening agents, tonicity agents, vehicles, water-repelling agents, and wetting agents.

### VIII. Treatment Regimens/Cycles

The described IT-EP therapies can be administered at various intervals, depending upon such factors, for example, as the nature of the tumor, the condition of the subject, the size and chemical characteristics of the molecule and half-life of the molecule.

In some embodiments, methods for treating a tumor are described comprising, administering IT-EP IL12 therapy, followed by IT-EP CXCL9 and/or IT-EP IL12-CXCL9 therapy. IT-EP CXCL9 or IT-EP IL12-CXCL9 therapy can increase recruitment of tumor-specific T cells to the tumor or tumor microenvironment and/or increase activation of T cells. In some embodiments, IT-EP IL12 therapy is given to a tumor on day 0 (±1 day) and IT-EP CXCL9 therapy is given to the tumor on day 4 (±2 days) and day 7 (±2 days). In some embodiments, IT-EP IL12 therapy is given to a tumor on day 0 and IT-EP IL12-CXCL9 therapy is given to the tumor on day 4 (±2 days) and day 7 (±2 days).

In some embodiments, methods for treating a tumor are described comprising, administering IT-EP IL12 therapy, followed by IT-EP CD3 half-BiTE and/or CD3 half-BiTE˜IL12 therapy. In some embodiments, IT-EP IL12 therapy is given to a tumor on day 0 (±1 day) and IT-EP CD3 half-BiTE therapy is given to the tumor on day 4 (±2 days) and day 7 (±2 days). In some embodiments, IT-EP IL12 therapy is given to a tumor on day 0 and IT-EP CD3 half-BiTE˜IL12 therapy is given to the tumor on day 4 (±2 days) and day 7 (±2 days).

In some embodiments, methods for treating a tumor are described comprising, IT-EP IL12 therapy, following by IT-EP CXCL9 or IT-EP IL12˜CXCL9 therapy, and/or IT-EP CD3 half-BiTE or IT-EP CD3 half-BiTE˜IL-12 therapy.

In some embodiments, IT-EP IL12 therapy is administered first to increase tumor infiltrating lymphocytes. The tumor is subsequently treated with IT-EP CXCL9 or IL12˜CXCL9 therapy and/or IT-EP CD3 half-BiTE or IT-EP CD3 half-BiTE˜IL-12 therapy.

In some embodiments, IT-EP IL12˜CXCL9 therapy and/or IT-EP CD3 half-BiTE-IL-12 therapy are administered on day 0, days 0 and 4 (±2 days), days 0 and 7 (±2 days), or days 0, 4 (±2 days), and 7 (±2 days). In some embodiments, IT-EP IL12˜CXCL9 therapy is administered on day 0, days 0 and 4 (±2 days), days 0 and 7 (±2 days), or days 0, 4 (±2 days), and 7 (±2 days). In some embodiments, IT-EP CD3 half-BiTE˜IL-12 therapy is administered on day 0, days 1 and 4 (±2 days), days 1 and 7 (±2 days), or days 1, 4 (±2 days), and 7 (±2 days). In some embodiments, IT-EP IL12˜CXCL9 therapy and IT-EP CD3 half-BiTE˜IL-12 therapy are administered on day 0, days 0 and 4 (±2 days), days 0 and 7 (±2 days), or days 0, 4 (±2 days), and 7 (±2 days). Days 0, 4, and 7 are equivalent to days 1, 5, and 8.

A treatment cycle can comprise 1-6 IT-EP treatments. In some embodiments, a treatment cycle comprises 1, 2, or 3 IT-EP treatments. A cycle can be from about 1 week to about 6 weeks, or from about 2 weeks to about 5 weeks. In some embodiments, a cycle is about 3 weeks. In some embodiments, a cycle is about 6 weeks. In some embodiments, an IT-EP therapy is administered on one or more of days 0, 4 (±2 days), and 7 (±2 days) on alternating (every other) 3 week cycles (i.e., every 6 weeks).

In some embodiments, a cycle comprises 1-3 IT-EP treatments. The treatments can occur on days 1 (±2 days), 5 (±2 days) and/or day 8 (±2 days) (i.e., days 0 (±2 days), 4 (±2 days) and/or day 7 (±2 days)). Each treatment can comprise one or more of IT-EP IL2, IT-EP CXCL9, IT-EP IL12˜CXCL9, IT-EP CD3 half-BiTE, IT-EP CD3 half-BiTE˜IL12, and IT-EP anti-CTLA4 scFv.

In some embodiments, methods for treating a tumor are described comprising: administering IT-EP IL12 therapy on day 1 of a cycle and administering IT-EP CXCL9 or IT-EP IL12˜CXCL9 on days 5 (±2 days) and day 8 (±2 days) of the cycle. In some embodiments, methods for treating a tumor are described comprising: administering IT-EP IL12 therapy on day 1 of a cycle and administering IT-EP CD3 half-BiTE or IT-EP CD3 half-BiTE˜IL12 on days 5 (±2 days) and day 8 (±2 days) of the cycle. In some embodiments, methods for treating a tumor are described comprising: administering IT-EP IL12 therapy on day 1 of a cycle and administering one or more of IT-EP CXCL9 or IT-EP IL12˜CXCL9, IT-EP CD3 half-BiTE, and IT-EP CD3 half-BiTE˜IL12 on days 5 (±2 days) and day 8 (±2 days) of the cycle.

In some embodiments, methods for treating a tumor are described comprising: a) administering IT-EP IL12 therapy in a first cycle, b) administering IT-EP CXCL9 or IT-EP IL12˜CXCL9 therapy in a second cycle, and c) administering IT-EP CD3 half-BiTE or IT-EP CD3 half-BiTE˜IL-12 therapy in a third cycle. Each cycle can comprise 1-3 administrations of the corresponding IT-EP therapy.

Described are dosing regimens encompassing administering IT-EP IL12 therapy in combination IT-EP CXCL9 therapy and/or IT-EP CD3 half-BiTE therapy. Also described are dosing regimens encompassing administering IT-EP CXCL9 or IL12˜CXCL9 therapy with IT-EP CD3 half-BiTE or IT-EP CD3 half-BiTE˜IL12 therapy. The therapies may be administered concurrently, sequentially, or separately. In some embodiments, IT-EP IL12 therapy is administered in a first cycle and IT-EP CXCL9 therapy or IT-EP IL12CXCL9 therapy is administered in a second cycle. In some embodiments, IT-EP IL12 therapy is administered in a first cycle and IT-EP CD3 half-BiTE therapy or IT-EP CD3 half-BiTE-IL12 therapy is administered in a second cycle. In some embodiments, IT-EP 1112 therapy is administered in a first cycle, IT-EP CXCL9 therapy or IT-EP CXCL9-IL12 therapy is administered in a second cycle, and IT-EP CD3 half-BiTE therapy or IT-EP CD3 half-BiTE-IL12 therapy is administered in a third cycle. The 1T-EP therapy may be delivered on day 1 of each cycle. One or more of the cycles may be repeated as necessary. Within a cycle, the IT-EP therapy may be administered on a least one, two, or three days of the cycle. For example, a given expression cassette may be administered on day 1, day 5 (±2 days) and/or day 8 (±2 days).

In some embodiments, a CXCL9 or IL12˜CXCL9 plus IL-12 expression cassette is administered on days 1, 5±2, and 8±2 of a cycle. In some embodiments, a CTLA-4 scFv or anti-CTLA-4 scFv plus IL-12 expression cassette is administered on days 1, 5±2, and 8±2 of a cycle. In some embodiments, a CD3 half-BiTE or CD3 half-BiTE plus IL-12 expression cassette is administered on days 1, 5±2, and 8±2 of a cycle.

In some embodiments, a CXCL9 or CXCL9 plus IL-12 expression cassette (e.g., IL12˜CXCL9) is administered on days 1 and 5±2, and a CD3 half-BiTE or CD3 half-BiTE plus IL-12 expression cassette (e.g., CD3 half-BiTE˜IL12) is administered on day 8±2 of a cycle. In some embodiments, a CXCL9 or CXCL9 plus IL-12 expression cassette is administered on day 1, and a CD3 half-BiTE or CD3 half-BiTE plus IL-12 expression cassette is administered on days 5±2 and 8±2 of a cycle. In some embodiments, a CXCL9 or CXCL9 plus IL-12 expression cassette is administered on days 1 and 8±2, and a CD3 half-BiTE or CD3 half-BiTE plus IL-12 expression cassette is administered on day 5±2 of a cycle.

In some embodiments, a CD3 half-BiTE or CD3 half-BiTE plus IL-12 expression cassette is administered on days 1 and 5±2, and a CXCL9 or CXCL9 plus IL-12 expression cassette is administered on day 8±2 of a cycle. In some embodiments, a CD3 half-BiTE or CD3 half-BiTE plus IL-12 expression cassette is administered on days 1, and a CXCL9 or CXCL9 plus IL-12 expression cassette is administered on days 5±2 and 8±2 of a cycle. In some embodiments, a CD3 half-BiTE or CD3 half-BiTE plus IL-12 expression cassette is administered on days 1 and 8±2, and a CXCL9 or CXCL9 plus IL-12 expression cassette is administered on day 5±2 of a cycle.

In some embodiments, an IL-12-2A expression cassette is administered on day 1 and, and a CXCL9 or IL12˜CXCL9 expression cassette is administered on days 5±2 and 8±2 of a cycle. In some embodiments, an IL-12-2A expression cassette is administered on days 1 and 5±2, and a CXCL9 or IL12˜CXCL9 expression cassette is administered on day 8-2 of a cycle.

In some embodiments, an IL-12-2A expression cassette is administered on day 1 and, and a CD3 half-BiTE or CD3 half-BiTE˜IL-12 expression cassette is administered on days 5±2 and 8±2 of a cycle. In some embodiments, an IL-12-2A expression cassette is administered on days 1 and 5±2, and a CD3 half-BiTE or CD3 half-BiTE˜IL-12 expression cassette is administered on day 8±2 of a cycle.

In some embodiments, an IL12-2A expression cassette is administered on day 1, a CD3 half-BiTE or CD3 half-BiTE˜IL-12 expression cassette is administered on day 5±2, and a CXCL9 or IL12˜CXCL9 expression cassette is administered on day 8±2 of a cycle. In some embodiments, an IL-12-2A expression cassette is administered on day 1, a CXCL9 or IL12˜CXCL9 expression cassette is administered on day 5±2, and a CD3 half-BiTE or CD3 half-BiTE˜IL-12 expression cassette is administered on day 8±2 of a cycle.

In some embodiments, a subject is administered either IT-EP IL-12˜CXCL9 therapy or IT-EP CD3 half-BiTE˜IL12 therapy on days 0, 4 (±2 days), and 7(±2 days) provided the subject receives at least one IT-EP treatment with IL-12˜CXCL9 and one IT-EP treatment with CD3 half-BiTE˜IL12.

In some embodiments, a treatment can be administered every cycle or every other cycle. A cycle may be repeated such that 2 or more cycles are administered to a subject. Repeated cycles may be administered consecutively, alternated with one or more different cycles of treatment, or run concurrently with one or more difference cycles of treatment. Any of the above described treatments can be combined with other cancer therapies. For example, an IT-EP cycle can be combined with checkpoint inhibitor therapy.

### IX. Combination Therapy

In some embodiments, a therapeutic method includes a combination therapy. A combination therapy comprises a combination of therapeutic molecules or treatments. Therapeutic treatments include, but are not limited to, electric pulse (i.e., electroporation), radiation, antibody therapy, checkpoint inhibitor therapy, and chemotherapy. In some embodiments, administration of a combination therapy is achieved by electroporation alone. In some embodiments, administration of a combination therapy is achieved by a combination of electroporation and systemic delivery. In some embodiments, administration of a combination therapy is achieved by a combination of electroporation and radiation. In some embodiments, administration of a combination therapy is achieved by a combination of electroporation and oral medication. Therapeutic electroporation can be combined with, or administered with, one or more additional therapeutic treatments. The one or more additional therapeutics can be delivered by systemic delivery, intratumoral injection, intratumoral injection with electroporation, and/or radiation. The one or more additional therapeutics can be administered prior to, concurrent with, or subsequent to the CXCL9 and/or CD3 half-BiTE electroporation therapy.

In some embodiments, methods of treating cancer as described comprising: administering IT-EP therapy on day 1, days 1 and 5 (±2 days), days 1 and 8 (±2 days), or days 1, 5 (±2 days), and 8 (±2 days) and administering an additional therapeutic treatment on day 1 of a 3-6 week cycle. In some embodiments, methods of treating cancer as described comprising: administering IT-EP therapy on day 1, days 1 and 5 (±2 days), days 1 and 8 (±2 days), or days 1, 5 (±2 days), and 8 (±2 days) of every other cycle (i.e., every 6 weeks) and administering an additional therapeutic treatment on day 1 of each 3 week cycle (i.e., every 3 weeks). In some embodiments, the additional therapeutic treatment comprises a checkpoint inhibitor. In some embodiments, the additional checkpoint inhibitor therapy comprises anti-PD-1/anti-PD-L1 therapy. The checkpoint inhibitor therapy may be administered systemically.

### X. Electroporation (EP) Therapy

Electroporation therapy comprises administering at least one electroporative pulse to a cell, tissue, or tumor. As used herein electroporation therapy utilizes “reversible electroporation.” Reversible electroporation is the reversible, or temporary, permeabilization of cell membranes to molecules that are normally impermeable to the cell membranes using an electric pulse that is below the electric field threshold of the target cells. Because the electric pulse is below the cells' electric threshold, the cells can repair and are not killed by the electric pulse. Reversible electroporation can be used to delivery macromolecules, such as nucleic acid, into a cell without killing the cell. Reversible electroporation is a method that applies electric pulses to facilitate uptake of macromolecules, such as nucleic acids, into cells. Reversible electroporation has been used in several clinical trials to deliver DNA vaccines and has been shown to dramatically improve gene delivery (100-1000-fold) to cells in vivo.

Electroporation therapy can be performed using a known electroporation device suitable for use in a mammalian subject. The described expression cassettes can be administered to a subject before, during, or after administration of the electric pulse. The expression cassette can be administered at or near the tumor in a subject. The described expression cassettes can be injected into a tumor using a hypodermic needle.

In some embodiments, electroporation therapy comprises the administration of one or more voltage pulses. The nature of the electric field to be generated is determined by the nature of the tissue, the size of the selected tissue and its location. The voltage pulse that can be delivered to the tumor may be about 100 V/cm to about 1500V/cm. In some embodiments, the voltage pulse is about 700 V/cm to 1500 V/cm. In some embodiments, the voltage pulse may be about 600 V/cm, 650 V/cm, 700 V/cm, 750 V/cm, 800 V/cm, 850 V/cm, 900 V/cm, 950 V/cm, 1000 V/cm, 1050 V/cm, 1100 V/cm, 1150 V/cm, 1200 V/cm, 1250 V/cm, 1300 V/cm, 1350 V/cm, 1400 V/cm, 1450 V/cm, or 1500 V/cm. In some embodiments, the voltage pulse is 700±100. In some embodiments, the voltage pulse is 1300-1500 V/cm. In some embodiments, the voltage pulse is 1500±100 V/cm. In some embodiments, the voltage pulse is about 10 V/cm to 700 V/cm. In some embodiments, the voltage pulse is about 100 V/cm, 150 V/cm, 200 V/cm, 250 V/cm, 300 V/cm, 350 V/cm, or 400 V/cm, 450 V/cm, 500 V/cm, 550 V/cm, 600 V/cm, 650 V/cm, or 700 V/cm. In some embodiments, the voltage pulse is about 300 V/cm to about 500 V/cm. In some embodiments, the voltage pulse is 300-500 V/cm. In some embodiments, the voltage pulse is 350±50 V/cm.

The pulse duration of the electroporative pulse may be from 10 μsec to 1 second. In some embodiments, the pulse duration is from about 10 μsec to about 100 milliseconds (ms). In some embodiments, the pulse duration is from about 100 μsec to about 10 ms. In some embodiments, the pulse duration is 100 μsec, 1 ms, 5 ms, 10 ms, or 100 ms. The interval between pulses sets can be any desired time, such as one second. The waveform, electric field strength and pulse duration may also depend upon the type of cells and the type of molecules that are to enter the cells via electroporation.

The waveform of the electrical signal provided by the pulse generator can be an exponentially decaying pulse, a square pulse, a unipolar oscillating pulse train, a bipolar oscillating pulse train, or a combination of any of these forms. Square wave electroporation systems deliver controlled electric pulses that rise quickly to a set voltage, stay at that level for a set length of time (pulse length), and then quickly drop to zero.

1 to 100 pulses may be administered. In some embodiments, 1, 2, 3, 4, 5, 6, 7, 8, 9, or 10 pulses are administered. In some embodiments, 6 pulses are administered. In some embodiments, 6×0.1 msec pulses are administered. In some embodiments, 6×0.1 msec pulses are administered at 1300-1500 V/cm. In some embodiments 8 pulses are administered. In some embodiments 8×10 msec pulses are administered. In some embodiments 8×10 msec pulses are administered at 300-500 V/cm.

The electroporation device can comprise a single needle electrode, a pair of needle electrodes, or a plurality or array of needle electrodes. An array of needle electrodes can comprise 3, 4, 5, 6, 7, 8, 9, 10, or more electrodes. In some embodiments, the electroporation device can comprise a hypodermic needle or equivalent. In some embodiments, the electroporation device can comprise an electro-kinetic device (“EKD device”) able to produce a series of programmable constant-current pulse patterns between electrodes in an array based on user control and input of the pulse parameters.

Electroporation devices suitable for use with the described compounds, compositions, and methods include, but are not limited to, those described in U.S. Pat. Nos. 7,245,963, 5,439,440, 6,055,453, 6,009,347, 9,020,605, and 9,037,230, and U.S. Patent Publication Nos. 2005/0052630, 2019/0117964, and patent applications PCT/US2019/030437 and U.S. patent application Ser. No. 16/269,022.

“Intratumoral electroporation” comprises injecting a tumor, tumor microenvironment, and/or tumor margin tissue with an effective dose of a nucleic acid encoding a therapeutic polypeptide and administering electroporation therapy to the tumor, resulting in delivery of the nucleic acid into tumor cell and expression of the therapeutic polypeptide. The nucleic acid can be, but is not limited to, an expression vector, plasmid, or mRNA.

### List of Embodiments

1. A method of treating cancer in a subject, the method comprising:
(a) administering at least one dose of a checkpoint inhibitor and/or an immunostimulatory cytokine to the subject,
(b) obtaining a tumor sample from the subject;
(c) measuring CXCR3 expression in the tumor sample;
(d) determining whether CXCR3 expression is increased in the tumor sample relative to CXCR3 expression in a predetermined control; and
(e) if CXCR3 expression is increased in the tumor sample relative to CXCR3 expression in the predetermined control, then administering at least one additional dose of the checkpoint inhibitor and/or the immunostimulatory cytokine to the subject, or if CXCR3 expression is not increased in the tumor sample relative to CXCR3 expression in the predetermined control, then administering at least one pharmaceutically effective dose of CXCL9 and/or CD3 half-BiTE and at least one additional dose of the checkpoint inhibitor and/or the immunostimulatory cytokine to the subject.

2. The method of embodiment 1, wherein step (a) comprises administering at least one dose of the checkpoint inhibitor wherein the checkpoint inhibitor is administered systemically.

3. The method of embodiment 2, wherein the checkpoint inhibitor comprises an anti-PD-1 or anti-PD-L1 antibody.

4. The method of embodiment 3, wherein the checkpoint inhibitor comprises nivolumab, pembrolizumab, pidilizumab, or atezolizumab.

5. The method of any one of embodiments 1-4, wherein step (a) comprises administering at least one dose of the immunostimulatory cytokine wherein the immunostimulatory cytokine is administered by intratumoral electroporation of a nucleic acid encoding the immunostimulatory cytokine.

6. The method of embodiment 5, wherein the immunostimulatory cytokine comprises IL-12 or IL-15.

7. The method of embodiment 6, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

8. The method of embodiment 1, wherein step (a) comprises administering at least one dose of a checkpoint inhibitor and at least one dose of an immunostimulatory cytokine, wherein the checkpoint inhibitor comprises an anti-PD-1 or anti-PD-L1 antibody administered systemically, and the immunostimulatory cytokine comprises IL-12 administered by intratumoral electroporation of a nucleic acid encoding the IL-12.

9. The method of any one of embodiments 1-8, wherein measuring CXCR3 expression in the tumor sample comprises measuring CXCR3 mRNA in the tumor sample.

10. The method of embodiment 9, wherein measuring CXCR3 mRNA comprises performing a quantitative polymerase chain reaction.

11. The method of any one of embodiments 1-8, wherein measuring CXCR3 expression in the tumor sample comprises measuring CXCR3 protein in the tumor sample.

12. The method of any one of embodiments 1-8, wherein measuring CXCR3 expression in the tumor sample comprises measuring a number of CXCR3⁺ T cells in the tumor sample.

13. The method of any one of embodiments 1-12, wherein the predetermined control comprises a tumor sample obtained from the subject prior to step (a).

14. The method of any one of embodiments 1-12, wherein the predetermined control comprises a standard derived from a population of known responders and/or known non-responders to checkpoint inhibitor and/or an immunostimulatory cytokine therapy.

15. The method of any one of embodiments 1-14, wherein administering at least one pharmaceutically effective dose of CXCL9 and/or CD3 half-BiTE comprises intratumoral electroporation of a nucleic acid encoding CXCL9 and/or CD3 half-BiTE.

16. The method of embodiment 15, wherein the nucleic acid encoding CXCL9 and/or CD3 half-BiTE further encodes the immunostimulatory cytokine, wherein the immunostimulatory cytokine comprises IL-12.

17. The method of any one of embodiments 1-14, wherein administering at least one additional dose of the checkpoint inhibitor and/or the immunostimulatory cytokine comprises: administering at least one additional dose of the checkpoint inhibitor, administering at least one additional dose of the immunostimulatory cytokine, or administering at least one additional dose of the checkpoint inhibitor and the immunostimulatory cytokine.

18. The method of embodiment 17, wherein the checkpoint inhibitor comprises an anti-PD-1 or anti-PD-L1 antibody administered systemically.

19. The method of embodiment 17, wherein the immunostimulatory cytokine comprises IL-12 administered by intratumoral electroporation of a nucleic acid encoding the IL-12.

20. The method of embodiment 19, wherein the nucleic acid encoding the IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

21. The method of any one of embodiments 1-20, wherein the subject is a human.

22. A method of treating cancer in a subject, comprising:
(a) administering at least one dose of a checkpoint inhibitor and/or an immunostimulatory cytokine to the subject;
(b) measuring a level of CXCR3 in a tumor sample obtained from the subject after the step of administering the checkpoint inhibitor and/or the immunostimulatory cytokine; and
(c) administering to the subject at least one pharmaceutically effective dose of CXCL9 and/or CD3 half-BiTE if the level of CXCR3 in the tumor sample is not increased relative to the level of CXCR3 in a predetermined control.

23. The method of embodiment 22, wherein step (a) comprises administering at least one dose of the checkpoint inhibitor wherein the checkpoint inhibitor is administered systemically.

24. The method of embodiment 23, wherein the checkpoint inhibitor comprises an anti-PD-1 or anti-PD-L1 antibody.

25. The method of embodiment 24, wherein the checkpoint inhibitor comprises nivolumab, pembrolizumab, pidilizumab, or atezolizumab.

26. The method of any one of embodiments 22-25, wherein step (a) comprises administering at least one dose of the immunostimulatory cytokine wherein the immunostimulatory cytokine is administered by intratumoral electroporation of a nucleic acid encoding the immunostimulatory cytokine.

27. The method of embodiment 26, wherein the immunostimulatory cytokine comprises IL-12 or IL-15.

28. The method of embodiment 27, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

29. The method of embodiment 22, wherein step (a) comprises administering at least one dose of a checkpoint inhibitor and at least one dose of an immunostimulatory cytokine, wherein the checkpoint inhibitor comprises an anti-PD-1 or anti-PD-L1 antibody administered systemically, and the immunostimulatory cytokine comprises IL-12 administered by intratumoral electroporation of a nucleic acid encoding the IL-12.

30. The method of any one of embodiments 22-29, wherein measuring CXCR3 expression in the tumor sample comprises measuring CXCR3 mRNA in the tumor sample.

31. The method of embodiment 29, wherein measuring CXCR3 mRNA comprises performing a quantitative polymerase chain reaction.

32. The method of any one of embodiments 22-29, wherein measuring CXCR3 expression in the tumor sample comprises measuring CXCR3 protein in the tumor sample.

33. The method of any one of embodiments 22-29, wherein measuring CXCR3 expression in the tumor sample comprises measuring a number of CXCR3⁺ T cells in the tumor sample.

34. The method of any one of embodiments 22-33, wherein the predetermined control comprises a tumor sample obtained from the subject prior to step (a).

35. The method of any one of embodiments 22-33, wherein the predetermined control comprises a standard derived from a population of known responders and/or known non-responders to checkpoint inhibitor and/or an immunostimulatory cytokine therapy.

36. The method of any one of embodiments 22-35, wherein administering at least one pharmaceutically effective dose of CXCL9 and/or CD3 half-BiTE comprises intratumoral electroporation of a nucleic acid encoding CXCL9 and/or CD3 half-BiTE.

37. The method of embodiment 36, wherein the nucleic acid encoding CXCL9 and/or CD3 half-BiTE further encodes the immunostimulatory cytokine, wherein the immunostimulatory cytokine comprises IL-12.

38. The method of embodiment 22, wherein administering at least one dose of the checkpoint inhibitor and/or the immunostimulatory cytokine comprises: administering at least one dose of the checkpoint inhibitor, administering at least one dose of the immunostimulatory cytokine, or administering at least one dose of the checkpoint inhibitor and the immunostimulatory cytokine.

39. The method of embodiment 38, wherein the checkpoint inhibitor comprises an anti-PD-1 or anti-PD-L1 antibody administered systemically.

40. The method of embodiment 38, wherein the immunostimulatory cytokine comprises IL-12 administered by intratumoral electroporation of a nucleic acid encoding the IL-12.

41. The method of embodiment 40, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

42. The method of any one of embodiments 22-41, wherein the subject is a human.

43. A method of identifying a subject with cancer at risk of not responding to checkpoint inhibitor and/or immunostimulatory cytokine therapy, the method comprising: measuring a level of CXCR3 in a tumor sample obtained from the subject that has been administered at least one dose of a checkpoint inhibitor and/or an immunostimulatory cytokine, wherein the level of CXCR3 in the tumor sample less than a predetermined control indicates the subject is at risk of not responding to the checkpoint inhibitor and/or immunostimulatory cytokine therapy.

44. The method of embodiment 43, wherein the checkpoint inhibitor comprises an anti-PD-1 or anti-PD-L1 antibody.

45. The method of embodiment 44, wherein the checkpoint inhibitor comprises nivolumab, pembrolizumab, pidilizumab, or atezolizumab.

46. The method of embodiment 43, wherein the immunostimulatory cytokine comprises IL-12 or IL-15.

47. The method of any one of embodiments 43-46, wherein measuring the level of CXCR3 in the tumor sample comprises measuring CXCR3 mRNA in the tumor sample.

48. The method of embodiment 47, wherein measuring CXCR3 mRNA comprises performing a quantitative polymerase chain reaction.

49. The method of any one of embodiments 43-46, wherein measuring the level of CXCR3 in the tumor sample comprises measuring CXCR3 protein in the tumor sample.

50. The method of any one of embodiments 43-46, wherein measuring the level of CXCR3 in the tumor sample comprises measuring a number of CXCR3⁺ T cells in the tumor sample.

51. The method of any one of embodiments 43-50, wherein the predetermined control comprises a tumor sample obtained from the subject prior to the subject being administered the at least one dose of the checkpoint inhibitor and/or the immunostimulatory cytokine.

52. The method of any one of embodiments 43-50, wherein the predetermined control comprises a standard derived from a population of known responders and/or known non-responders to checkpoint inhibitor and/or an immunostimulatory cytokine therapy.

53. The method of any one of embodiments 43-52, wherein the subject is a human.

54. A method of treating cancer in a subject comprising:
(a) identifying whether the subject is at risk of not responding to checkpoint inhibitor and/or immunostimulatory cytokine therapy by
   (i) administering at least one dose of a checkpoint inhibitor and/or an immunostimulatory cytokine to the subject, and
   (ii) measuring a level of CXCR3 in a tumor sample obtained from the subject after the step of administering the at least one dose of the checkpoint inhibitor and/or the immunostimulatory cytokine,
   wherein the level of CXCR3 in the tumor sample less than a predetermined control indicates the subject is at risk of not responding to the checkpoint inhibitor and/or immunostimulatory cytokine therapy; and
(b) administering to the subject identified as being at risk of not responding to checkpoint inhibitor and/or immunostimulatory cytokine therapy at least one pharmaceutically effective dose of CXCL9 and/or CD-3 half-BiTE.

55. The method of embodiment 54, wherein step (a) comprises administering at least one dose of the checkpoint inhibitor wherein the checkpoint inhibitor is administered systemically.

56. The method of embodiment 55, wherein the checkpoint inhibitor comprises an anti-PD-1 or anti-PD-L1 antibody.

57. The method of embodiment 56, wherein the checkpoint inhibitor comprises nivolumab, pembrolizumab, pidilizumab, or atezolizumab.

58. The method of any one of embodiments 54-57, wherein step (a) comprises administering at least one dose of the immunostimulatory cytokine wherein the immunostimulatory cytokine is administered by intratumoral electroporation of a nucleic acid encoding the immunostimulatory cytokine.

59. The method of embodiment 58, wherein the immunostimulatory cytokine comprises IL-12 or IL-15.

60. The method of embodiment 59, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

61. The method of embodiment 1, wherein step (a) comprises administering at least one dose of a checkpoint inhibitor and at least one dose of an immunostimulatory cytokine, wherein the checkpoint inhibitor comprises an anti-PD-1 or anti-PD-L1 antibody administered systemically, and the immunostimulatory cytokine comprises IL-12 administered by intratumoral electroporation of a nucleic acid encoding the IL-12.

62. The method of any one of embodiments 54-60, wherein measuring the level of CXCR3 in the tumor sample comprises measuring CXCR3 mRNA in the tumor sample.

63. The method of embodiment 62, wherein measuring CXCR3 mRNA comprises performing a quantitative polymerase chain reaction.

64. The method of any one of embodiments 54-61, wherein measuring the level of CXCR3 in the tumor sample comprises measuring CXCR3 protein in the tumor sample.

65. The method of any one of embodiments 54-61, wherein measuring the level of CXCR3 in the tumor sample comprises measuring a number of CXCR3⁺ T cells in the tumor sample.

66. The method of any one of embodiments 54-65, wherein the predetermined control comprises a tumor sample obtained from the subject prior to the administering at least one dose of the checkpoint inhibitor and/or the immunostimulatory cytokine to the subject.

67. The method of any one of embodiments 54-65, wherein the predetermined control comprises a standard derived from a population of known responders and/or known non-responders to checkpoint inhibitor and/or an immunostimulatory cytokine therapy.

68. The method of any one of embodiments 54-67, wherein administering at least one pharmaceutically effective dose of CXCL9 and/or CD3 half-BiTE comprises intratumoral electroporation of a nucleic acid encoding CXCL9 and/or CD3 half-BiTE.

69. The method of embodiment 68, wherein the nucleic acid encoding CXCL9 and/or CD3 half-BiTE further encodes the immunostimulatory cytokine, wherein the immunostimulatory cytokine comprises IL-12.

70. The method of embodiment 54-67, wherein administering at least one dose of the checkpoint inhibitor and/or the immunostimulatory cytokine comprises: administering at least one dose of the checkpoint inhibitor, administering at least one dose of the immunostimulatory cytokine, or administering at least one dose of the checkpoint inhibitor and the immunostimulatory cytokine.

71. The method of embodiment 70, wherein the checkpoint inhibitor comprises an anti-PD-1 or anti-PD-L1 antibody administered systemically.

72. The method of embodiment 70, wherein the immunostimulatory cytokine comprises IL-12 administered by intratumoral electroporation of a nucleic acid encoding the IL-12.

73. The method of embodiment 72, wherein the nucleic acid encoding the IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

74. The method of any one of embodiments 54-73, wherein the subject is a human.

75. A method for treating a patient with cancer, the method comprising:
(a) obtaining a tumor sample from the patient,
(b) measuring a level of CXCR3 expression in the tumor sample,
(c) correlating the level of CXCR3 expression in the tumor sample with a reference level obtained from a predetermined control or standard derived from a population of known responders and/or known non-responders to determine whether the patient is at risk of progressing on checkpoint inhibitor therapy, and
(d) if the expression level is greater than the reference level, then administering at least one dose of a checkpoint inhibitor and/or an immunostimulatory cytokine, or if the expression level is less than the reference level then administering to the patient at least one pharmaceutically effective dose of CXCL9 and/or CD3 half-BiTE and at least one dose of the checkpoint inhibitor and/or the immunostimulatory cytokine.

76. The method of embodiment 75, wherein measuring CXCR3 expression in the tumor sample comprises measuring CXCR3 mRNA in the tumor sample.

77. The method of embodiment 76, wherein measuring CXCR3 mRNA comprises performing a quantitative polymerase chain reaction.

78. The method of embodiment 75, wherein measuring CXCR3 expression in the tumor sample comprises measuring CXCR3 protein in the tumor sample.

79. The method of embodiment 75, wherein measuring CXCR3 expression in the tumor sample comprises measuring a number of CXCR3⁺ T cells in the tumor sample.

80. The method of any one of embodiments 75-79, wherein administering at least one dose of the checkpoint inhibitor and/or the immunostimulatory cytokine comprises: administering at least one dose of the checkpoint inhibitor, administering at least one dose of the immunostimulatory cytokine, or administering at least one dose of the checkpoint inhibitor and the immunostimulatory cytokine.

81. The method of embodiment 80, wherein the checkpoint inhibitor is administered systemically.

82. The method of embodiment 81, wherein the checkpoint inhibitor comprises an anti-PD-1 or anti-PD-L1 antibody.

83. The method of embodiment 82, wherein the checkpoint inhibitor comprises nivolumab, pembrolizumab, pidilizumab, or atezolizumab.

84. The method of any one of embodiments 80, wherein administering at least one dose of the immunostimulatory cytokine comprises intratumoral electroporation of a nucleic acid encoding the immunostimulatory cytokine.

85. The method of embodiment 84, wherein the immunostimulatory cytokine comprises IL-12 or IL-15.

86. The method of embodiment 85, wherein the nucleic acid encoding the IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

87. The method of any one of embodiments 75-86, wherein administering at least one pharmaceutically effective dose of CXCL9 and/or CD3 half-BiTE comprises intratumoral electroporation of a nucleic acid encoding CXCL9 and/or CD3 half-BiTE.

88. The method of embodiment 87, wherein the nucleic acid encoding CXCL9 and/or CD3 half-BiTE further encodes the immunostimulatory cytokine, wherein the immunostimulatory cytokine comprises IL-12.

89. The method of embodiment 88, wherein the IL-12 and the CXCL9 and/or CD3 half-BiTE are expressed from a single promoter.

90. The method of any one of embodiments 75-89, wherein the subject is a human.

91. A nucleic acid encoding IL-12 for use in a method of treating a subject with cancer, wherein the method comprises:
(a) administering at least one dose of a checkpoint inhibitor and/or an immunostimulatory cytokine to the subject,
(b) obtaining a tumor sample from the subject;
(c) measuring CXCR3 expression in the tumor sample;
(d) determining whether CXCR3 expression is increased in the tumor sample relative to CXCR3 expression in a predetermined control; and
(e) if CXCR3 expression is increased in the tumor sample relative to CXCR3 expression in the predetermined control, then administering at least one dose of the nucleic acid encoding IL-12 by intratumoral electroporation, or if CXCR3 expression is not increased in the tumor sample relative to CXCR3 expression in the predetermined control, then administering to the subject at least one pharmaceutically effective dose of a nucleic acid encoding CXCL9 and/or CD3 half-BiTE by intratumoral electroporation and at least one dose of the nucleic acid encoding IL-12 by intratumoral electroporation.

92. A method of treating cancer in a subject, the method comprising:
(a) obtaining a tumor sample from the subject;
(b) measuring CXCR3 expression in the tumor sample;
(c) determining whether CXCR3 expression is increased in the tumor sample relative to CXCR3 expression in a predetermined control; and
(d) if CXCR3 expression is increased in the tumor sample relative to CXCR3 expression in the predetermined control, then administering at least one dose of the checkpoint inhibitor and/or an immunostimulatory cytokine to the subject, or if CXCR3 expression is not increased in the tumor sample relative to CXCR3 expression in the predetermined control, then administering at least one pharmaceutically effective dose of CXCL9 and/or CD3 half-BiTE and at least one dose of a checkpoint inhibitor and/or an immunostimulatory cytokine to the subject.

93. The method of embodiment 92, wherein measuring CXCR3 expression in the tumor sample comprises measuring CXCR3 mRNA in the tumor sample.

94. The method of embodiment 93, wherein measuring CXCR3 mRNA comprises performing a quantitative polymerase chain reaction.

95. The method of embodiment 92, wherein measuring CXCR3 expression in the tumor sample comprises measuring CXCR3 protein in the tumor sample.

96. The method of embodiment 92, wherein measuring CXCR3 expression in the tumor sample comprises measuring a number of CXCR3⁺ T cells in the tumor sample.

97. The method of any one of embodiments 92-96, wherein the predetermined control comprises a standard derived from a population of known responders and/or known non-responders to checkpoint inhibitor and/or an immunostimulatory cytokine therapy.

98. The method of embodiment 97, wherein the checkpoint inhibitor comprises and anti-PD-1 or anti-PD-L1 antibody.

99. The method of any one of embodiments 92-98, wherein administering at least one dose of the checkpoint inhibitor and/or the immunostimulatory cytokine comprises: administering at least one dose of the checkpoint inhibitor, administering at least one dose of the immunostimulatory cytokine, or administering at least one dose of the checkpoint inhibitor and the immunostimulatory cytokine.

100. The method of embodiment 99, wherein the checkpoint inhibitor is administered systemically.

101. The method of embodiment 100, wherein the checkpoint inhibitor comprises an anti-PD-1 or anti-PD-L1 antibody.

102. The method of embodiment 101, wherein the checkpoint inhibitor comprises nivolumab, pembrolizumab, pidilizumab, or atezolizumab.

103. The method of any one of embodiments 99, wherein administering at least one dose of the immunostimulatory cytokine comprises intratumoral electroporation of a nucleic acid encoding the immunostimulatory cytokine.

104. The method of embodiment 103, wherein the immunostimulatory cytokine comprises IL-12 or IL-15.

105. The method of embodiment 104, wherein the nucleic acid encoding the IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

106. The method of any one of embodiments 92-105, wherein administering at least one pharmaceutically effective dose of CXCL9 and/or CD3 half-BiTE comprises intratumoral electroporation of a nucleic acid encoding CXCL9 and/or CD3 half-BiTE.

107. The method of embodiment 106, wherein the nucleic acid encoding CXCL9 and/or CD3 half-BiTE further encodes the immunostimulatory cytokine, wherein the immunostimulatory cytokine comprises IL-12.

108. The method of any one of embodiments 92-107-20, wherein the subject is a human.

109. A method of identifying a subject with cancer at risk of not responding to checkpoint inhibitor and/or immunostimulatory cytokine therapy, the method comprising: measuring a level of CXCR3 in a tumor sample obtained from the subject, wherein the level of CXCR3 in the tumor sample less than a predetermined control indicates the subject is at risk of not responding to the checkpoint inhibitor and/or immunostimulatory cytokine therapy.

110. The method of embodiment 109, wherein measuring CXCR3 expression in the tumor sample comprises measuring CXCR3 mRNA in the tumor sample.

111. The method of embodiment 100, wherein measuring CXCR3 mRNA comprises performing a quantitative polymerase chain reaction.

112. The method of embodiment 109, wherein measuring CXCR3 expression in the tumor sample comprises measuring CXCR3 protein in the tumor sample.

113. The method of embodiment 109, wherein measuring CXCR3 expression in the tumor sample comprises measuring a number of CXCR3⁺ T cells in the tumor sample.

114. The method of any one of embodiments 102-113, wherein the predetermined control comprises a standard derived from a population of known responders and/or known non-responders to checkpoint inhibitor and/or an immunostimulatory cytokine therapy.

115. The method of any one of embodiments 1-42, 54-114 wherein administering at least one pharmaceutically effective dose of CXCL9 and/or CD-3 half-BiTE results in increased numbers of CXCR3⁺ T cells in the tumor.

TABLE 1
Sequences (nucleotides or amino acids in parenthesis may or may not be present)

Igκ signal peptide nucleic acid sequence (SEQ ID NO: 1)
atggagacagacacactcctgctatgggtactgctgctctgggttccaggttccactggtgac
Igκ signal peptide amino acid sequence (SEQ ID NO: 2)
METDTLLLWVLLLWVPGSTGD
HA tag nucleic acid sequence (SEQ ID NO: 3)
tatccatatgatgttccagattatgct
HA tag amino acid sequence (SEQ ID NO: 4)
YPYDVPDYA
Myc tag nucleic acid sequence (SEQ ID NO: 5)
gaacaaaaactcatctcagaagaggatctg
Myc tag amino acid sequence (SEQ ID NO: 6)
EQKLISEEDL
2C11 Variable heavy chain nucleic acid sequence (SEQ ID NO: 7)
gaggtgcagctggtggagtctgggggaggcttggtgcagcctggaaagtccctgaaactctcctgtgaggcct
ctggattcaccttcagcggctatggcatgcactgggtccgccaggctccagggagggggctggagtcggtcgc
atacattactagtagtagtattaatatcaaatatgctgacgctgtgaaaggccggttcaccgtctccagagac
aatgccaagaacttactgtttctacaaatgaacattctcaagtctgaggacacagccatgtactactgtgcaa
gattcgactgggacaaaaattactggggccaaggaaccatggtcaccgtctcctcaggtggcggt
2C11 Variable heavy chain amino acid sequence (SEQ ID NO: 8)
EVQLVESGGGLVQPGKSLKLSCEASGFTFSGYGMHWVRQAPGRGLESVAYITSSSINIKYADAVKGRFTVSRD
NAKNLLFLQMNILKSEDTAMYYCARFDWDKNYWGQGTMVTVSSGGG
2C11 Variable light chain nucleic acid sequence (SEQ ID NO: 9)
atgacccagtctccatcatcactgcctgcctccctgggagacagagtcactatcaattgtcaggccagtcagg
acattagcaattatttaaactggtaccagcagaaaccagggaaagctcctaagctcctgatctattatacaaa
taaattggcagatggagtcccatcaaggttcagtggcagtggttctgggagagattcttctttcactatcagc
agcctggaatccgaagatattggatcttattactgtcaacagtattataactatccgtggacgttcggacctg
gcaccaagctggaaatcaaa
2C11 Variable light chain nucleic acid sequence (SEQ ID NO: 10)
atgacccagtctccatcatcactgcctgcctccctgggagacagagtcactatcaattgtcaggccagtcagg
acattagcaattatttaaactggtatcagcagaaaccagggaaagctcctaagctcctgatctattatacaaa
taaattggcagatggagtcccatcaaggttcagtggcagtggttctgggagagattcttctttcactatcagc
agcctggaatccgaagatattggatcttattactgtcaacagtattataactatccgtggacgttcggacctg
gcaccaagctggaaatcaaa
2C11 Variable light chain amino acid sequence (SEQ ID NO: 11)
MTQSPSSLPASLGDRVTINCQASQDISNYLNWYQQKPGKAPKLLIYYTNKLADGVPSRFSGSGSGRDSSFTIS
SLESEDIGSYYCQQYYNYPWTFGPGTKLEIK
Linker nucleic acid sequence (SEQ ID NO: 12)
agtggctctggagggggctctggggggattgggggggg
Linker amino acid sequence (SEQ ID NO: 13
SGSGGGSGGGSGGGS
Linker nucleic acid sequence (SEQ ID NO: 14)
ggtggcggtggctccggcggtggtgggtcgggtggcggcggatct
Linker amino acid sequence (SEQ ID NO: 15)
GGGGSGGGGSGGGGS
Linker nucleic acid sequence (SEQ ID NO: 16)
ggctccggcggtggtgggtcgggggggggatt
Linker amino acid sequence (SEQ ID NO: 17)
GSGGGGSGGGG
Linker nucleic acid sequence (SEQ ID NO: 18)
ggcagtgggagtgggagtgggagtggg
Linker amino acid sequence (SEQ ID NO: 19)
GSGSGSGSG
Linker nucleic acid sequence (SEQ ID NO: 20)
ggcagtgggagtggg
Linker amino acid sequence (SEQ ID NO: 21)
GSGSG
Linker nucleic acid sequence (SEQ ID NO: 22)
tctagtggatccggt
Linker amino acid sequence (SEQ ID NO: 23)
SSGSG
PDGFR TM segment nucleic acid sequence (SEQ ID NO: 24)
gtgggccaggacacgcaggaggtcatcgtggtgccacactccttgccctttaaggtggtggtgatctcagcca
tcctggccctggtggtgctcaccatcatctcccttatcatcctcatcatgctttggcagaagaagccacgt
PDGFR TM segment amino acid sequence (SEQ ID NO: 25)
VGQDTQEVIVVPHSLPFKVVVISAILALVVLTIISLIILIMLWQKKPR
PDGFR TM segment nucleic acid sequence (SEQ ID NO: 26)
gctgtgggccaggacacgcaggaggtcatcgtggtgccacactccttgccctttaaggtggtggtgatctcag
ccatcctggccctggtggtgctcaccatcatctcccttatcatcctcatcatgctttggcagaagaagccacg
t
PDGFR TM segment amino acid sequence (SEQ ID NO: 27)
AVGQDTQEVIVVPHSLPFKVVVISAILALVVLTIISLIILIMLWQKKPR
P2A nucleic acid sequence (SEQ ID NO: 28)
ggatctggggccaccaacttttcattgctcaagcaggcgggcgatgtggaggaaaaccctggcccc
P2A amino acid sequence (SEQ ID NO: 29)
GSGATNFSLLKQAGDVEENPGP
mIL12-p35 nucleic acid sequence (SEQ ID NO: 30)
(ggtacc or atg)gtcagcgttccaacagcctcaccctcggcatccagcagctcctctcagtgccggtcc
agcatgtgtcaatcacgctacctcctctttttggccacccttgccctcctaaaccacctcagtttggccaggg
tcattccagtctctggacctgccaggtgtcttagccagtcccgaaacctgctgaagaccacagatgacatggt
gaagacggccagagaaaaactgaaacattattcctgcactgctgaagacatcgatcatgaagacatcacacgg
gaccaaaccagcacattgaagacctgtttaccactggaactacacaagaacgagagttgcctggctactagag
agacttcttccacaacaagagggagctgcctgcccccacagaagacgtctttgatgatgaccctgtgccttgg
tagcatctatgaggacttgaagatgtaccagacagagttccaggccatcaacgcagcacttcagaatcacaac
catcagcagatcattctTgacaagggcatgctggtggccatcgatgagctgatgcagtctctgaatcataatg
gcgagactctgcgccagaaacctcctgtgggagaagcagacccttacagagtgaaaatgaagctctgcatcct
gcttcacgccttcagcacccgcgtcgtgaccatcaacagggtgatgggctatctgagctccgcc
mIL12-35 amino acid sequence (SEQ ID NO: 31)
(M)VSVPTASPSASSSSSQCRSSMCQSRYLLFLATLALLNHLSLARVIPVSGPARCLSQSRNLLKTTDDMVKT
AREKLKHYSCTAEDIDHEDITRDQTSTLKTCLPLELHKNESCLATRETSSTTRGSCLPPQKTSLMMTLCLGSI
YEDLKMYQTEFQAINAALQNHNHQQIILDKGMLVAIDELMQSLNHNGETLRQKPPVGEADPYRVKMKLCILLH
AFSTRVVTINRVMGYLSSAAAA
mIL12-p40 nucleic acid sequence (SEQ ID NO: 32)
tgtcctcagaagctaaccatctcctggtttgccatcgttttgctggtgtctccactcatggccatgtgggagc
tggagaaagacgtttatgttgtagaggtggactggactcccgatgcccctggagaaacagtgaacctcacctg
tgacacgcctgaagaagatgacatcacctggacctcagaccagagacatggagtcataggctctggaaagacc
ctgaccatcactgtcaaagagtttctTgatgctggccagtacacctgccacaaaggaggcgagactctgagcc
actcacatctgctgctccacaagaaggaaaatggaatttggtccactgaaattttaaaGaatttcaaGaacaa
gactttcctgaagtgtgaagcaccaaattactccggacggttcacgtgctcatggctggtgcaaagaaacatg
gacttgaagttcaacatcaagagcagtagcagttcccctgactctcgggcagtgacatgtggaatggcgtctc
tgtctgcagagaaggtcacactggaccaaagggactatgagaagtattcagtgtcctgccaggaggatgtcac
ctgcccaactgccgaggagaccctgcccattgaactggcgttggaagcacggcagcagaataaatatgagaac
tacagcaccagcttcttcatcagggacatcatcaaaccagacccgcccaagaacttgcagatgaagcctttga
agaactcacaggtggaggtcagctgggagtaccctgactcctggagcactccccattcctacttctccctcaa
gttctttgttcgaatccagcgcaagaaagaaaagatgaaggagacagaggaggggtgtaaccagaaaggtgcg
ttcctcgtagagaagacatctaccgaagtccaatgcaaaggcgggaatgtctgcgtgcaagctcaggatcgct
attacaattcctcatgcagcaagtgggcatgtgttccctgcagggtccgatcc (tag or tcg)
mIL12-p40 amino acid sequence (SEQ ID NO: 33)
CPQKLTISWFAIVLLVSPLMAMWELEKDVYVVEVDWTPDAPGETVNLTCDTPEEDDITWTSDQRHGVIGSGKT
LTITVKEFLDAGQYTCHKGGETLSHSHLLLHKKENGIWSTEILKNFKNKTFLKCEAPNYSGRFTCSWLVQRNM
DLKFNIKSSSSSPDSRAVTCGMASLSAEKVTLDQRDYEKYSVSCQEDVTCPTAEETLPIELALEARQQNKYEN
YSTSFFIRDIIKPDPPKNLQMKPLKNSQVEVSWEYPDSWSTPHSYFSLKFFVRIQRKKEKMKETEEGCNQKGA
FLVEKTSTEVQCKGGNVCVQAQDRYYNSSCSKWACVPCRVRS(S)
mCXCL9 nucleic acid sequence (SEQ ID NO: 34)
(atg)aagtccgctgttcttttcctcttgggcatcatcttcctggagcagtgtggagttcgaggaaccctagt
gataaggaatgcacgatgctcctgcatcagcaccagccgaggcacgatccactacaaatccctcaaagacctc
aaacagtttgccccaagccccaattgcaacaaaactgaaatcattgctacactgaagaacggagatcaaacct
gcctagatccggactcggcaaatgtgaagaagctgatgaaagaatgggaaaagaagatcagccaaaagaaaaa
gcaaaagagggggaaaaaacatcaaaagaacatgaaaaacagaaaacccaaaacaccccaaagtcgtcgtcgt
tcaaggaagactaca(taa)
mCXCL9 amino acid sequence (SEQ ID NO: 35)
(M)KSAVLFLLGIIFLEQCGVRGTLVIRNARCSCISTSRGTIHYKSLKDLKQFAPSPNCNKTEIIATLKNGDQ
TCLDPDSANVKKLMKEWEKKISQKKKQKRGKKHQKNMKNRKPKTPQSRRRSRKTT
Mouse anti-CTLA-4 9D9 variable light chain nucleic acid sequence (SEQ ID NO: 36)
gacattgtgatgacacagaccacactcagtctccccgtttcccttggtgatcaagcctccatatcctgtaggt
ctagtcaatctatcgtccactccaacggcaatacctatctggaatggtatcttcaaaagcccggacaatcacc
aaagcttcttatctataaggtgagcaatagatttagcggggtccctgaccgattctctggaagtggctctggc
acagactttaccttgaaaatctccagagttgaggctgaggaccttggtgtatactactgcttccaaggctctc
atgttccctacactttcggaggcggaacaaaactggagataaaacgagccgacgcagcccccactgtg
Mouse anti-CTLA-4 9D9 variable light chain amino acid sequence (SEQ ID NO: 37)
DIVMTQTTLSLPVSLGDQASISCRSSQSIVHSNGNTYLEWYLQKPGQSPKLLIYKVSNRFSGVPDRFSGSGSG
TDFTLKISRVEAEDLGVYYCFQGSHVPYTFGGGTKLEIKRADAAPTV
Mouse anti-CTLA-4 9D9 variable heavy chain nucleic acid sequence (SEQ ID NO: 38)
gaggcaaagcttcaggaatctggtccagtgttggtgaaaccaggtgcatccgtgaaaatgtcctgcaaagcaa
gcggttacacttttactgactattatatgaactgggtaaagcaatcccacggcaaatccctggaatggattgg
tgtcatcaacccttacaacggtgatacaagttacaaccaaaagttcaaaggtaaggctacattgaccgtagat
aagagtagcagtactgcatacatggaacttaactctcttacatccgaggactccgctgtttactattgtgcac
gctactacgggagctggttcgcttactggggtcaaggcaccctgataacagtgtccacagccaaaaccacacc
tccctccgtctatcctctcgctcca
Mouse anti-CTLA-4 9D9 variable heavy chain amino acid sequence (SEQ ID NO: 39)
EAKLQESGPVLVKPGASVKMSCKASGYTFTDYYMNWVKQSHGKSLEWIGVINPYNGDTSYNQKFKGKATLTVD
KSSSTAYMELNSLTSEDSAVYYCARYYGSWFAYWGQGTLITVSTAKTTPPSVYPLAP
Mouse anti-CTLA-4 9H10 variable light chain nucleic acid sequence (SEQ ID NO: 40)
gacattgtgatgacacagagtccttcatcccttgcagtcagtgtcggcgaaaaagtaacaatttcatgcaagt
ctagtcaatctctgttgtacggctcctctcattacctcgcatggtatcaacaaaaagtgggtcaatctcccaa
attgttgatatactgggcttcaactagacacactggaatccctgacaggttcattggtagcggatcagggact
gactttacactgtccctcagcagcgtacaagcagaagacatggccgactatttctgccaacaatactttagta
caccatggacctttggggctgggaccagagttgagataaaa
Mouse anti-CTLA-4 9H10 variable light chain amino acid sequence (SEQ ID NO: 41)
DIVMTQSPSSLAVSVGEKVTISCKSSQSLLYGSSHYLAWYQQKVGQSPKLLIYWASTRHTGIPDRFIGSGSGT
DFTLSLSSVQAEDMADYFCQQYFSTPWTFGAGTRVEIK
Mouse anti-CTLA-4 9H10 variable heavy chain nucleic acid sequence (SEQ ID NO: 42)
caagtgcagctgcttcaatccgaatcagaactcgtgaagccaggcgcttcagtgaaattgtcttgtaagactt
caggatacactttcactgattactatatacactgggttaagcagaagcctggtcagggtcttgaatggattgg
cctcatcaatcccaataacgatggcacaaactacaaccagaaatttcaaggaaaagccacacttaccgcagac
aaatccagttctaccgcatacatggaacttaatagtctcacttttgatgactcagtaatatatttctgtgcca
gggccagtagccgacttagaatggctaggactacctctgactactatgccatggactattggggacagggcat
tcaagtgaccgtgagctct
Mouse anti-CTLA-4 9H10 variable heavy chain amino acid sequence (SEQ ID NO: 43)
QVQLLQSESELVKPGASVKLSCKTSGYTFTDYYIHWVKQKPGQGLEWIGLINPNNDGTNYNQKFQGKATLTAD
KSSSTAYMELNSLTFDDSVIYFCARASSRLRMARTTSDYYAMDYWGQGIQVTVSS
mIgG1 Fc domain nucleic acid sequence (SEQ ID NO: 44)
ggttgtaagccttgcatatgtacagtcccagaagtatcatctgtcttcatcttccccccaaagcccaaggatg
tgctcaccattactctgactcctaaggtcacgtgtgttgtggtagacatcagcaaggatgatcccgaggtcca
gttcagctggtttgtagatgatgtggaggtgcacacagctcagacgcaaccccgggaggagcagttcaacagc
actttccgctcagtcagtgaacttcccatcatgcaccaggactggctcaatggcaaggagttcaaatgcaggg
tcaacagtgcagctttccctgcccccatcgagaaaaccatctccaaaaccaaaggcagaccgaaggctccaca
ggtgtacaccattccacctcccaaggagcagatggccaaggataaagtcagtctgacctgcatgataacagac
ttcttccctgaagacattactgtggagtggcagtggaatgggcagccagcggagaactacaagaacactcagc
ccatcatggacacagatggctcttacttcgtctacagcaagctcaatgtgcagaagagcaactgggaggcagg
aaatactttcacctgctctgtgttacatgagggcctgcacaaccaccatactgagaagagcctctcccactct
cctggtaaatga
mIgG1 Fc domain amino acid sequence (SEQ ID NO: 45)
GCKPCICTVPEVSSVFIFPPKPKDVLTITLTPKVTCVVVDISKDDPEVQFSWFVDDVEVHTAQTQPREEQFNS
TFRSVSELPIMHQDWLNGKEFKCRVNSAAFPAPIEKTISKTKGRPKAPQVYTIPPPKEQMAKDKVSLTCMITD
FFPEDITVEWQWNGQPAENYKNTQPIMDTDGSYFVYSKLNVQKSNWEAGNTFTCSVLHEGLHNHHTEKSLSHS
PGK
OKT3 Variable heavy chain nucleic acid sequence (SEQ ID NO: 46)
caggtgcagctgcagcaatctggggctgaactggcaagacctggggcctcagtgaagatgtcctgcaaggctt
ctggctacacctttactaggtacacgatgcactgggtaaaacagaggcctggacagggtctggaatggattgg
atacattaatcctagccgtggttatactaattacaatcagaagttcaaggacaaggccacattgactacagac
aaatcctccagcacagcctacatgcaactgagcagcctgacatctgaggactctgcagtctattactgtgcaa
gatattatgatgatcattactgccttgactactggggccaaggcaccacactcaccgtctcctca
OKT3 Variable heavy chain amino acid sequence (SEQ ID NO: 47)
QVQLQQSGAELARPGASVKMSCKASGYTFTRYTMHWVKQRPGQGLEWIGYINPSRGYTNYNQKFKDKATLTTD
KSSSTAYMQLSSLTSEDSAVYYCARYYDDHYCLDYWGQGTTLTVSS
OKT3 Variable light chain nucleic acid sequence (SEQ ID NO: 48)
Cagattgtgctcacccagtctccagcaatcatgtctgcatctccaggggagaaggttaccatgacctgcagtg
ccagctcaagtgtaagttacatgaactggtaccagcagaagtcaggcacctcccccaaaagatggatttatga
cacatccaaactggcttctggagtccctgctcacttcaggggcagtgggtctgggacctcttactctctcaca
atcagcggcatggaggctgaagatgctgccacttattactgccagcagtggagtagtaacccattcacgttcg
gctcggggaccaagctggagatcaatcgt
OKT3 Variable light chain nucleic acid sequence (SEQ ID NO: 49)
cagattgtgctcacccagtctccagcaatcatgtctgcatctccaggggagaaggttaccatgacctgcagtg
ccagctcaagtgtaagttacatgaactggtatcagcagaagtcaggcacctcccccaaaagatggatttatga
cacatccaaactggcttctggagtccctgctcacttcaggggcagtgggtctgggacctcttactctctcaca
atcagcggcatggaggctgaagatgctgccacttattactgccagcagtggagtagtaacccattcacgttcg
gctcggggaccaagctggagatcaatcgt
OKT3 Variable light chain amino acid sequence (SEQ ID NO: 50)
QIVLTQSPAIMSASPGEKVTMTCSASSSVSYMNWYQQKSGTSPKRWIYDTSKLASGVPAHFRGSGSGTSYSLT
ISGMEAEDAATYYCQQWSSNPFTFGSGTKLEINR
hIL12-p35 nucleic acid sequence (SEQ ID NO: 51)
tggccccctgggtcagcctcccagccaccgccctcacctgccgcggccacaggtctgcatccagcggctcgcc
ctgtgtccctgcagtgccggctcagcatgtgtccagcgcgcagcctcctccttgtggctaccctggtcctcct
ggaccacctcagtttggccagaaacctccccgtggccactccagacccaggaatgttcccatgccttcaccac
tcccaaaacctgctgagggccgtcagcaacatgctccagaaggccagacaaactctagaattttacccttgca
cttctgaagagattgatcatgaagatatcacaaaagataaaaccagcacagtggaggcctgtttaccattgga
attaaccaagaatgagagttgcctaaattccagagagacctctttcataactaatgggagttgcctggcctcc
agaaagacctcttttatgatggccctgtgccttagtagtatttatgaagacttgaagatgtaccaggtggagt
tcaagaccatgaatgcaaagcttctgatggatcctaagaggcagatctttctagatcaaaacatgctggcagt
tattgatgagctgatgcaggccctgaatttcaacagtgagactgtgccacaaaaatcctcccttgaagaaccg
gatttttataaaactaaaatcaagctctgcatacttcttcatgctttcagaattcgggcagtgactattgata
gagtgatgagctatctgaatgcttcc
hIL12-p35 nucleic acid sequence (SEQ ID NO: 52)
(atg)tggccccctgggtcagcctcccagccaccgccctcacctgccgcggccacaggtctgcatccagcggc
tcgccctgtgtccctgcagtgccggctcagcatgtgtccagcgcgcagcctcctccttgtggctaccctggtc
ctcctggaccacctcagtttggccagaaacctccccgtggccactccagacccaggaatgttcccatgccttc
accactcccaaaacctgctgagggccgtcagcaacatgctccagaaggccagacaaactctcgaattttaccc
ttgcacttctgaagagattgatcatgaagatatcacaaaagataaaaccagcacagtggaggcctgtttacca
ttggaattaaccaagaatgagagttgcctaaattccagagagacctctttcataactaatgggagttgcctgg
cctccagaaagacctcttttatgatggccctgtgccttagtagtatttatgaagacttgaagatgtaccaggt
ggagttcaagaccatgaatgcaaagcttctgatggaccctaagaggcaaatcttcctagatcaaaacatgctg
gcagttattgatgagctgatgcaggccctgaatttcaacagtgagactgtgccacaaaaatcctcccttgaag
aaccggatttctacaagactaaaatcaagctctgcatacttcttcatgctttcagaatccgggcagtgactat
tgatagagtgatgagctatctgaatgcttcc
hIL12-p35 amino acid sequence (SEQ ID NO: 53)
(M)WPPGSASQPPPSPAAATGLHPAARPVSLQCRLSMCPARSLLLVATLVLLDHLSLARNLPVATPDPGMFPC
LHHSQNLLRAVSNMLQKARQTLEFYPCTSEEIDHEDITKDKTSTVEACLPLELTKNESCLNSRETSFITNGSC
LASRKTSFMMALCLSSIYEDLKMYQVEFKTMNAKLLMDPKRQIFLDQNMLAVIDELMQALNFNSETVPQKSSL
EEPDFYKTKIKLCILLHAFRIRAVTIDRVMSYLNAS
hIL12-p40 nucleic acid sequence (SEQ ID NO: 54)
Tgtcaccagcagttggtcatctcttggttttccctggtttttctggcatctcccctcgtggccatatgggaac
tgaagaaagatgtttatgtcgtagaattggattggtatccggatgcccctggagaaatggtggtcctcacctg
tgacacccctgaagaagatggtatcacctggaccttggaccagagcagtgaggtcttaggctctggcaaaacc
ctgaccatccaagtcaaagagtttggagatgctggccagtacacctgtcacaaaggaggcgaggttctaagcc
attcgctcctgctgcttcacaaaaaggaagatggaatttggtccactgatattttaaaggaccagaaagaacc
caaaaataagacctttctaagatgcgaggccaagaattattctggacgtttcacctgctggtggctgacgaca
atcagtactgatttgacattcagtgtcaaaagcagcagaggctcttctgacccccaaggggtgacgtgcggag
ctgctacactctctgcagagagagtcagaggggacaacaaggagtatgagtactcagtggagtgccaggagga
cagtgcctgcccagctgctgaggagagtctgcccattgaggtcatggtggatgccgttcacaagctcaagtat
gaaaactacaccagcagcttcttcatcagggacatcatcaaacctgacccacccaagaacttgcagctgaagc
cattaaagaattctcggcaggtggaggtcagctgggagtaccctgacacctggagtactccacattcctactt
ctccctgacattctgcgttcaggtccagggcaagagcaagagagaaaagaaagatagagtcttcacggacaag
acctcagccacggtcatctgccgcaaaaatgccagcattagcgtgcgggcccaggaccgctactatagctcat
cttggagcgaatgggcatctgtgccctgcagt(tag)
hIL12-p40 nucleic acid sequence (SEQ ID NO: 55)
tgtcaccagcagttggtcatctcttggttttccctggtttttctggcatctcccctcgtggccatatgggaac
tgaagaaagatgtttatgtcgtagaattggattggtatccggatgcccctggagaaatggtggtcctcacctg
tgacacccctgaagaagatggtatcacctggaccttggaccagagcagtgaggtcttaggctctggcaaaacc
ctgaccatccaagtcaaagagtttggagatgctggccagtacacctgtcacaaaggaggcgaggttctaagcc
attcgctcctgctgcttcacaaaaaggaagatggaatttggtccactgatattttaaaggaccagaaagaacc
caaaaataagacctttctaagatgcgaggccaagaattattctggacgtttcacctgctggtggctgacgaca
atcagtactgatttgacattcagtgtcaaaagcagcagaggctcttctgacccccaaggggtgacgtgcggag
ctgctacactctctgcagagagagtcagaggggacaacaaggagtatgagtactcagtggagtgccaggagga
cagtgcctgcccagctgctgaggagagtctgcccattgaggtcatggtggatgccgttcacaagctcaagtat
gaaaactacaccagcagcttcttcatcagggacatcatcaaacctgacccacccaagaacttgcagctgaagc
cattaaagaactctcggcaggtggaggtcagctgggagtaccctgacacctggagtactccacattcctactt
ctccctgacattctgcgttcaggtccagggcaagagcaagagagaaaagaaagatagagtcttcacggacaag
acctcagccacggtcatctgccgcaaaaatgccagcattagcgtgcgggcccaggaccgctactatagctcat
cttggagcgaatgggcatctgtgccctgcagttcg
hIL-12p40 amino acid sequence (SEQ ID NO: 56)
CHQQLVISWFSLVFLASPLVAIWELKKDVYVVELDWYPDAPGEMVVLTCDTPEEDGITWTLDQSSEVLGSGKT
LTIQVKEFGDAGQYTCHKGGEVLSHSLLLLHKKEDGIWSTDILKDQKEPKNKTFLRCEAKNYSGRFTCWWLTT
ISTDLTFSVKSSRGSSDPQGVTCGAATLSAERVRGDNKEYEYSVECQEDSACPAAEESLPIEVMVDAVHKLKY
ENYTSSFFIRDIIKPDPPKNLQLKPLKNSRQVEVSWEYPDTWSTPHSYFSLTFCVQVQGKSKREKKDRVFTDK
TSATVICRKNASISVRAQDRYYSSSWSEWASVPCS
Human CXCL9 (hCXCL9) nucleic acid sequence (SEQ ID NO: 57)
(atg)aagaaaagtggtgttcttttcctcttgggcatcatcttgctggttctgattggagtgcaaggaacccc
agtagtgagaaagggtcgctgttcctgcatcagcaccaaccaagggactatccacctacaatccttgaaagac
cttaaacaatttgccccaagcccttcctgcgagaaaattgaaatcattgctacactgaagaatggagttcaaa
catgtctaaacccagattcagcagatgtgaaggaactgattaaaaagtgggagaaacaggtcagccaaaagaa
aaagcaaaagaatgggaaaaaacatcaaaaaaagaaagttctgaaagttcgaaaatctcaacgttctcgtcaa
aagaagactacataa
Human CXCL9 (hCXCL9) amino acid sequence (SEQ ID NO: 58)
(M)KKSGVLFLLGIILLVLIGVQGTPVVRKGRCSCISTNQGTIHLQSLKDLKQFAPSPSCEKIEIIATLKNGV
QTCLNPDSADVKELIKKWEKQVSQKKKQKNGKKHQKKKVLKVRKSQRSRQKKTT
Igκ-HA-2C11VHC-Linker-C211VLC-Myc-PDGFR nucleic acid sequence (SEQ ID NO: 59)
Atggagacagacacactcctgctatgggtactgctgctctgggttccaggttccactggtgactatccatatg
atgttccagattatgctggggcccagccggccagatctgaggtgcagctggtggagtctgggggaggcttggt
gcagcctggaaagtccctgaaactctcctgtgaggcctctggattcaccttcagcggctatggcatgcactgg
gtccgccaggctccagggagggggctggagtcggtcgcatacattactagtagtagtattaatatcaaatatg
ctgacgctgtgaaaggccggttcaccgtctccagagacaatgccaagaacttactgtttctacaaatgaacat
tctcaagtctgaggacacagccatgtactactgtgcaagattcgactgggacaaaaattactggggccaagga
accatggtcaccgtctcctcaggtggcggtggctccggcggtggtgggtcgggtggcggcggatctgacatcc
agatgacccagtctccatcatcactgcctgcctccctgggagacagagtcactatcaattgtcaggccagtca
ggacattagcaattatttaaactggtaccagcagaaaccagggaaagctcctaagctcctgatctattataca
aataaattggcagatggagtcccatcaaggttcagtggcagtggttctgggagagattcttctttcactatca
gcagcctggaatccgaagatattggatcttattactgtcaacagtattataactatccgtggacgttcggacc
tggcaccaagctggaaatcaaagtcgacgaacaaaaactcatctcagaagaggatctgtacactgtgggccag
gacacgcaggaggtcatcgtggtgccacactccttgccctttaaggtggtggtgatctcagccatcctggccc
tggtggtgctcaccatcatctcccttatcatcctcatcatgctttggcagaagaagccacgt(tag
Igκ-HA-2C11VHC-Linker-C211VLC-Myc-PDGFR amino acid sequence (SEQ ID NO: 60)
METDTLLLWVLLLWVPGSTGDYPYDVPDYAGAQPARSEVQLVESGGGLVQPGKSLKLSCEASGFTFSGYGMHW
VRQAPGRGLESVAYITSSSINIKYADAVKGRFTVSRDNAKNLLFLQMNILKSEDTAMYYCARFDWDKNYWGQG
TMVTVSSGGGGSGGGGSGGGGSDIQMTQSPSSLPASLGDRVTINCQASQDISNYLNWYQQKPGKAPKLLIYYT
NKLADGVPSRFSGSGSGRDSSFTISSLESEDIGSYYCQQYYNYPWTFGPGTKLEIKVDEQKLISEEDLYTVGQ
DTQEVIVVPHSLPFKVVVISAILALVVLTIISLIILIMLWQKKPR
IgK-HA-2C11VHC-Linker-2C11VLC-Linker-PDGFR nucleic acid sequence (SEQ ID NO: 61)
Atggagacagacacactcctgctatgggtactgctgctctgggttccaggttccactggtgactatccatatg
atgttccagattatgctggggcccagccggccagatctgaggtgcagctggtggagtctgggggaggcttggt
gcagcctggaaagtccctgaaactctcctgtgaggcctctggattcaccttcagcggctatggcatgcactgg
gtccgccaggctccagggagggggctggagtcggtcgcatacattactagtagtagtattaatatcaaatatg
ctgacgctgtgaaaggccggttcaccgtctccagagacaatgccaagaacttactgtttctacaaatgaacat
tctcaagtctgaggacacagccatgtactactgtgcaagattcgactgggacaaaaattactggggccaagga
accatggtcaccgtctcctcaggtggcggtggctccggcggtggtgggtcgggtggcggcggatctgacatcc
agatgacccagtctccatcatcactgcctgcctccctgggagacagagtcactatcaattgtcaggccagtca
ggacattagcaattatttaaactggtatcagcagaaaccagggaaagctcctaagctcctgatctattataca
aataaattggcagatggagtcccatcaaggttcagtggcagtggttctgggagagattcttctttcactatca
gcagcctggaatccgaagatattggatcttattactgtcaacagtattataactatccgtggacgttcggacc
tggcaccaagctggaaatcaaaggcagtgggagtgggagtgggagtgggaatgctgtgggccaggacacgcag
gaggtcatcgtggtgccacactccttgccctttaaggtggtggtgatctcagccatcctggccctggtggtgc
tcaccatcatctcccttatcatcctcatcatgctttggcagaagaagccacgt(TAG)
IgK-HA-2C11VHC-Linker-2C11VLC-Linker-PDGFR amino acid sequence (SEQ ID NO: 62)
METDTLLLWVLLLWVPGSTGDYPYDVPDYAGAQPARSEVQLVESGGGLVQPGKSLKLSCEASGFTFSGYGMHW
VRQAPGRGLESVAYITSSSINIKYADAVKGRFTVSRDNAKNLLFLQMNILKSEDTAMYYCARFDWDKNYWGQG
TMVTVSSGGGGSGGGGSGGGGSDIQMTQSPSSLPASLGDRVTINCQASQDISNYLNWYQQKPGKAPKLLIYYT
NKLADGVPSRFSGSGSGRDSSFTISSLESEDIGSYYCQQYYNYPWTFGPGTKLEIKGSGSGSGSGNAVGQDTQ
EVIVVPHSLPFKVVVISAILALVVLTIISLIILIMLWQKKPR
IgK-HA-2C11VHC-Linker-2C11VLC-Linker-PDGFR-P2A-mIL12-p35-P2A-mIL12-p40 nucleic
acid sequence (SEQ ID NO: 63)
Atggagacagacacactcctgctatgggtactgctgctctgggttccaggttccactggtgactatccatatg
atgttccagattatgctggggcccagccggccagatctgaggtgcagctggtggagtctgggggaggcttggt
gcagcctggaaagtccctgaaactctcctgtgaggcctctggattcaccttcagcggctatggcatgcactgg
gtccgccaggctccagggagggggctggagtcggtcgcatacattactagtagtagtattaatatcaaatatg
ctgacgctgtgaaaggccggttcaccgtctccagagacaatgccaagaacttactgtttctacaaatgaacat
tctcaagtctgaggacacagccatgtactactgtgcaagattcgactgggacaaaaattactggggccaagga
accatggtcaccgtctcctcaggtggcggtggctccggcggtggtgggtcgggtggcggcggatctgacatcc
agatgacccagtctccatcatcactgcctgcctccctgggagacagagtcactatcaattgtcaggccagtca
ggacattagcaattatttaaactggtatcagcagaaaccagggaaagctcctaagctcctgatctattataca
aataaattggcagatggagtcccatcaaggttcagtggcagtggttctgggagagattcttctttcactatca
gcagcctggaatccgaagatattggatcttattactgtcaacagtattataactatccgtggacgttcggacc
tggcaccaagctggaaatcaaaggcagtgggagtgggaatgctgtgggccaggacacgcaggaggtcatcgtg
gtgccacactccttgccctttaaggtggtggtgatctcagccatcctggccctggtggtgctcaccatcatct
cccttatcatcctcatcatgctttggcagaagaagccacgtggatctggggccaccaacttttcattgctcaa
gcaggcgggcgatgtggaggaaaaccctggccccggtaccgtcagcgttccaacagcctcaccctcggcatcc
agcagctcctctcagtgccggtccagcatgtgtcaatcacgctacctcctctttttggccacccttgccctcc
taaaccacctcagtttggccagggtcattccagtctctggacctgccaggtgtcttagccagtcccgaaacct
gctgaagaccacagatgacatggtgaagacggccagagaaaaactgaaacattattcctgcactgctgaagac
atcgatcatgaagacatcacacgggaccaaaccagcacattgaagacctgtttaccactggaactacacaaga
acgagagttgcctggctactagagagacttcttccacaacaagagggagctgcctgcccccacagaagacgtc
tttgatgatgaccctgtgccttggtagcatctatgaggacttgaagatgtaccagacagagttccaggccatc
aacgcagcacttcagaatcacaaccatcagcagatcattcttgacaagggcatgctggtggccatcgatgagc
tgatgcagtctctgaatcataatggcgagactctgcgccagaaacctcctgtgggagaagcagacccttacag
agtgaaaatgaagctctgcatcctgcttcacgccttcagcacccgcgtcgtgaccatcaacagggtgatgggc
tatctgagctccgccgcggccgcaggatctggggccaccaacttttcattgctcaagcaggcgggcgatgtgg
aggaaaaccctggccccggatcctgtcctcagaagctaaccatctcctggtttgccatcgttttgctggtgtc
tccactcatggccatgtgggagctggagaaagacgtttatgttgtagaggtggactggactcccgatgcccct
ggagaaacagtgaacctcacctgtgacacgcctgaagaagatgacatcacctggacctcagaccagagacatg
gagtcataggctctggaaagaccctgaccatcactgtcaaagagtttcttgatgctggccagtacacctgcca
caaaggaggcgagactctgagccactcacatctgctgctccacaagaaggaaaatggaatttggtccactgaa
attttaaagaatttcaagaacaagactttcctgaagtgtgaagcaccaaattactccggacggttcacgtgct
catggctggtgcaaagaaacatggacttgaagttcaacatcaagagcagtagcagttcccctgactctcgggc
agtgacatgtggaatggcgtctctgtctgcagagaaggtcacactggaccaaagggactatgagaagtattca
gtgtcctgccaggaggatgtcacctgcccaactgccgaggagaccctgcccattgaactggcgttggaagcac
ggcagcagaataaatatgagaactacagcaccagcttcttcatcagggacatcatcaaaccagacccgcccaa
gaacttgcagatgaagcctttgaagaactcacaggtggaggtcagctgggagtaccctgactcctggagcact
ccccattcctacttctccctcaagttctttgttcgaatccagcgcaagaaagaaaagatgaaggagacagagg
aggggtgtaaccagaaaggtgcgttcctcgtagagaagacatctaccgaagtccaatgcaaaggcgggaatgt
ctgcgtgcaagctcaggatcgctattacaattcctcatgcagcaagtgggcatgtgttccctgcagggtccga
tcctag
Igκ-HA-2C11VHC-Linker-2C11VLC-Linker-PDGFR-P2A-mIL12-p35-P2A-mIL12-p40 amino
acid sequence (SEQ ID NO: 64)
METDTLLLWVLLLWVPGSTGDYPYDVPDYAGAQPARSEVQLVESGGGLVQPGKSLKLSCEASGFTFSGYGMHW
VRQAPGRGLESVAYITSSSINIKYADAVKGRFTVSRDNAKNLLFLQMNILKSEDTAMYYCARFDWDKNYWGQG
TMVTVSSGGGGSGGGGSGGGGSDIQMTQSPSSLPASLGDRVTINCQASQDISNYLNWYQQKPGKAPKLLIYYT
NKLADGVPSRFSGSGSGRDSSFTISSLESEDIGSYYCQQYYNYPWTFGPGTKLEIKGSGSGNAVGQDTQEVIV
VPHSLPFKVVVISAILALVVLTIISLIILIMLWQKKPRGSGATNFSLLKQAGDVEENPGPGTVSVPTASPSAS
SSSSQCRSSMCQSRYLLFLATLALLNHLSLARVIPVSGPARCLSQSRNLLKTTDDMVKTAREKLKHYSCTAED
IDHEDITRDQTSTLKTCLPLELHKNESCLATRETSSTTRGSCLPPQKTSLMMTLCLGSIYEDLKMYQTEFQAI
NAALQNHNHQQIILDKGMLVAIDELMQSLNHNGETLRQKPPVGEADPYRVKMKLCILLHAFSTRVVTINRVMG
YLSSAAAAGSGATNFSLLKQAGDVEENPGPGSCPQKLTISWFAIVLLVSPLMAMWELEKDVYVVEVDWTPDAP
GETVNLTCDTPEEDDITWTSDQRHGVIGSGKTLTITVKEFLDAGQYTCHKGGETLSHSHLLLHKKENGIWSTE
ILKNFKNKTFLKCEAPNYSGRFTCSWLVQRNMDLKFNIKSSSSSPDSRAVTCGMASLSAEKVTLDQRDYEKYS
VSCQEDVTCPTAEETLPIELALEARQQNKYENYSTSFFIRDIIKPDPPKNLQMKPLKNSQVEVSWEYPDSWST
PHSYFSLKFFVRIQRKKEKMKETEEGCNQKGAFLVEKTSTEVQCKGGNVCVQAQDRYYNSSCSKWACVPCRVR
S
Igκ-2C11VHC-Linker-2C11VLC-Linker-PDGFR-P2A-mIL12-p35-P2A-mIL12-p40 nucleic
acid sequence (SEQ ID NO: 65)
atggagacagacacactcctgctatgggtactgctgctctgggttccaggttccactggtgacggggcccagc
cggccagatctgaggtgcagctggtggagtctgggggaggcttggtgcagcctggaaagtccctgaaactctc
ctgtgaggcctctggattcaccttcagcggctatggcatgcactgggtccgccaggctccagggagggggctg
gagtcggtcgcatacattactagtagtagtattaatatcaaatatgctgacgctgtgaaaggccggttcaccg
tctccagagacaatgccaagaacttactgtttctacaaatgaacattctcaagtctgaggacacagccatgta
ctactgtgcaagattcgactgggacaaaaattactggggccaaggaaccatggtcaccgtctcctcaggtggc
ggtggctccggcggtggtgggtcgggtggcggcggatctgacatccagatgacccagtctccatcatcactgc
ctgcctccctgggagacagagtcactatcaattgtcaggccagtcaggacattagcaattatttaaactggta
tcagcagaaaccagggaaagctcctaagctcctgatctattatacaaataaattggcagatggagtcccatca
aggttcagtggcagtggttctgggagagattcttctttcactatcagcagcctggaatccgaagatattggat
cttattactgtcaacagtattataactatccgtggacgttcggacctggcaccaagctggaaatcaaaggcag
tgggagtgggaatgctgtgggccaggacacgcaggaggtcatcgtggtgccacactccttgccctttaaggtg
gtggtgatctcagccatcctggccctggtggtgctcaccatcatctcccttatcatcctcatcatgctttggc
agaagaagccacgtggatctggggccaccaacttttcattgctcaagcaggcgggcgatgtggaggaaaaccc
tggccccggtaccgtcagcgttccaacagcctcaccctcggcatccagcagctcctctcagtgccggtccagc
atgtgtcaatcacgctacctcctctttttggccacccttgccctcctaaaccacctcagtttggccagggtca
ttccagtctctggacctgccaggtgtcttagccagtcccgaaacctgctgaagaccacagatgacatggtgaa
gacggccagagaaaaactgaaacattattcctgcactgctgaagacatcgatcatgaagacatcacacgggac
caaaccagcacattgaagacctgtttaccactggaactacacaagaacgagagttgcctggctactagagaga
cttcttccacaacaagagggagctgcctgcccccacagaagacgtctttgatgatgaccctgtgccttggtag
catctatgaggacttgaagatgtaccagacagagttccaggccatcaacgcagcacttcagaatcacaaccat
cagcagatcattcttgacaagggcatgctggtggccatcgatgagctgatgcagtctctgaatcataatggcg
agactctgcgccagaaacctcctgtgggagaagcagacccttacagagtgaaaatgaagctctgcatcctgct
tcacgccttcagcacccgcgtcgtgaccatcaacagggtgatgggctatctgagctccgccgcggccgcagga
tctggggccaccaacttttcattgctcaagcaggcgggcgatgtggaggaaaaccctggccccggatcctgtc
ctcagaagctaaccatctcctggtttgccatcgttttgctggtgtctccactcatggccatgtgggagctgga
gaaagacgtttatgttgtagaggtggactggactcccgatgcccctggagaaacagtgaacctcacctgtgac
acgcctgaagaagatgacatcacctggacctcagaccagagacatggagtcataggctctggaaagaccctga
ccatcactgtcaaagagtttcttgatgctggccagtacacctgccacaaaggaggcgagactctgagccactc
acatctgctgctccacaagaaggaaaatggaatttggtccactgaaattttaaagaatttcaagaacaagact
ttcctgaagtgtgaagcaccaaattactccggacggttcacgtgctcatggctggtgcaaagaaacatggact
tgaagttcaacatcaagagcagtagcagttcccctgactctcgggcagtgacatgtggaatggcgtctctgtc
tgcagagaaggtcacactggaccaaagggactatgagaagtattcagtgtcctgccaggaggatgtcacctgc
ccaactgccgaggagaccctgcccattgaactggcgttggaagcacggcagcagaataaatatgagaactaca
gcaccagcttcttcatcagggacatcatcaaaccagacccgcccaagaacttgcagatgaagcctttgaagaa
ctcacaggtggaggtcagctgggagtaccctgactcctggagcactccccattcctacttctccctcaagttc
tttgttcgaatccagcgcaagaaagaaaagatgaaggagacagaggaggggtgtaaccagaaaggtgcgttcc
tcgtagagaagacatctaccgaagtccaatgcaaaggcgggaatgtctgcgtgcaagctcaggatcgctatta
caattcctcatgcagcaagtgggcatgtgttccctgcagggtccgatcctag
Igκ-2C11VHC-Linker-2C11VLC-Linker-PDGFR-P2A-mIL12-p35-P2A-mIL12-p40 amino
acid sequence (SEQ ID NO: 66)
METDTLLLWVLLLWVPGSTGDGAQPARSEVQLVESGGGLVQPGKSLKLSCEASGFTFSGYGMHWVRQAPGRGL
ESVAYITSSSINIKYADAVKGRFTVSRDNAKNLLFLQMNILKSEDTAMYYCARFDWDKNYWGQGTMVTVSSGG
GGSGGGGSGGGGSDIQMTQSPSSLPASLGDRVTINCQASQDISNYLNWYQQKPGKAPKLLIYYTNKLADGVPS
RFSGSGSGRDSSFTISSLESEDIGSYYCQQYYNYPWTFGPGTKLEIKGSGSGNAVGQDTQEVIVVPHSLPFKV
VVISAILALVVLTIISLIILIMLWQKKPRGSGATNFSLLKQAGDVEENPGPGTVSVPTASPSASSSSSQCRSS
MCQSRYLLFLATLALLNHLSLARVIPVSGPARCLSQSRNLLKTTDDMVKTAREKLKHYSCTAEDIDHEDITRD
QTSTLKTCLPLELHKNESCLATRETSSTTRGSCLPPQKTSLMMTLCLGSIYEDLKMYQTEFQAINAALQNHNH
QQIILDKGMLVAIDELMQSLNHNGETLRQKPPVGEADPYRVKMKLCILLHAFSTRVVTINRVMGYLSSAAAAG
SGATNFSLLKQAGDVEENPGPGSCPQKLTISWFAIVLLVSPLMAMWELEKDVYVVEVDWTPDAPGETVNLTCD
TPEEDDITWTSDQRHGVIGSGKTLTITVKEFLDAGQYTCHKGGETLSHSHLLLHKKENGIWSTEILKNFKNKT
FLKCEAPNYSGRFTCSWLVQRNMDLKFNIKSSSSSPDSRAVTCGMASLSAEKVTLDQRDYEKYSVSCQEDVTC
PTAEETLPIELALEARQQNKYENYSTSFFIRDIIKPDPPKNLQMKPLKNSQVEVSWEYPDSWSTPHSYFSLKF
FVRIQRKKEKMKETEEGCNQKGAFLVEKTSTEVQCKGGNVCVQAQDRYYNSSCSKWACVPCRVRS
mIL12-p35-P2A-mIL12-p40-P2A-mCXCL9 nucleic acid sequence (SEQ ID NO: 67)
atggtcagcgttccaacagcctcaccctcggcatccagcagctcctctcagtgccggtccagcatgtgtcaat
cacgctacctcctctttttggccacccttgccctcctaaaccacctcagtttggccagggtcattccagtctc
tggacctgccaggtgtcttagccagtcccgaaacctgctgaagaccacagatgacatggtgaagacggccaga
gaaaaactgaaacattattcctgcactgctgaagacatcgatcatgaagacatcacacgggaccaaaccagca
cattgaagacctgtttaccactggaactacacaagaacgagagttgcctggctactagagagacttcttccac
aacaagagggagctgcctgcccccacagaagacgtctttgatgatgaccctgtgccttggtagcatctatgag
gacttgaagatgtaccagacagagttccaggccatcaacgcagcacttcagaatcacaaccatcagcagatca
ttctTgacaagggcatgctggtggccatcgatgagctgatgcagtctctgaatcataatggcgagactctgcg
ccagaaacctcctgtgggagaagcagacccttacagagtgaaaatgaagctctgcatcctgcttcacgccttc
agcacccgcgtcgtgaccatcaacagggtgatgggctatctgagctccgccGCGGCCGCAggatctggggcca
ccaacttttcattgctcaagcaggcgggcgatgtggaggaaaaccctggccccGGATCCtgtcctcagaagct
aaccatctcctggtttgccatcgttttgctggtgtctccactcatggccatgtgggagctggagaaagacgtt
tatgttgtagaggtggactggactcccgatgcccctggagaaacagtgaacctcacctgtgacacgcctgaag
aagatgacatcacctggacctcagaccagagacatggagtcataggctctggaaagaccctgaccatcactgt
caaagagtttcttgatgctggccagtacacctgccacaaaggaggcgagactctgagccactcacatctgctg
ctccacaagaaggaaaatggaatttggtccactgaaattttaaaGaatttcaaGaacaagactttcctgaagt
gtgaagcaccaaattactccggacggttcacgtgctcatggctggtgcaaagaaacatggacttgaagttcaa
catcaagagcagtagcagttcccctgactctcgggcagtgacatgtggaatggcgtctctgtctgcagagaag
gtcacactggaccaaagggactatgagaagtattcagtgtcctgccaggaggatgtcacctgcccaactgccg
aggagaccctgcccattgaactggcgttggaagcacggcagcagaataaatatgagaactacagcaccagctt
cttcatcagggacatcatcaaaccagacccgcccaagaacttgcagatgaagcctttgaagaactcacaggtg
gaggtcagctgggagtaccctgactcctggagcactccccattcctacttctccctcaagttctttgttcgaa
tccagcgcaagaaagaaaagatgaaggagacagaggaggggtgtaaccagaaaggtgcgttcctcgtagagaa
gacatctaccgaagtccaatgcaaaggcgggaatgtctgcgtgcaagctcaggatcgctattacaattcctca
tgcagcaagtgggcatgtgttccctgcagggtccgatcctCgTCTAGAggatctggggccaccaacttttcat
tgctcaagcaggcgggcgatgtggaggaaaaccctggccccaagtccgctgttcttttcctcttgggcatcat
cttcctggagcagtgtggagttcgaggaaccctagtgataaggaatgcacgatgctcctgcatcagcaccagc
cgaggcacgatccactacaaatccctcaaagacctcaaacagtttgccccaagccccaattgcaacaaaactg
aaatcattgctacactgaagaacggagatcaaacctgcctagatccggactcggcaaatgtgaagaagctgat
gaaagaatgggaaaagaagatcagccaaaagaaaaagcaaaagagggggaaaaaacatcaaaagaacatgaaa
aacagaaaacccaaaacaccccaaagtcgtcgtcgttcaaggaagactacataa
mIL12-p35-P2A-mIL12-p40-P2A-mCXCL9 amino acid sequence (SEQ ID NO: 68)
MVSVPTASPSASSSSSQCRSSMCQSRYLLFLATLALLNHLSLARVIPVSGPARCLSQSRNLLKTTDDMVKTAR
EKLKHYSCTAEDIDHEDITRDQTSTLKTCLPLELHKNESCLATRETSSTTRGSCLPPQKTSLMMTLCLGSIYE
DLKMYQTEFQAINAALQNHNHQQIILDKGMLVAIDELMQSLNHNGETLRQKPPVGEADPYRVKMKLCILLHAF
STRVVTINRVMGYLSSAAAAGSGATNFSLLKQAGDVEENPGPGSCPQKLTISWFAIVLLVSPLMAMWELEKDV
YVVEVDWTPDAPGETVNLTCDTPEEDDITWTSDQRHGVIGSGKTLTITVKEFLDAGQYTCHKGGETLSHSHLL
LHKKENGIWSTEILKNFKNKTFLKCEAPNYSGRFTCSWLVQRNMDLKFNIKSSSSSPDSRAVTCGMASLSAEK
VTLDQRDYEKYSVSCQEDVTCPTAEETLPIELALEARQQNKYENYSTSFFIRDIIKPDPPKNLQMKPLKNSQV
EVSWEYPDSWSTPHSYFSLKFFVRIQRKKEKMKETEEGCNQKGAFLVEKTSTEVQCKGGNVCVQAQDRYYNSS
CSKWACVPCRVRSSSRGSGATNFSLLKQAGDVEENPGPKSAVLFLLGIIFLEQCGVRGTLVIRNARCSCISTS
RGTIHYKSLKDLKQFAPSPNCNKTEIIATLKNGDQTCLDPDSANVKKLMKEWEKKISQKKKQKRGKKHQKNMK
NRKPKTPQSRRRSRKTT
Igκ-HA-9D9 VLC-Linker-9D9 VHC-Linker-mIgG1 Fc domain (anti-CTLA-4 scFv)
nucleic acid sequence (SEQ ID NO: 69)
atggagacagacacactcctgctatgggtactgctgctctgggttccaggttccactggtgactatccatatg
atgttccagattatgctggggcccagccggccagatctgacattgtgatgacacagaccacactcagtctccc
cgtttcccttggtgatcaagcctccatatcctgtaggtctagtcaatctatcgtccactccaacggcaatacc
tatctggaatggtatcttcaaaagcccggacaatcaccaaagcttcttatctataaggtgagcaatagattta
gcggggtccctgaccgattctctggaagtggctctggcacagactttaccttgaaaatctccagagttgaggc
tgaggaccttggtgtatactactgcttccaaggctctcatgttccctacactttcggaggcggaacaaaactg
gagataaaacgagccgacgcagcccccactgtgagtggctctggagggggctctggcggtggatctgggggtg
gaagtgaggcaaagcttcaggaatctggtccagtgttggtgaaaccaggtgcatccgtgaaaatgtcctgcaa
agcaagcggttacacttttactgactattatatgaactgggtaaagcaatcccacggcaaatccctggaatgg
attggtgtcatcaacccttacaacggtgatacaagttacaaccaaaagttcaaaggtaaggctacattgaccg
tagataagagtagcagtactgcatacatggaacttaactctcttacatccgaggactccgctgtttactattg
tgcacgctactacgggagctggttcgcttactggggtcaaggcaccctgataacagtgtccacagccaaaacc
acacctccctccgtctatcctctcgctccagtcgactctagtggatccggtggttgtaagccttgcatatgta
cagtcccagaagtatcatctgtcttcatcttccccccaaagcccaaggatgtgctcaccattactctgactcc
taaggtcacgtgtgttgtggtagacatcagcaaggatgatcccgaggtccagttcagctggtttgtagatgat
gtggaggtgcacacagctcagacgcaaccccgggaggagcagttcaacagcactttccgctcagtcagtgaac
ttcccatcatgcaccaggactggctcaatggcaaggagttcaaatgcagggtcaacagtgcagctttccctgc
ccccatcgagaaaaccatctccaaaaccaaaggcagaccgaaggctccacaggtgtacaccattccacctccc
aaggagcagatggccaaggataaagtcagtctgacctgcatgataacagacttcttccctgaagacattactg
tggagtggcagtggaatgggcagccagcggagaactacaagaacactcagcccatcatggacacagatggctc
ttacttcgtctacagcaagctcaatgtgcagaagagcaactgggaggcaggaaatactttcacctgctctgtg
ttacatgagggcctgcacaaccaccatactgagaagagcctctcccactctcctggtaaatga
Igκ-HA-9D9 VLC-Linker-9D9 VHC-Linker-mIgG1 Fc domain (anti-CTLA-4 scFv)
amino acid sequence (SEQ ID NO: 70)
METDTLLLWVLLLWVPGSTGDYPYDVPDYAGAQPARSDIVMTQTTLSLPVSLGDQASISCRSSQSIVHSNGNT
YLEWYLQKPGQSPKLLIYKVSNRFSGVPDRFSGSGSGTDFTLKISRVEAEDLGVYYCFQGSHVPYTFGGGTKL
EIKRADAAPTVSGSGGGSGGGSGGGSEAKLQESGPVLVKPGASVKMSCKASGYTFTDYYMNWVKQSHGKSLEW
IGVINPYNGDTSYNQKFKGKATLTVDKSSSTAYMELNSLTSEDSAVYYCARYYGSWFAYWGQGTLITVSTAKT
TPPSVYPLAPVDSSGSGGCKPCICTVPEVSSVFIFPPKPKDVLTITLTPKVTCVVVDISKDDPEVQFSWFVDD
VEVHTAQTQPREEQFNSTFRSVSELPIMHQDWLNGKEFKCRVNSAAFPAPIEKTISKTKGRPKAPQVYTIPPP
KEQMAKDKVSLTCMITDFFPEDITVEWQWNGQPAENYKNTQPIMDTDGSYFVYSKLNVQKSNWEAGNTFTCSV
LHEGLHNHHTEKSLSHSPGK
Igκ-HA-9H10 VLC-Linker-9H10 VHC-Linker-mIgG1 Fc domain (anti-CTLA-4 scFv)
nucleic acid sequence (SEQ ID NO: 71)
atggagacagacacactcctgctatgggtactgctgctctgggttccaggttccactggtgactatccatatg
atgttccagattatgctggggcccagccggccagatctgacattgtgatgacacagagtccttcatcccttgc
agtcagtgtcggcgaaaaagtaacaatttcatgcaagtctagtcaatctctgttgtacggctcctctcattac
ctcgcatggtatcaacaaaaagtgggtcaatctcccaaattgttgatatactgggcttcaactagacacactg
gaatccctgacaggttcattggtagcggatcagggactgactttacactgtccctcagcagcgtacaagcaga
agacatggccgactatttctgccaacaatactttagtacaccatggacctttggggctgggaccagagttgag
ataaaaagtggctctggagggggctctggcggtggatctgggggtggaagtcaagtgcagctgcttcaatccg
aatcagaactcgtgaagccaggcgcttcagtgaaattgtcttgtaagacttcaggatacactttcactgatta
ctatatacactgggttaagcagaagcctggtcagggtcttgaatggattggcctcatcaatcccaataacgat
ggcacaaactacaaccagaaatttcaaggaaaagccacacttaccgcagacaaatccagttctaccgcataca
tggaacttaatagtctcacttttgatgactcagtaatatatttctgtgccagggccagtagccgacttagaat
ggctaggactacctctgactactatgccatggactattggggacagggcattcaagtgaccgtgagctctgtc
gactctagtggatccggtggttgtaagccttgcatatgtacagtcccagaagtatcatctgtcttcatcttcc
ccccaaagcccaaggatgtgctcaccattactctgactcctaaggtcacgtgtgttgtggtagacatcagcaa
ggatgatcccgaggtccagttcagctggtttgtagatgatgtggaggtgcacacagctcagacgcaaccccgg
gaggagcagttcaacagcactttccgctcagtcagtgaacttcccatcatgcaccaggactggctcaatggca
aggagttcaaatgcagggtcaacagtgcagctttccctgcccccatcgagaaaaccatctccaaaaccaaagg
cagaccgaaggctccacaggtgtacaccattccacctcccaaggagcagatggccaaggataaagtcagtctg
acctgcatgataacagacttcttccctgaagacattactgtggagtggcagtggaatgggcagccagcggaga
actacaagaacactcagcccatcatggacacagatggctcttacttcgtctacagcaagctcaatgtgcagaa
gagcaactgggaggcaggaaatactttcacctgctctgtgttacatgagggcctgcacaaccaccatactgag
aagagcctctcccactctcctggtaaatga
Igκ-HA-9H10 VLC-Linker-9H10 VHC-Linker-mIgG1 Fc domain (anti-CTLA-4 scFv)
amino acid sequence (SEQ ID NO: 72)
METDTLLLWVLLLWVPGSTGDYPYDVPDYAGAQPARSDIVMTQSPSSLAVSVGEKVTISCKSSQSLLYGSSHY
LAWYQQKVGQSPKLLIYWASTRHTGIPDRFIGSGSGTDFTLSLSSVQAEDMADYFCQQYFSTPWTFGAGTRVE
IKSGSGGGSGGGSGGGSQVQLLQSESELVKPGASVKLSCKTSGYTFTDYYIHWVKQKPGQGLEWIGLINPNND
GTNYNQKFQGKATLTADKSSSTAYMELNSLTFDDSVIYFCARASSRLRMARTTSDYYAMDYWGQGIQVTVSSV
DSSGSGGCKPCICTVPEVSSVFIFPPKPKDVLTITLTPKVTCVVVDISKDDPEVQFSWFVDDVEVHTAQTQPR
EEQFNSTFRSVSELPIMHQDWLNGKEFKCRVNSAAFPAPIEKTISKTKGRPKAPQVYTIPPPKEQMAKDKVSL
TCMITDFFPEDITVEWQWNGQPAENYKNTQPIMDTDGSYFVYSKLNVQKSNWEAGNTFTCSVLHEGLHNHHTE
KSLSHSPGK
Igκ-HA-OKT3 VHC-Linker-OKT3 VLC-Myc-PDGFR nucleic acid sequence (SEQ ID NO: 73)
atggagacagacacactcctgctatgggtactgctgctctgggttccaggttccactggtgactatccatatg
atgttccagattatgctggggcccagccggccagatctcaggtgcagctgCAGCAAtctggggctgaactggc
aagacctggggcctcagtgaagatgtcctgcaaggcttctggctacacctttactaggtacacgatgcactgg
gtaaaacagaggcctggacagggtctggaatggattggatacattaatcctagccgtggttatactaattaca
atcagaagttcaaggacaaggccacattgactacagacaaatcctccagcacagcctacatgcaactgagcag
cctgacatctgaggactctgcagtctattactgtgcaAgatattatgatgatcattactgccttgactactgg
ggccaaggcaccACACTCaccgtctcctcaggtggcggtggctccggcggtggtgggtcgggtggcggcggat
ctCAGattgtgCTCacccagtctccagcaatcatgtctgcatctccaggggagaaggttaccatgacctgcag
tgccagctcaagtgtaagttacatgaactggtaccagcagaagtcaggcacctcccccaaaagatggatttat
gacacatccaaactggcttctggagtccctgctcacttcaggggcagtgggtctgggacctcttactctctca
caatcagcggcatggaggctgaagatgctgccacttattactgccagcagtggagtagtaacccattcacgtt
cggctcggggaccaagctggagatcaaTcgtgtcgacgaacaaaaactcatctcagaagaggatctgaatgct
gtgggccaggacacgcaggaggtcatcgtggtgccacactccttgccctttaaggtggtggtgatctcagcca
tcctggccctggtggtgctcaccatcatctcccttatcatcctcatcatgctttggcagaagaagccacgt
(tag)
Igκ-HA-OKT3 VHC-Linker-OKT3 VLC-Myc-PDGFR amino acid sequence (SEQ ID NO: 74)
METDTLLLWVLLLWVPGSTGDYPYDVPDYAGAQPARSQVQLQQSGAELARPGASVKMSCKASGYTFTRYTMHW
VKQRPGQGLEWIGYINPSRGYTNYNQKFKDKATLTTDKSSSTAYMQLSSLTSEDSAVYYCARYYDDHYCLDYW
GQGTTLTVSSGGGGSGGGGSGGGGSQIVLTQSPAIMSASPGEKVTMTCSASSSVSYMNWYQQKSGTSPKRWIY
DTSKLASGVPAHFRGSGSGTSYSLTISGMEAEDAATYYCQQWSSNPFTFGSGTKLEINRVDEQKLISEEDLNA
VGQDTQEVIVVPHSLPFKVVVISAILALVVLTIISLIILIMLWQKKPR
Igκ-HA-OKT3 VHC-Linker-OKT3 VLC-Linker-PDGFR nucleic acid sequence (SEQ ID NO: 75)
atggagacagacacactcctgctatgggtactgctgctctgggttccaggttccactggtgactatccatatg
atgttccagattatgctggggcccagccggccagatctcaggtgcagctgCAGCAAtctggggctgaactggc
aagacctggggcctcagtgaagatgtcctgcaaggcttctggctacacctttactaggtacacgatgcactgg
gtaaaacagaggcctggacagggtctggaatggattggatacattaatcctagccgtggttatactaattaca
atcagaagttcaaggacaaggccacattgactacagacaaatcctccagcacagcctacatgcaactgagcag
cctgacatctgaggactctgcagtctattactgtgcaAgatattatgatgatcattactgccttgactactgg
ggccaaggcaccACACTCaccgtctcctcaggtggcggtggctccggcggtggtgggtcgggtggcggcggat
ctCAGattgtgCTCacccagtctccagcaatcatgtctgcatctccaggggagaaggttaccatgacctgcag
tgccagctcaagtgtaagttacatgaactggtaTcagcagaagtcaggcacctcccccaaaagatggatttat
gacacatccaaactggcttctggagtccctgctcacttcaggggcagtgggtctgggacctcttactctctca
caatcagcggcatggaggctgaagatgctgccacttattactgccagcagtggagtagtaacccattcacgtt
cggctcggggaccaagctggagatcaaTcgtGGCAGTGGgAGTGGgAGTGGgAGTGGgaatgctgtgggccag
gacacgcaggaggtcatcgtggtgccacactccttgccctttaaggtggtggtgatctcagccatcctggccc
tggtggtgctcaccatcatctcccttatcatcctcatcatgctttggcagaagaagccacgt(tag)
Igκ-HA-OKT3 VHC-Linker-OKT3 VLC-Linker-PDGFR amino acid sequence (SEQ ID NO: 76)
METDTLLLWVLLLWVPGSTGDYPYDVPDYAGAQPARSQVQLQQSGAELARPGASVKMSCKASGYTFTRYTMHW
VKQRPGQGLEWIGYINPSRGYTNYNQKFKDKATLTTDKSSSTAYMQLSSLTSEDSAVYYCARYYDDHYCLDYW
GQGTTLTVSSGGGGSGGGGSGGGGSQIVLTQSPAIMSASPGEKVTMTCSASSSVSYMNWYQQKSGTSPKRWIY
DTSKLASGVPAHFRGSGSGTSYSLTISGMEAEDAATYYCQQWSSNPFTFGSGTKLEINRGSGSGSGSGNAVGQ
DTQEVIVVPHSLPFKVVVISAILALVVLTIISLIILIMLWQKKPR
Igκ-HA-OKT3 VHC-Linker-OKT3 VLC-Linker-PDGFR-P2A-hIL12-p35-P2A-hIL12-p40
nucleic acid sequence (SEQ ID NO: 77)
atggagacagacacactcctgctatgggtactgctgctctgggttccaggttccactggtgactatccatatg
atgttccagattatgctggggcccagccggccagatctcaggtgcagctgCAGCAAtctggggctgaactggc
aagacctggggcctcagtgaagatgtcctgcaaggcttctggctacacctttactaggtacacgatgcactgg
gtaaaacagaggcctggacagggtctggaatggattggatacattaatcctagccgtggttatactaattaca
atcagaagttcaaggacaaggccacattgactacagacaaatcctccagcacagcctacatgcaactgagcag
cctgacatctgaggactctgcagtctattactgtgcaAgatattatgatgatcattactgccttgactactgg
ggccaaggcaccACACTCaccgtctcctcaggtggcggtggctccggcggtggtgggtcgggtggcggcggat
ctCAGattgtgCTCacccagtctccagcaatcatgtctgcatctccaggggagaaggttaccatgacctgcag
tgccagctcaagtgtaagttacatgaactggtaTcagcagaagtcaggcacctcccccaaaagatggatttat
gacacatccaaactggcttctggagtccctgctcacttcaggggcagtgggtctgggacctcttactctctca
caatcagcggcatggaggctgaagatgctgccacttattactgccagcagtggagtagtaacccattcacgtt
cggctcggggaccaagctggagatcaaTcgtGGCAGTGGgAGTGGgaatgctgtgggccaggacacgcaggag
gtcatcgtggtgccacactccttgccctttaaggtggtggtgatctcagccatcctggccctggtggtgctca
ccatcatctcccttatcatcctcatcatgctttggcagaagaagccacgtggatctggggccaccaacttttc
attgctcaagcaggcgggcgatgtggaggaaaaccctggccccGGTACCtggccccctgggtcagcctcccag
ccaccgccctcacctgccgcggccacaggtctgcatccagcggctcgccctgtgtccctgcagtgccggctca
gcatgtgtccagcgcgcagcctcctccttgtggctaccctggtcctcctggaccacctcagtttggccagaaa
cctccccgtggccactccagacccaggaatgttcccatgccttcaccactcccaaaacctgctgagggccgtc
agcaacatgctccagaaggccagacaaactctagaattttacccttgcacttctgaagagattgatcatgaag
atatcacaaaagataaaaccagcacagtggaggcctgtttaccattggaattaaccaagaatgagagttgcct
aaattccagagagacctctttcataactaatgggagttgcctggcctccagaaagacctcttttatgatggcc
ctgtgccttagtagtatttatgaagacttgaagatgtaccaggtggagttcaagaccatgaatgcaaagcttc
tgatggatcctaagaggcagatctttctagatcaaaacatgctggcagttattgatgagctgatgcaggccct
gaatttcaacagtgagactgtgccacaaaaatcctcccttgaagaaccggatttttataaaactaaaatcaag
ctctgcatacttcttcatgctttcagaattcgggcagtgactattgatagagtgatgagctatctgaatgctt
ccggatctggggccaccaacttttcattgctcaagcaggcgggcgatgtggaggaaaaccctggcccctgtca
ccagcagttggtcatctcttggttttccctggtttttctggcatctcccctcgtggccatatgggaactgaag
aaagatgtttatgtcgtagaattggattggtatccggatgcccctggagaaatggtggtcctcacctgtgaca
cccctgaagaagatggtatcacctggaccttggaccagagcagtgaggtcttaggctctggcaaaaccctgac
catccaagtcaaagagtttggagatgctggccagtacacctgtcacaaaggaggcgaggttctaagccattcg
ctcctgctgcttcacaaaaaggaagatggaatttggtccactgatattttaaaggaccagaaagaacccaaaa
ataagacctttctaagatgcgaggccaagaattattctggacgtttcacctgctggtggctgacgacaatcag
tactgatttgacattcagtgtcaaaagcagcagaggctcttctgacccccaaggggtgacgtgcggagctgct
acactctctgcagagagagtcagaggggacaacaaggagtatgagtactcagtggagtgccaggaggacagtg
cctgcccagctgctgaggagagtctgcccattgaggtcatggtggatgccgttcacaagctcaagtatgaaaa
ctacaccagcagcttcttcatcagggacatcatcaaacctgacccacccaagaacttgcagctgaagccatta
aagaattctcggcaggtggaggtcagctgggagtaccctgacacctggagtactccacattcctacttctccc
tgacattctgcgttcaggtccagggcaagagcaagagagaaaagaaagatagagtcttcacggacaagacctc
agccacggtcatctgccgcaaaaatgccagcattagcgtgcgggcccaggaccgctactatagctcatcttgg
agcgaatgggcatctgtgccctgcagttag
Igκ-HA-OKT3 VHC-Linker-OKT3 VLC-Linker-PDGFR-P2A-hIL12-p35-P2A-hIL12-p40
amino acid sequence (SEQ ID NO: 78)
METDTLLLWVLLLWVPGSTGDYPYDVPDYAGAQPARSQVQLQQSGAELARPGASVKMSCKASGYTFTRYTMHW
VKQRPGQGLEWIGYINPSRGYTNYNQKFKDKATLTTDKSSSTAYMQLSSLTSEDSAVYYCARYYDDHYCLDYW
GQGTTLTVSSGGGGSGGGGSGGGGSQIVLTQSPAIMSASPGEKVTMTCSASSSVSYMNWYQQKSGTSPKRWIY
DTSKLASGVPAHFRGSGSGTSYSLTISGMEAEDAATYYCQQWSSNPFTFGSGTKLEINRGSGSGNAVGQDTQE
VIVVPHSLPFKVVVISAILALVVLTIISLIILIMLWQKKPRGSGATNFSLLKQAGDVEENPGPGTWPPGSASQ
PPPSPAAATGLHPAARPVSLQCRLSMCPARSLLLVATLVLLDHLSLARNLPVATPDPGMFPCLHHSQNLLRAV
SNMLQKARQTLEFYPCTSEEIDHEDITKDKTSTVEACLPLELTKNESCLNSRETSFITNGSCLASRKTSFMMA
LCLSSIYEDLKMYQVEFKTMNAKLLMDPKRQIFLDQNMLAVIDELMQALNFNSETVPQKSSLEEPDFYKTKIK
LCILLHAFRIRAVTIDRVMSYLNASGSGATNFSLLKQAGDVEENPGPCHQQLVISWFSLVFLASPLVAIWELK
KDVYVVELDWYPDAPGEMVVLTCDTPEEDGITWTLDQSSEVLGSGKTLTIQVKEFGDAGQYTCHKGGEVLSHS
LLLLHKKEDGIWSTDILKDQKEPKNKTFLRCEAKNYSGRFTCWWLTTISTDLTFSVKSSRGSSDPQGVTCGAA
TLSAERVRGDNKEYEYSVECQEDSACPAAEESLPIEVMVDAVHKLKYENYTSSFFIRDIIKPDPPKNLQLKPL
KNSRQVEVSWEYPDTWSTPHSYFSLTFCVQVQGKSKREKKDRVFTDKTSATVICRKNASISVRAQDRYYSSSW
SEWASVPCS
Igκ-OKT3 VHC-Linker-OKT3 VLC-Linker-PDGFR-P2A-hIL12-p35-P2A-hIL12-p40
nucleic acid sequence (SEQ ID NO: 79)
atggagacagacacactcctgctatgggtactgctgctctgggttccaggttccactggtgacggggcccagc
cggccagatctcaggtgcagctgCAGCAAtctggggctgaactggcaagacctggggcctcagtgaagatgtc
ctgcaaggcttctggctacacctttactaggtacacgatgcactgggtaaaacagaggcctggacagggtctg
gaatggattggatacattaatcctagccgtggttatactaattacaatcagaagttcaaggacaaggccacat
tgactacagacaaatcctccagcacagcctacatgcaactgagcagcctgacatctgaggactctgcagtcta
ttactgtgcaAgatattatgatgatcattactgccttgactactggggccaaggcaccACACTCaccgtctcc
tcaggtggcggtggctccggcggtggtgggtcgggtggcggcggatctCAGattgtgCTCacccagtctccag
caatcatgtctgcatctccaggggagaaggttaccatgacctgcagtgccagctcaagtgtaagttacatgaa
ctggtaTcagcagaagtcaggcacctcccccaaaagatggatttatgacacatccaaactggcttctggagtc
cctgctcacttcaggggcagtgggtctgggacctcttactctctcacaatcagcggcatggaggctgaagatg
ctgccacttattactgccagcagtggagtagtaacccattcacgttcggctcggggaccaagctggagatcaa
TcgtGGCAGTGGgAGTGGgaatgctgtgggccaggacacgcaggaggtcatcgtggtgccacactccttgccc
tttaaggtggtggtgatctcagccatcctggccctggtggtgctcaccatcatctcccttatcatcctcatca
tgctttggcagaagaagccacgtggatctggggccaccaacttttcattgctcaagcaggcgggcgatgtgga
ggaaaaccctggccccGGTACCtggccccctgggtcagcctcccagccaccgccctcacctgccgcggccaca
ggtctgcatccagcggctcgccctgtgtccctgcagtgccggctcagcatgtgtccagcgcgcagcctcctcc
ttgtggctaccctggtcctcctggaccacctcagtttggccagaaacctccccgtggccactccagacccagg
aatgttcccatgccttcaccactcccaaaacctgctgagggccgtcagcaacatgctccagaaggccagacaa
actctagaattttacccttgcacttctgaagagattgatcatgaagatatcacaaaagataaaaccagcacag
tggaggcctgtttaccattggaattaaccaagaatgagagttgcctaaattccagagagacctctttcataac
taatgggagttgcctggcctccagaaagacctcttttatgatggccctgtgccttagtagtatttatgaagac
ttgaagatgtaccaggtggagttcaagaccatgaatgcaaagcttctgatggatcctaagaggcagatctttc
tagatcaaaacatgctggcagttattgatgagctgatgcaggccctgaatttcaacagtgagactgtgccaca
aaaatcctcccttgaagaaccggatttttataaaactaaaatcaagctctgcatacttcttcatgctttcaga
attcgggcagtgactattgatagagtgatgagctatctgaatgcttccggatctggggccaccaacttttcat
tgctcaagcaggcgggcgatgtggaggaaaaccctggcccctgtcaccagcagttggtcatctcttggttttc
cctggtttttctggcatctcccctcgtggccatatgggaactgaagaaagatgtttatgtcgtagaattggat
tggtatccggatgcccctggagaaatggtggtcctcacctgtgacacccctgaagaagatggtatcacctgga
ccttggaccagagcagtgaggtcttaggctctggcaaaaccctgaccatccaagtcaaagagtttggagatgc
tggccagtacacctgtcacaaaggaggcgaggttctaagccattcgctcctgctgcttcacaaaaaggaagat
ggaatttggtccactgatattttaaaggaccagaaagaacccaaaaataagacctttctaagatgcgaggcca
agaattattctggacgtttcacctgctggtggctgacgacaatcagtactgatttgacattcagtgtcaaaag
cagcagaggctcttctgacccccaaggggtgacgtgcggagctgctacactctctgcagagagagtcagaggg
gacaacaaggagtatgagtactcagtggagtgccaggaggacagtgcctgcccagctgctgaggagagtctgc
ccattgaggtcatggtggatgccgttcacaagctcaagtatgaaaactacaccagcagcttcttcatcaggga
catcatcaaacctgacccacccaagaacttgcagctgaagccattaaagaattctcggcaggtggaggtcagc
tgggagtaccctgacacctggagtactccacattcctacttctccctgacattctgcgttcaggtccagggca
agagcaagagagaaaagaaagatagagtcttcacggacaagacctcagccacggtcatctgccgcaaaaatgc
cagcattagcgtgcgggcccaggaccgctactatagctcatcttggagcgaatgggcatctgtgccctgcagt
tag
Igκ-OKT3 VHC-Linker-OKT3 VLC-Linker-PDGFR-P2A-hIL12-p35-P2A-hIL12-p40 amino
acid sequence (SEQ ID NO: 80)
METDTLLLWVLLLWVPGSTGDGAQPARSQVQLQQSGAELARPGASVKMSCKASGYTFTRYTMHWVKQRPGQGL
EWIGYINPSRGYTNYNQKFKDKATLTTDKSSSTAYMQLSSLTSEDSAVYYCARYYDDHYCLDYWGQGTTLTVS
SGGGGSGGGGSGGGGSQIVLTQSPAIMSASPGEKVTMTCSASSSVSYMNWYQQKSGTSPKRWIYDTSKLASGV
PAHFRGSGSGTSYSLTISGMEAEDAATYYCQQWSSNPFTFGSGTKLEINRGSGSGNAVGQDTQEVIVVPHSLP
FKVVVISAILALVVLTIISLIILIMLWQKKPRGSGATNFSLLKQAGDVEENPGPGTWPPGSASQPPPSPAAAT
GLHPAARPVSLQCRLSMCPARSLLLVATLVLLDHLSLARNLPVATPDPGMFPCLHHSQNLLRAVSNMLQKARQ
TLEFYPCTSEEIDHEDITKDKTSTVEACLPLELTKNESCLNSRETSFITNGSCLASRKTSFMMALCLSSIYED
LKMYQVEFKTMNAKLLMDPKRQIFLDQNMLAVIDELMQALNFNSETVPQKSSLEEPDFYKTKIKLCILLHAFR
IRAVTIDRVMSYLNASGSGATNFSLLKQAGDVEENPGPCHQQLVISWFSLVFLASPLVAIWELKKDVYVVELD
WYPDAPGEMVVLTCDTPEEDGITWTLDQSSEVLGSGKTLTIQVKEFGDAGQYTCHKGGEVLSHSLLLLHKKED
GIWSTDILKDQKEPKNKTFLRCEAKNYSGRFTCWWLTTISTDLTFSVKSSRGSSDPQGVTCGAATLSAERVRG
DNKEYEYSVECQEDSACPAAEESLPIEVMVDAVHKLKYENYTSSFFIRDIIKPDPPKNLQLKPLKNSRQVEVS
WEYPDTWSTPHSYFSLTFCVQVQGKSKREKKDRVFTDKTSATVICRKNASISVRAQDRYYSSSWSEWASVPCS
hIL12-p35-P2A-hIL12-p40-P2A-hCXCL9 nucleic acid sequence (SEQ ID NO: 81)
atgtggccccctgggtcagcctcccagccaccgccctcacctgccgcggccacaggtctgcatccagcggctc
gccctgtgtccctgcagtgccggctcagcatgtgtccagcgcgcagcctcctccttgtggctaccctggtcct
cctggaccacctcagtttggccagaaacctccccgtggccactccagacccaggaatgttcccatgccttcac
cactcccaaaacctgctgagggccgtcagcaacatgctccagaaggccagacaaactctcgaattttaccctt
gcacttctgaagagattgatcatgaagatatcacaaaagataaaaccagcacagtggaggcctgtttaccatt
ggaattaaccaagaatgagagttgcctaaattccagagagacctctttcataactaatgggagttgcctggcc
tccagaaagacctcttttatgatggccctgtgccttagtagtatttatgaagacttgaagatgtaccaggtgg
agttcaagaccatgaatgcaaagcttctgatggaccctaagaggcaaatcttcctagatcaaaacatgctggc
agttattgatgagctgatgcaggccctgaatttcaacagtgagactgtgccacaaaaatcctcccttgaagaa
ccggatttctacaagactaaaatcaagctctgcatacttcttcatgctttcagaatccgggcagtgactattg
atagagtgatgagctatctgaatgcttccGCGGCCGCAggatctggggccaccaacttttcattgctcaagca
ggcCggcgatgtggaggaaaaccctggccccGGATCCtgtcaccagcagttggtcatctcttggttttccctg
gtttttctggcatctcccctcgtggccatatgggaactgaagaaagatgtttatgtcgtagaattggattggt
atccggatgcccctggagaaatggtggtcctcacctgtgacacccctgaagaagatggtatcacctggacctt
ggaccagagcagtgaggtcttaggctctggcaaaaccctgaccatccaagtcaaagagtttggagatgctggc
cagtacacctgtcacaaaggaggcgaggttctaagccattcgctcctgctgcttcacaaaaaggaagatggaa
tttggtccactgatattttaaaggaccagaaagaacccaaaaataagacctttctaagatgcgaggccaagaa
ttattctggacgtttcacctgctggtggctgacgacaatcagtactgatttgacattcagtgtcaaaagcagc
agaggctcttctgacccccaaggggtgacgtgcggagctgctacactctctgcagagagagtcagaggggaca
acaaggagtatgagtactcagtggagtgccaggaggacagtgcctgcccagctgctgaggagagtctgcccat
tgaggtcatggtggatgccgttcacaagctcaagtatgaaaactacaccagcagcttcttcatcagggacatc
atcaaacctgacccacccaagaacttgcagctgaagccattaaagaaCtctcggcaggtggaggtcagctggg
agtaccctgacacctggagtactccacattcctacttctccctgacattctgcgttcaggtccagggcaagag
caagagagaaaagaaagatagagtcttcacggacaagacctcagccacggtcatctgccgcaaaaatgccagc
attagcgtgcgggcccaggaccgctactatagctcatcttggagcgaatgggcatctgtgccctgcagttCgT
CTAGAggatctggggccaccaacttttcattgctcaagcaggcgggcgatgtggaggaaaaccctggccccaa
gaaaagtggtgttcttttcctcttgggcatcatcttgctggttctgattggagtgcaaggaaccccagtagtg
agaaagggtcgctgttcctgcatcagcaccaaccaagggactatccacctacaatccttgaaagaccttaaac
aatttgccccaagcccttcctgcgagaaaattgaaatcattgctacactgaagaatggagttcaaacatgtct
aaacccagattcagcagatgtgaaggaactgattaaaaagtgggagaaacaggtcagccaaaagaaaaagcaa
aagaatgggaaaaaacatcaaaaaaagaaagttctgaaagttcgaaaatctcaacgttctcgtcaaaagaaga
ctacataa
hIL12-p35-P2A-hIL12-p40-P2A-hCXCL9 amino acid sequence (SEQ ID NO: 82)
MWPPGSASQPPPSPAAATGLHPAARPVSLQCRLSMCPARSLLLVATLVLLDHLSLARNLPVATPDPGMFPCLH
HSQNLLRAVSNMLQKARQTLEFYPCTSEEIDHEDITKDKTSTVEACLPLELTKNESCLNSRETSFITNGSCLA
SRKTSFMMALCLSSIYEDLKMYQVEFKTMNAKLLMDPKRQIFLDQNMLAVIDELMQALNFNSETVPQKSSLEE
PDFYKTKIKLCILLHAFRIRAVTIDRVMSYLNASAAAGSGATNFSLLKQAGDVEENPGPGSCHQQLVISWFSL
VFLASPLVAIWELKKDVYVVELDWYPDAPGEMVVLTCDTPEEDGITWTLDQSSEVLGSGKTLTIQVKEFGDAG
QYTCHKGGEVLSHSLLLLHKKEDGIWSTDILKDQKEPKNKTFLRCEAKNYSGRFTCWWLTTISTDLTFSVKSS
RGSSDPQGVTCGAATLSAERVRGDNKEYEYSVECQEDSACPAAEESLPIEVMVDAVHKLKYENYTSSFFIRDI
IKPDPPKNLQLKPLKNSRQVEVSWEYPDTWSTPHSYFSLTFCVQVQGKSKREKKDRVFTDKTSATVICRKNAS
ISVRAQDRYYSSSWSEWASVPCSSSRGSGATNFSLLKQAGDVEENPGPKKSGVLFLLGIILLVLIGVQGTPVV
RKGRCSCISTNQGTIHLQSLKDLKQFAPSPSCEKIEIIATLKNGVQTCLNPDSADVKELIKKWEKQVSQKKKQ
KNGKKHQKKKVLKVRKSQRSRQKKTT
PDGFRβ transmembrane domain (SEQ ID NO: 83)
VVISAILALVVLTVISLIILI
PDGFRβ transmembrane domain (SEQ ID NO: 84)
VVISAILALVVLTIISLIILI
PDGFRα transmembrane domain (SEQ ID NO: 85)
AAVLVLLVIVIISLIVLVVIW
PDGFRα transmembrane domain (SEQ ID NO: 86)
AAVLVLLVIVIVSLIVLVVIW
PDGFRβ transmembrane domain (SEQ ID NO: 87)
tggtgatctcagccatcctggccctggtggtgctcaccatcatctcccttatcatcctcatc
PDGFRβ transmembrane domain (SEQ ID NO: 88)
gtggtgatctcagccatcctggccctggtggtgctcaccatcatctcccttatcatcctcatc
PDGFRα transmembrane domain (SEQ ID NO: 89)
gctgcagtcctggtgctgttggtgattgtgatcatctcacttattgtcctggttgtcatttggaa

Although the invention has been described in detail for purposes of clarity of understanding, certain modifications may be practiced within the scope of the appended claims. All publications, accession numbers, web sites, patent documents and the like cited in this application are hereby incorporated by reference in their entirety for all purposes to the same extent as if each were so individually denoted. To the extent different information is associated with a citation at different times, the information present as of the effective filing date of this application is meant. Unless otherwise apparent from the context any element, embodiment, step, feature or aspect of the invention can be performed in combination with any other.

### EXAMPLES

### CXCL9

Example 1. CXCL9 plasmid construction. Mouse CXCL9 (mCXCL9) or human CXCL9 (hCXCL9) nucleic acid sequence was cloned into an expression vector using standard molecular biology techniques to. Alternatively, mCXCL9 or hCXCL9 was cloned downstream of mouse (mIL12-2A) or human (hIL12-2A) IL12 p35-P2A-IL12 p40 to yield mIL12˜mCXCL9 and hIL12˜hCXCL9 (FIG. 1A-B). IL12 p35-P2A-IL12 p40 constructs were made essentially as described in WO2017/106795 or WO2018/229696.

The resulting plasmids contained IL-12 p35, IL-12 p40 and CXCL9, all expressed from the same promoter, with intervening exon skipping (P2A) motifs to allow all three proteins to be expressed from a single polycistronic message. Similar methods were sued to make mCXCL9˜mCherry

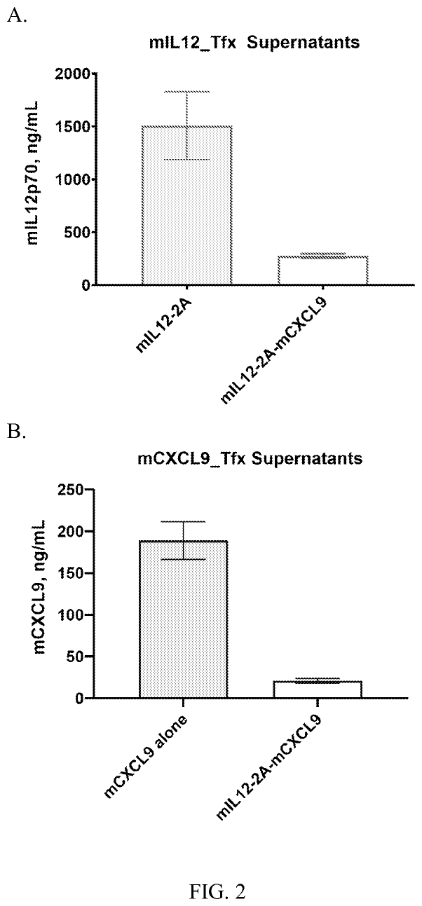

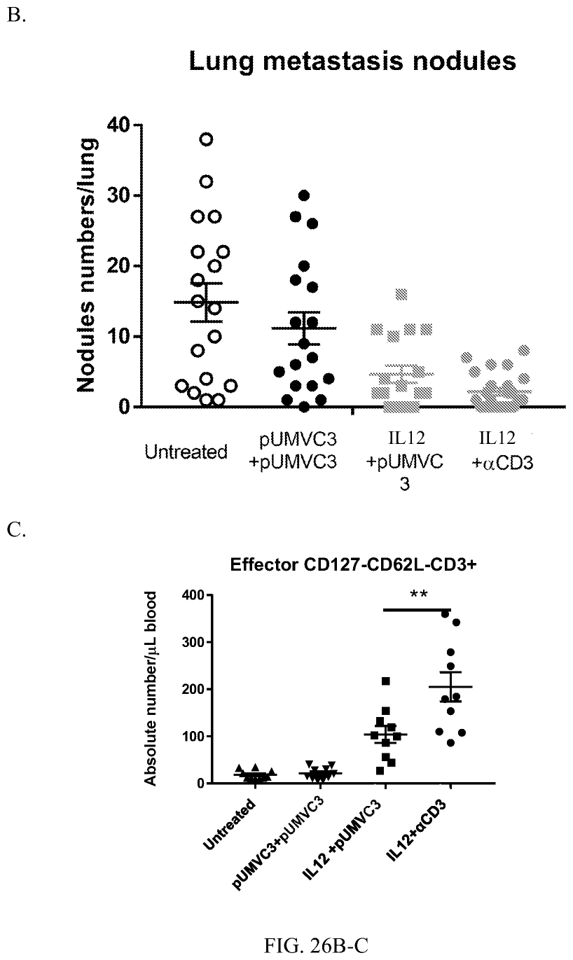

Example 2. Protein expression. The mIL12-2A, mCXCL9, and mIL12˜mCXCL9 expression vectors were transfected into HEK293 cells in vitro. 96 h after transfection, supernatants were collected and IL12 and CXCL9 protein expression were assayed by ELISA. The results, shown in FIG. 2 show that, while expression was decreased in the cells transfected with the mIL12˜mCXCL9 expression vector, detectable levels of both IL12 and CXCL9 were produced. FIG. 27 shows high levels of secreted hIL12 and hCXCL9 in cells transfected with hIL-12˜hCXCL9 expression vector.

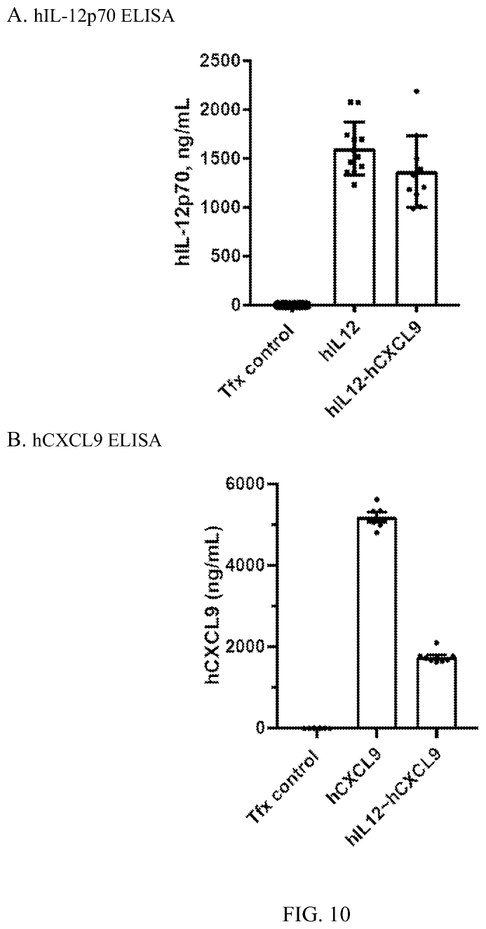

Similarly, hIL12-2A, hCXCL9, and hIL12˜hCXCL9 expression vectors were transfected into HEK293 cells in vitro. 96 h after transfection, supernatants were collected and IL12 and CXCL9 protein expression were assayed by ELISA. hIL12 was expressed nearly equally from both the hIL12-2A (1.59 μg/mL) and hIL12˜hCXCL9 (1.37 μg/mL) expression vectors (FIG. 10A). Decreased, but still substantial levels of hCXCL9 was expressed in cells transfected with the hIL12˜hCXCL9 expression vector (1.75 μg/mL) compared to cells transfected with the hCXCL9 expression vector (5.19 μg/mL) (FIG. 10B).

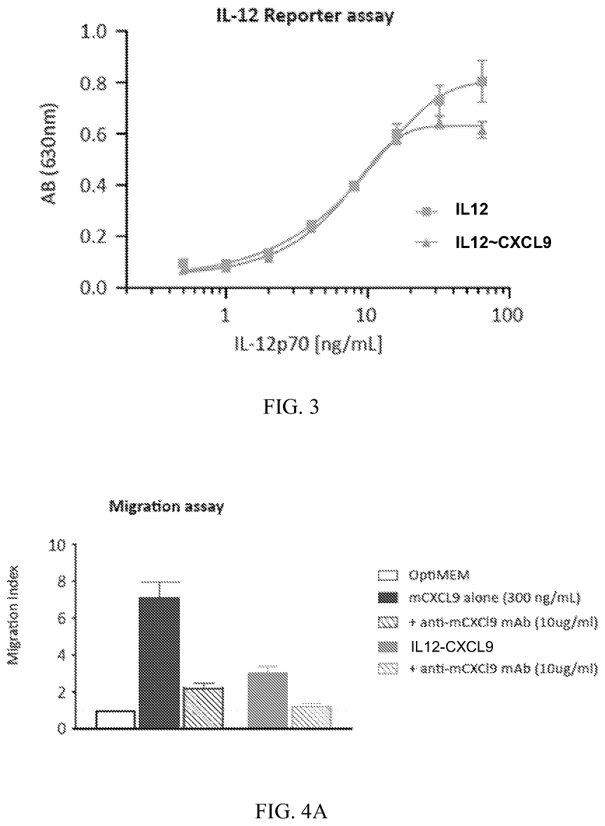

mIL12 protein produced from the mIL12˜mCXCL9 expression vector was further tested for activity. mIL12 produced from cells transfected with the mIL12-2A or mIL12˜mCXCL9 expression vectors was incubated with HEK-Blue IL-12 cells. HEK-Blue IL-12 cells are used to detect bioactive human and mouse IL-12. HEK-Blue IL-12 cells are used to validate the functionality of recombinant native or engineered human or mouse IL-12. Functional IL-12 binds to IL-12 receptor in HEK-Blue IL-12 cells and activates a STAT-4 pathway and a STAT4-inducible SEAP reporter gene. SEAP expression is then assayed. The response ratio was calculated by dividing the OD at 630 nm for treated cells by the OD at 630 nm for untreated cells. The results, shown in FIG. 3, demonstrate that IL-12, produced from either the mIL12-2A or the mIL12˜mCXCL9 expression vectors is functional.

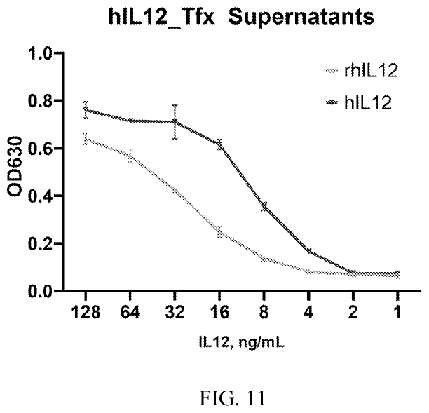

Similarly, hIL12 protein produced from the hIL12-hCXCL9 expression vector was also tested for activity. hIL12 produced from cells transfected with the hIL12-hCXCL9 expression vector was incubated with HEK-Blue IL-12 cells. The results, shown in FIG. 11, demonstrate that IL-12, produced from the hIL12-2A expression vector, is functional.

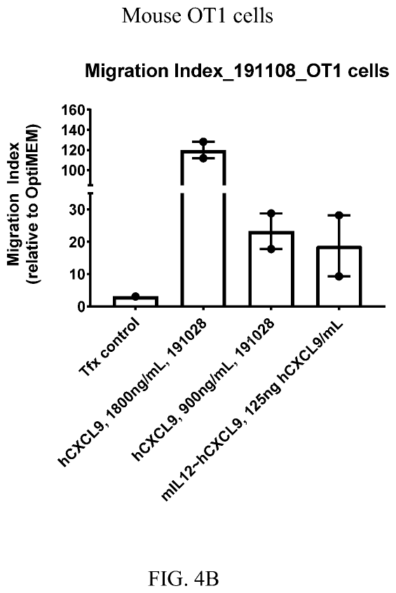

Example 3. CXCL9-induced migration of T cells in vitro. Mammalian (HEK293) cells were transfected with CXCL9 expression vectors (CXCL9 or IL12˜CXCL9). OT-I mouse splenocytes were pulsed with 1 μg/mL SIINFEKL peptide for 24 h, then allowed to recover for 72 h. The CXCL9 transfected cells were then assayed for the induction of chemotaxis of the SIINFEKL-pulsed OT-I splenocytes through polycarbonate membranes with 5.0-micron pores. Migration index was defined as the number of observed chemotactic cells, normalized to the number of cells that passively migrated through the membrane in the OptiMEM negative control. Results are shown in FIGS. 4A, 4B, and 4C. mCXCL9, produced from mCXCL9 and mIL12˜mCXCL9 expression vectors caused about 7-fold and about 3-fold increases in chemotactic cells, respectively. The increase in chemotaxis was inhibited by the addition of CXCL9 neutralizing antibodies, indicating the effect was dependent on mCXCL9.

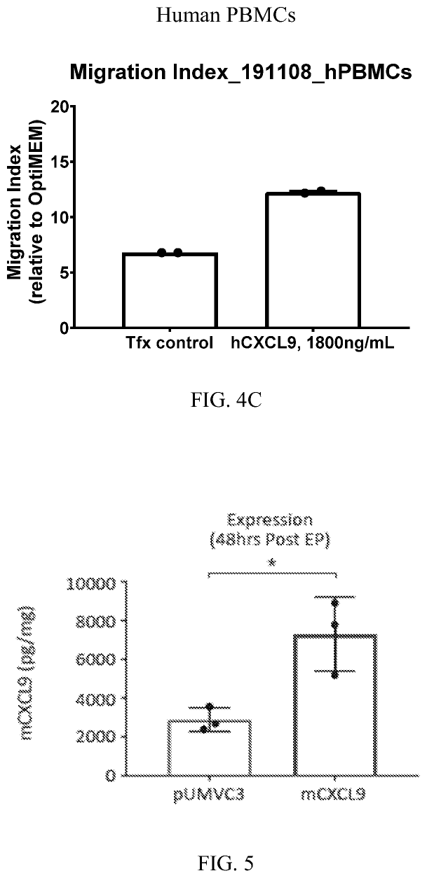

Example 4. In vivo expression of mCXCL9. CT-26 (colon carcinoma) tumors were implanted in mice. Tumors were subsequently treated with IT-EP pUMCV3 control vector or IT-EP mCXCL9 expression vector. 48 h after IT-EP, tumors were homogenized and assay for CXCL9 expression by ELISA (DuoSet ELISA DY392; n=3; *P<0.05; T test with Welch correction). The results in FIG. 5 show that the IT-EP treated tumors expressed CXCL9.

Example 5. Tumor regression in mice treated with mIL12-2A and mCXCL9. Mice were implanted with tumor cells. Anesthetized mice were subcutaneously injected with cells into the right and/or left flank. Tumor growth was monitored by digital caliper measurements until average tumor volume reached ˜100 mm³.

Tumors were treated on day 0 with IT-EP control vector or IT-EP IL12-2A expression vector and on days 4 and 7 with IT-EP control vector or IT-EP CXCL9 (optionally with mcherry reported protein). Tumor volumes and survival were monitored. Mice were euthanized when the total tumor burden of the primary and contralateral reached 2000 mm³.

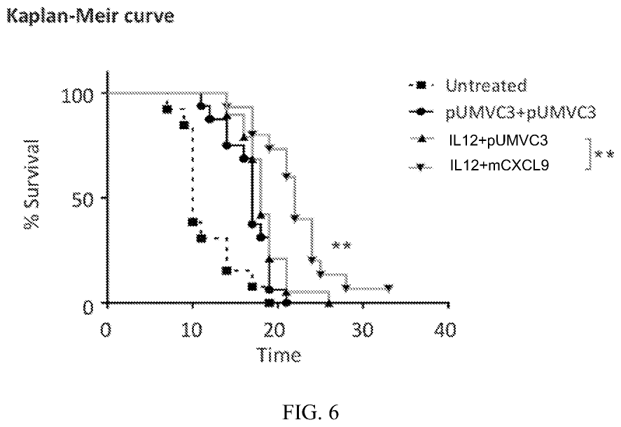

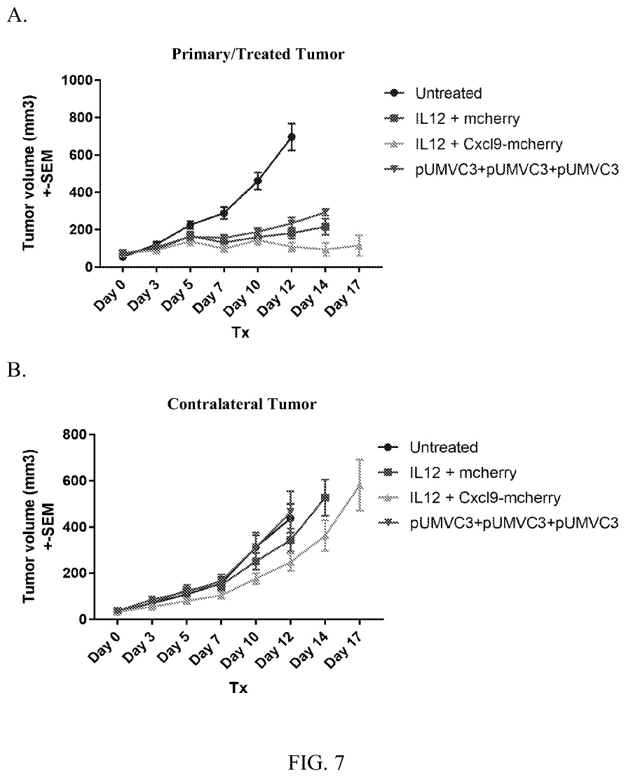

The data, shown in FIG. 6, show that mice treated with IT-EP mIL12-2A plus mCXCL9 therapy showed increased survival compared to untreated mice, mice treated with control vehicle, or mice treated with IT-EP mIL12-2A alone. Tumor bearing mice treated with IT-EP mIL12-2A plus mCXCL9-mCherry therapy also showed decreased primary (treated) and contralateral (untreated) tumor progression (FIG. 7A-B).

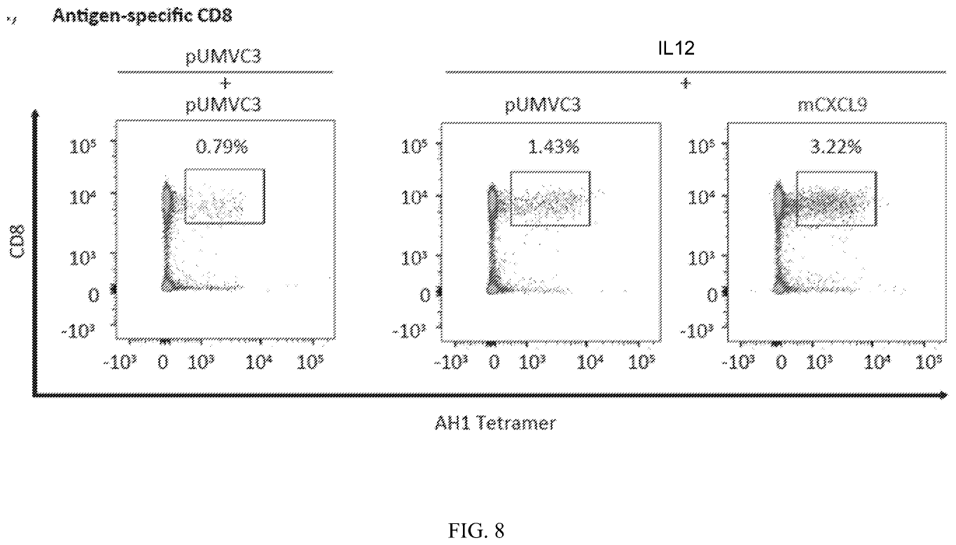

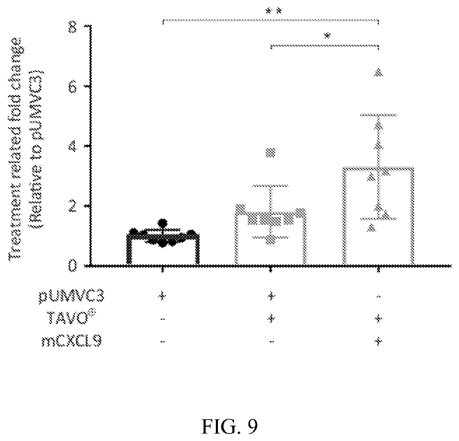

Example 6. IT-EP IL12-2A+IT-EP CXCL9 drives systemic expansion of antigen specific CD8 and short-lived effector cells (SLECs). On day −8, mice were implanted with tumor as described above. On day 0, tumors were treated with IT-EP mIL12-2A. On days 4 and 7, mice were treated with control plasmid or mCXCL9 (n=3/group) using IT-EP as described above. On day 9, spleens were harvested and CD3⁺CD8⁺ cells analyzed by FACS. FIG. 8 shows that CD3⁺ T cell populations were significantly increased in mice treated with IL12-2A+CXCL9. Fold increase in the number of AH1+ CD8+ T cells is shown in FIG. 9.

Example 7. Intratumoral CXCL9 synergizes with IL-12 to modulate the tumor microenvironment, expand antigen-specific T cells, and control contralateral tumor growth. A mouse model was used to evaluate intratumoral expression post electroporation.

CT26 tumors were implanted in mice on day −7. For NanoString analysis and flow based assays single, tumor model was used. Mice were treated on day 1 with IT-EP with a suboptimal dose of IL12-2A followed by treatment on days 4 and 7 with IT-EP using 100 μg of either mCXCL9 or pUMVC3. Tumor and immune response were then monitored. Tumor and splenocytes were harvested 2 days after last EP (i.e., Day 9) for NanoString and flow based analysis. Alternatively, tumor volumes were measured three times a week for regression/survival studies. Gene expression changes in electroporated CT26 lesions were assessed by NanoString nCounter® technology. Intratumoral expression of mCXCL9 was confirmed using ELISA for mCXCL9 48 hrs post-electroporation in tumor lysates from mice bearing CT26 tumors (n=3; *P<0.05; T test with Welch correction).

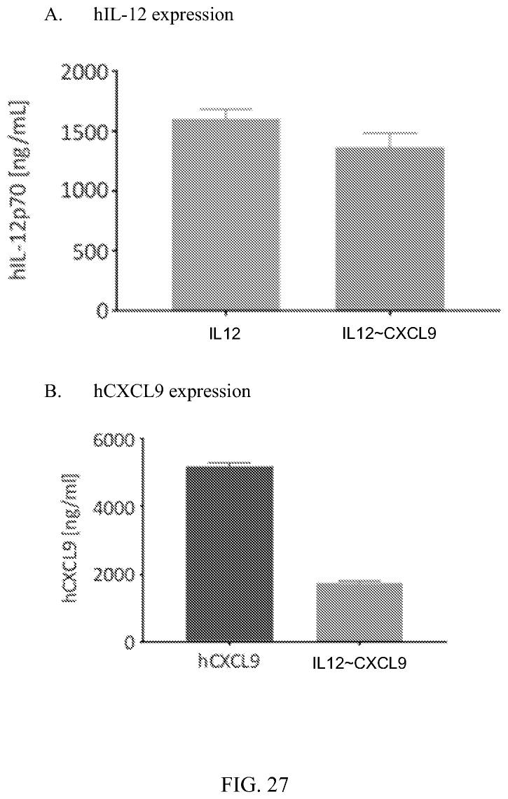

Volcano plots displaying p-values and log 2 fold change for each gene were generated in mice treated with CXCL9 alone or CXCL9 in combination with IL12-2A (FIG. 28A). Analysis of cell type scores showed in increase in Cytotoxic immune cells in response to treatment with either CXCL9 or IL12-2A. A synergistic increase in Cytotoxic immune cell scores was further seen when CXCL9 was administered in combination with IL12-2A. ‘Cytotoxic immune cells’ cell type scores are shown in FIG. 28B.

Flow cytometric analysis of was used to analyze splenocytes in treated mice. Antigen specific AH1+ CD8+ T cells were measured via tetramer analysis (Immudex). Cells are gated on Singlets<Live<CD3+CD4− splenocytes (FIG. 8). The fold increase in the number of AH1+ CD8+ T cells compared to empty vector control (N=2 independent experiments with 3-5 animals/group; *P<0.05, **P<0.005; One way ANOVA). In mice treated with control plasmid only, 0.79% of AH1 tetramer were CD8⁺. In mice treated with IT-EP IL12-2A, 1.43% of AH1 tetramer were CD8⁺. In mice treated with IT-EP IL12-2A and CXCL9, 3.22% of AH1 tetramer were CD8+. Fold increase in the number of AH1+ CD8+ T cells is shown in FIG. 9.

The results show that IT-EP CXCL9 can substantially enhance anti-tumor immune response in animal previously treated with a suboptimal dose of IT-EP IL12-2A.

CD3 half-BiTE

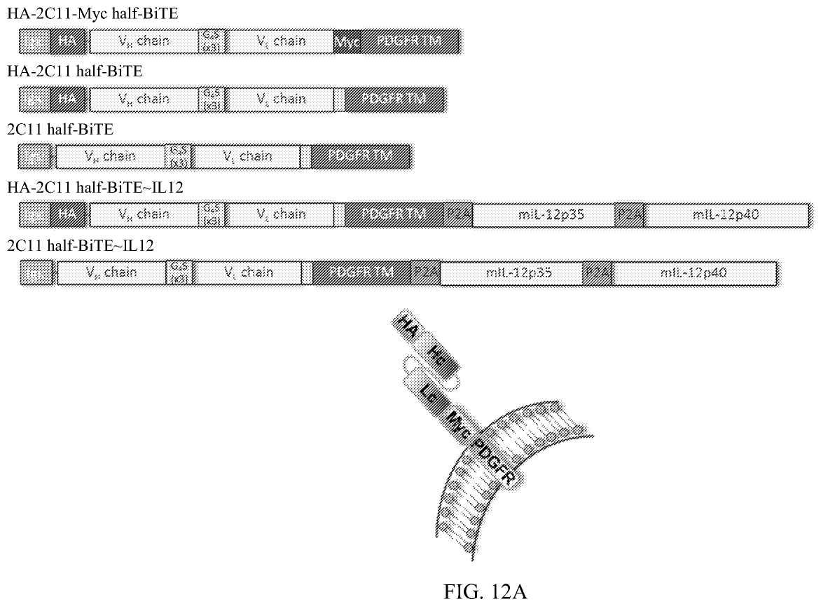

Example 8. The Half-BiTE expression cassettes were made in a manner similar to that described above for the generation of CXCL9 plasmids (FIGS. 12A and 12B).

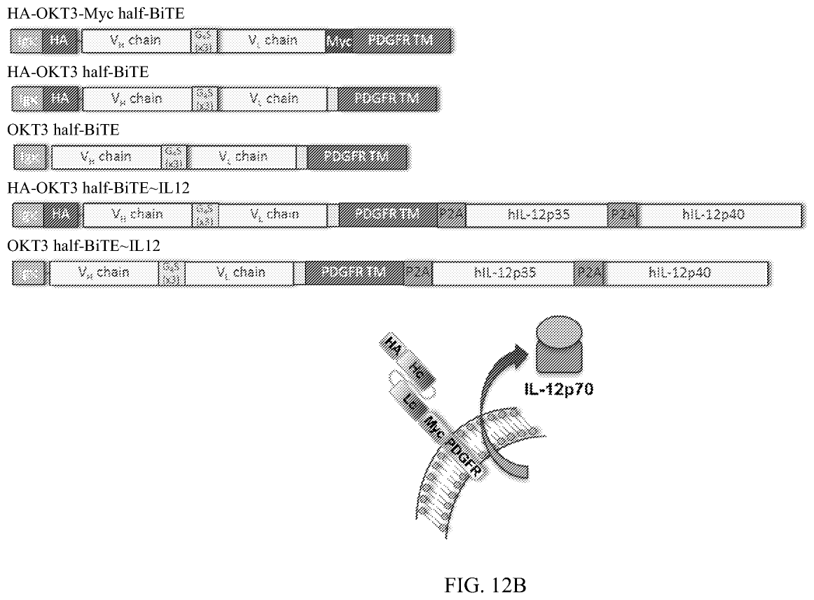

Example 9. Protein expression. The OKT3 scFv and 2C11 scFv, expression vectors were transfected into HEK293 cells in vitro. HA-2C11 scFv and HA-2C11 scFv˜mIL12 were transfected into B16-F10 tumor cells. 24 h after transfection, supernatants were collected, and proteins were separated by gel electrophoresis. CD3 scFv, Cadherin (membrane protein) and Hsp90 were detected by Western blot analysis. The results, shown in FIG. 13 show that the expression vectors expressed the CD3 scFv protein. The CD3 scFv protein was predominantly located in the membrane fraction.

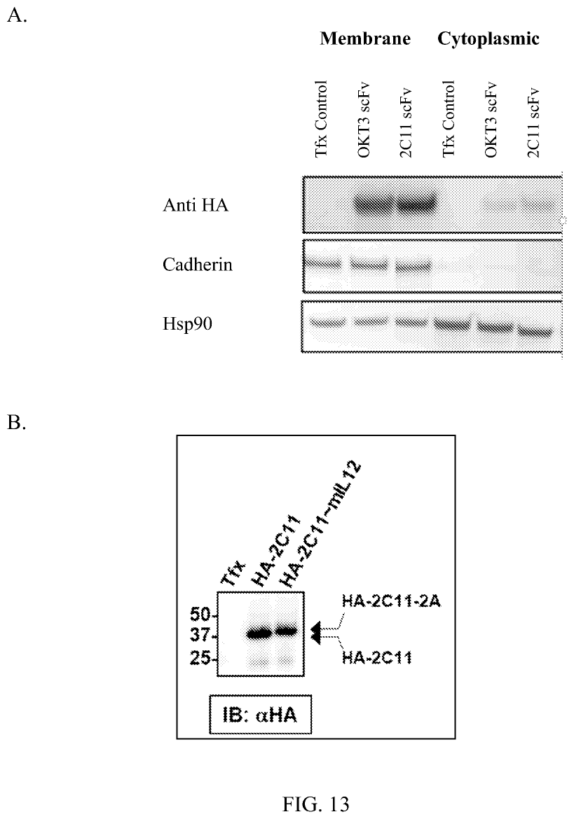

HA-OKT3 scFv, OKT3 scFv˜hIL12 expression vectors were transfected into HEK293 cells in vitro. 72 h after transfection, cells were analyzed by FACS to detect CD3 scFv (FIG. 14A-C). HA-2C11 scFv and HA-2C11 scFv˜mIL12 expression vectors were transfected into B16-F10 cells. Cells were analyzed by FACS to detect surface expression of CD3 scFv (FIG. 14D). Expression of IL12 from IL12-2A and HA-2C11 scFv˜mIL12 expression vectors is shown in FIG. 14E.

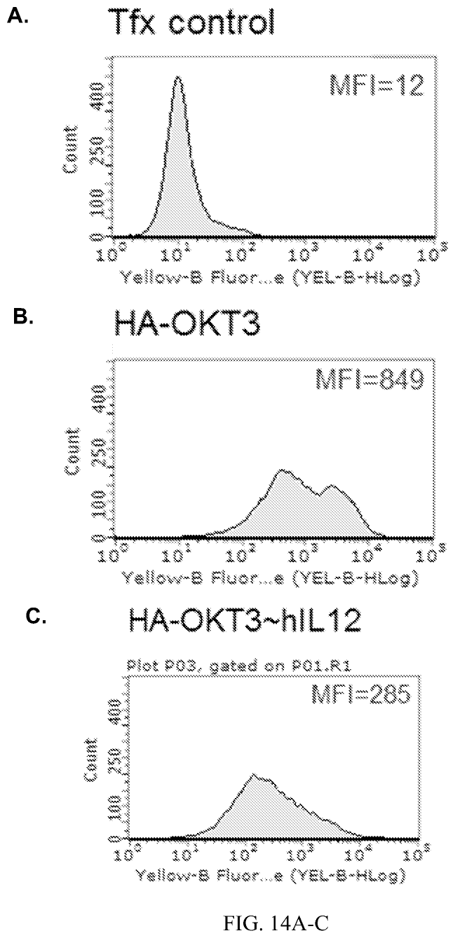

HA-OKT3 scFv˜hIL12 and OKT3 scFv˜hIL12 expression vectors were transfected into HEK293 cells in vitro. 72 h after transfection cells supernatant was collected and assayed for IL12p70 by ELISA. The results confirm that cells transfected with the HA-OKT3 scFv˜hIL12 and OKT3 scFv˜hIL12 expression vectors express and secrete hIL12p70 (FIG. 15).

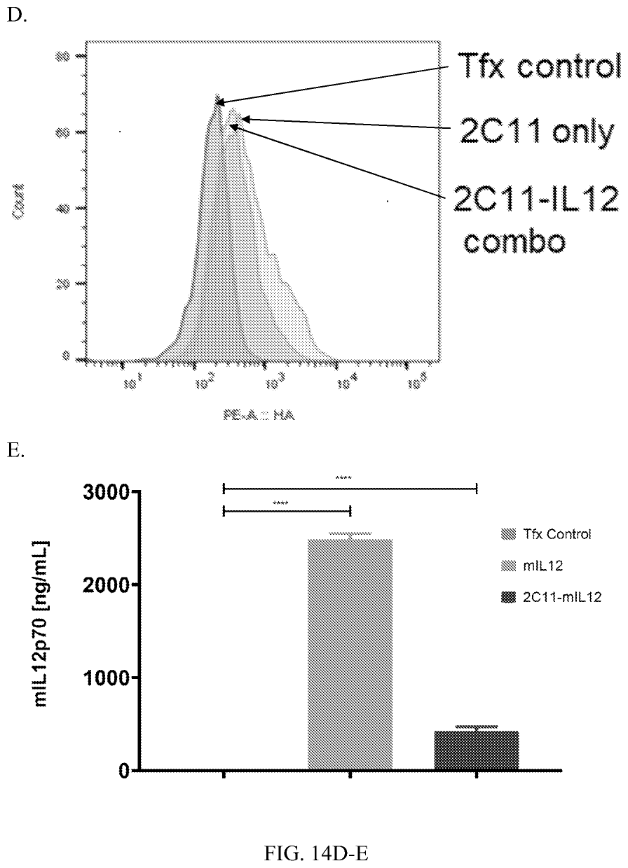

In vivo expression: Mice were inoculated with B16F10 melanoma cells or 4T1 breast cancer cells on day −7. On day 0, tumors were treated with IT-EP HA-2C11 scFv˜hIL12 (FIG. 16). FIG. 16A-B show the CD3 half-BiTE is expressed on the surface of melanoma and breast cancer tumors following IT-EP. FIG. 16C shows that following IT-EP of HA-OKT3 scFv˜hIL12, the expression vector also expresses IL-12.

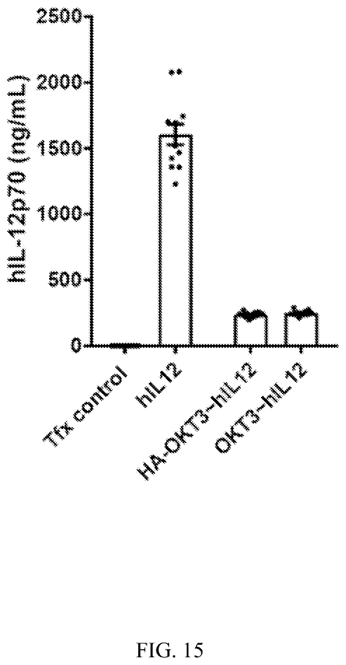

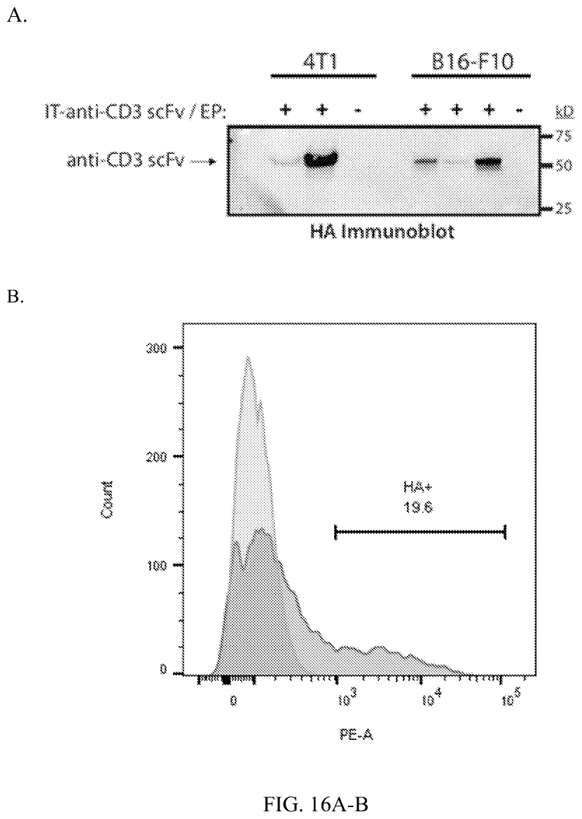

Example 10. In vitro Functional assay. B16F10 cells were transfected in vitro with control vector and 2C11 scFv expression vector with or without recombinant mouse IL12. Transfected B16F10 cells were then co-cultured with naïve mouse splenocytes for 24, 48, or 72 hours. Following co-culture, supernatants were assayed for IFNγ and cell proliferation was evaluated by FACS. Plate bound anti-CD3 was used as a positive control. The results, shown in FIG. 17, show that IFNγ expression was substantially increased when splenocytes were co-cultured with B16F10 expressing 2C11 scFv. FACS analyses were performed to analyze proliferation of CFSE labeled CD3+CD45+ T cells following co-culture of naïve mouse splenocytes with B16F10 cells transfected in vitro with control vector (Tfx control), 2C11 scFv expression vector with or without recombinant mouse IL12, or with plate bound anti-CD3 (positive control) (FIG. 18).

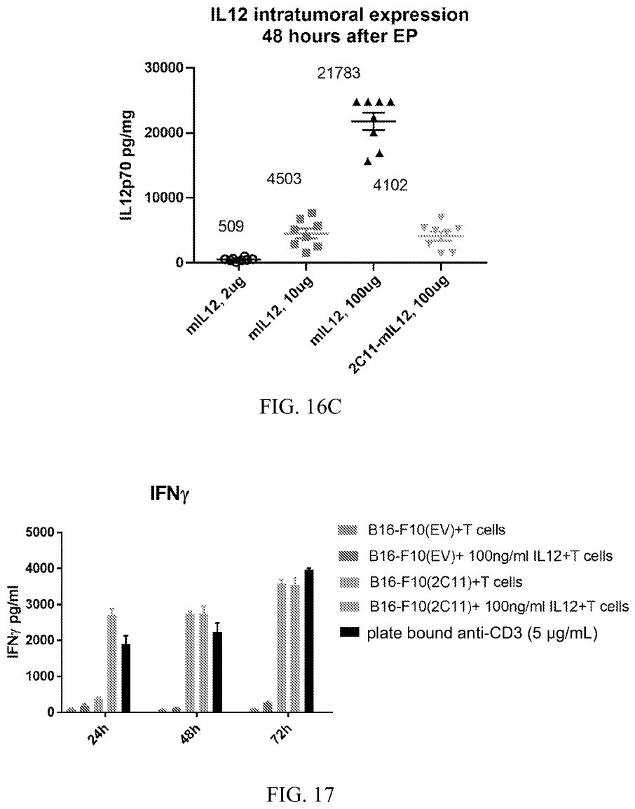

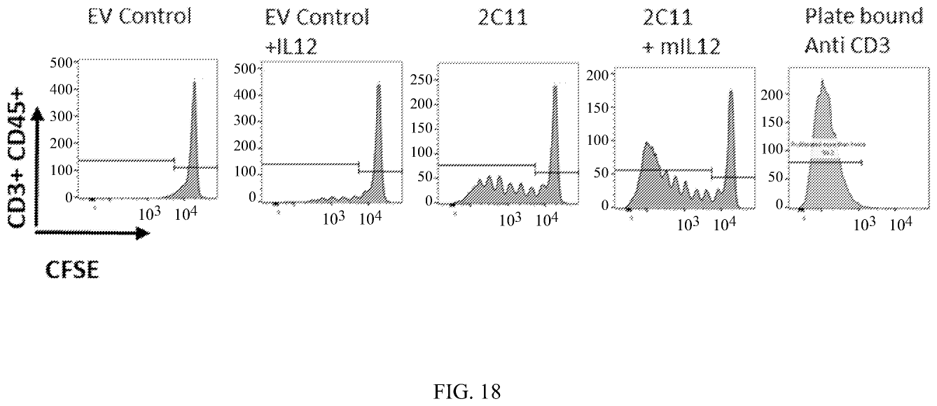

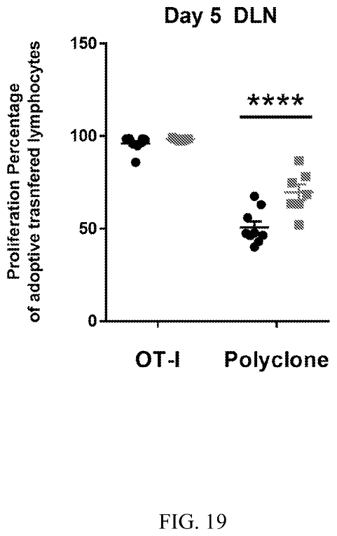

Example 11. In vivo functional assay. On day −9, B16-OVA cells were implanted in mice (n=8/group). On day 0, tumors were treated by IT-EP with 2C11 scFv expression vector or empty vector (negative control). On day 0, mice were also implanted, by adoptive transfer, with a 1:1 mix of OT-1 (GFP) CD8⁺ cells T cells and naïve mouse lymphocytes. On day 5, adoptive transferred T cell proliferation in spleen and draining lymph node (DLN) were examined by FACS. Endogenous T cell populations and SIINFEKL expression in tumor infiltrating lymphocytes (TILs) were also examined by FACS. An increase in polyclonal T cell proliferation in DLN was observed in mice treated with IT-EP 2C11 scFv (FIG. 19). Increases in OT-1 and polyclonal T cell populations were also observed in splenocytes in mice treated with IT-EP 2C11 scFv. An increase CD8+ T cells in CD45.1+ live cells in TILs was observed in B16-OVA tumor model mice treated with 2C11 scFv IT-EP (FIG. 20). An increase antigen specific (SIINFEKL+) CD8+ T cells in TILs was observed in B16-OVA tumor model mice treated with 2C11 scFv IT-EP FIG. 21. The results demonstrate that IT-EP with 2C11 results in proliferation of polyclonal T cells and enhanced tumor specific T cell response in the tumor.

Example 12. In vivo cytotoxic T cells killing assay. Lymphocytes were harvested from naïve mice and labeled with CFSE. Label lymphocytes were then either pulsed with OVA peptide to activate T cells (CFSE^{hi}, treated) or left untreated (CFSE^{lo}, unpulsed). CFSE^{hi} and CFSE^{lo} lymphocytes were combined in an about 1:1 ratio for administration into the tumor bearing mice.

On Day −7, mice were implanted with B16-OVA tumor cells (B16 melanoma cells expressing ovalbumin) into the flank of c57/b1/6 mice. On Day 1, mice were treated with IT-EP anti-2C11 scFv or empty vector (pUMVC3). On day 2, mice administered pulsed target cells (cells pulsed with 2 μg/ml SIINFEKL peptide labeled with 1 μM CFSE (5(6)-carboxyfluorescein N-hydroxysuccinimidyl ester) and unpulsed cells by adoptive transfer. 18 hours after adoptive transfer, spleen and draining lymph nodes were collected and analyzed.

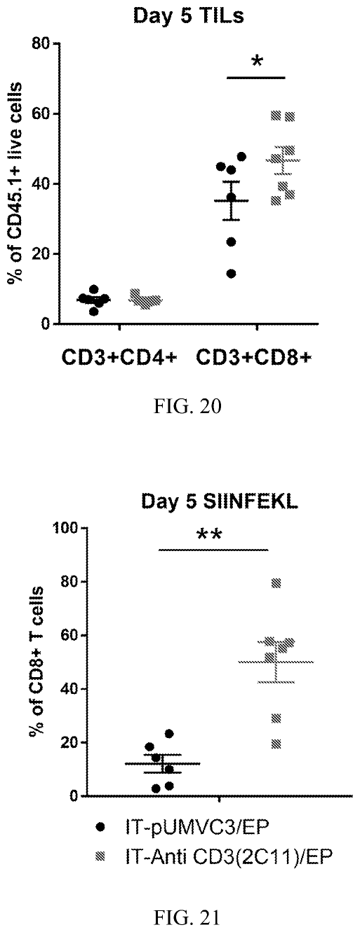

Western blot analysis indicated the tumors expressed the CD3 half-BiTE. On day 3, 18 h after adoptive transfer, DLN were isolated. DLNs were then analyzed by FACS for the presence of CFSE^{lo} and CFSE^{hi} cells. The results, shown in FIG. 22, show a substantial decrease in the number of CFSE^{hi} cells, indicated antigen-specific killing of cells displaying the OVA peptide. The decrease was quantitated using the following formula:

$$\% lysis = 1 - \frac{[ pulsed / unpulsed of treated ]}{[ pulsed / unpulsed of naïve ]}$$

$$\% lysis = 1 - \frac{[ pulsed ( {CFSE}^{h i} ) EP anti - CD 3 \div unpulsed ( {CFSE}^{l o} ) EP anti - CD 3 ]}{[ pulsed ( {CFSE}^{h i} ) EP contol \div unpulsed ( {CFSE}^{l o} ) EP control ]}$$

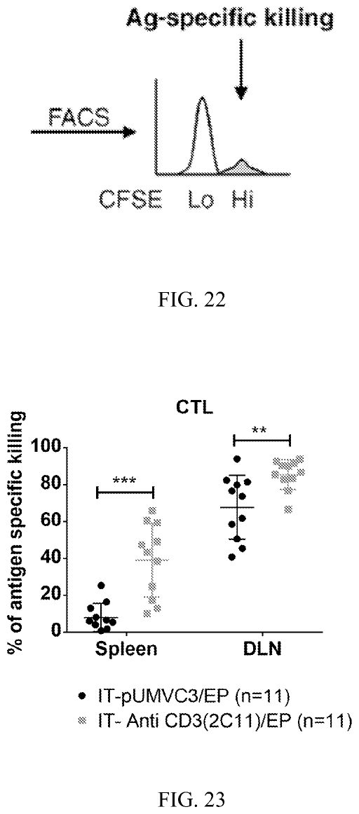

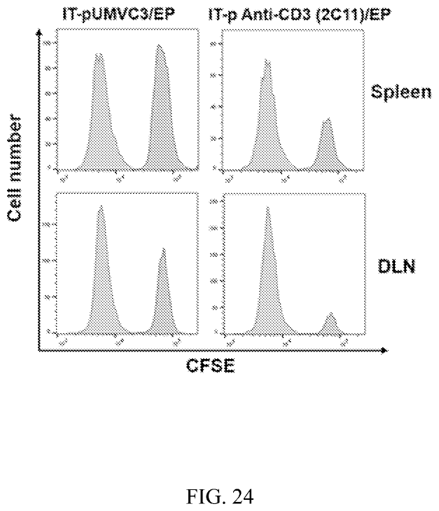

Results are shown in FIG. 23, showing an increase in lysis of CFSE^{hi} cells in both splenocytes (SP) and DLN. FACS analysis of CFSE cells is shown in FIG. 24. In control mice, percent lysis of CFSE^{hi} cells was 54.63±12.79%. In mice receiving IT-EP CD3 half-BiTE therapy, percent lysis of CFSE^{hi} cells was 82.44±11.35%. OVA expressing cells were specifically killed in mice treated with IT-EP CD3 half-BiTE, indicating the enhancement of an antigen-specific cytotoxic T cell response. Activated T lymphocytes were preferentially retained in tumors expressing a CD3 half-BiTE. Thus, electroporation of nucleic acid encoding a CD3 half-BiTE provides an effective tumor therapy.

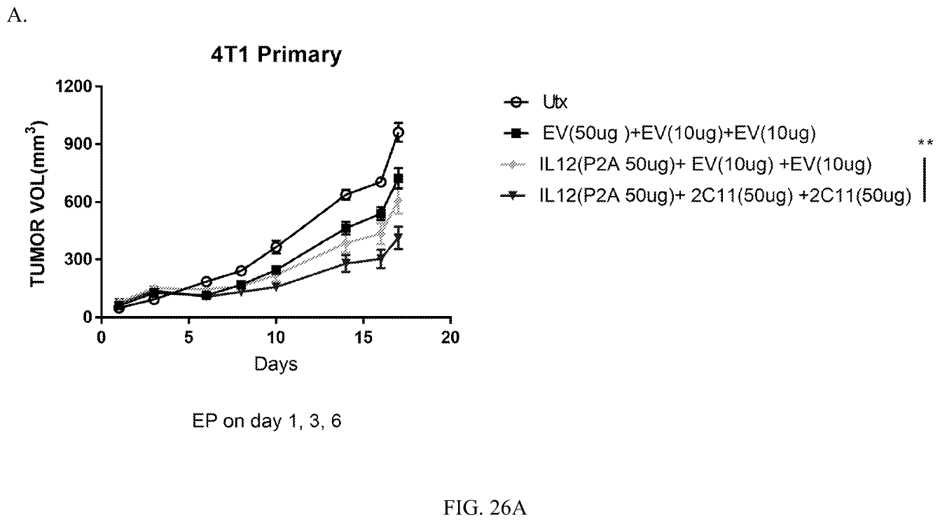

IT-EP of CD3 half-BiTE resulted in increased targeting of tumor cells by T cells. Flow cytometric analysis of cells from spleen and draining lymph node demonstrating significant antigen specific killing in the IT-EP anti-CD3(2C11) group (FIGS. 23 and 26).

### Example 13. Tumor Regression

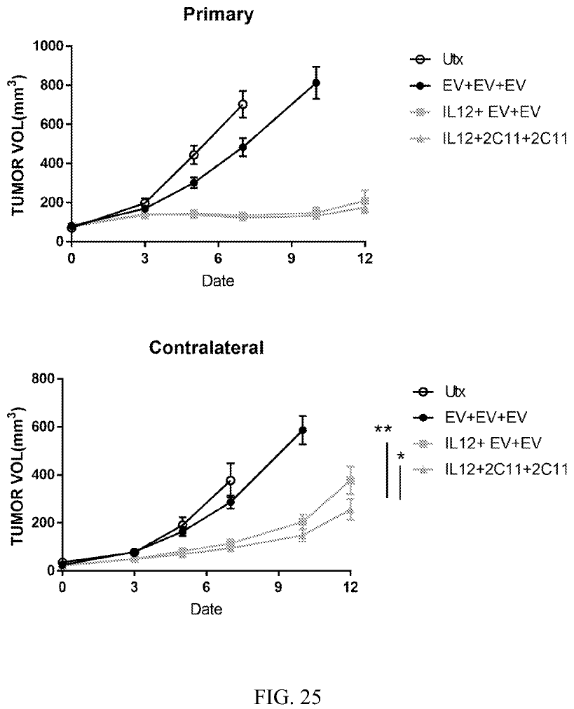

A. Melanoma: On Day −7, mice were implanted with B16 melanoma cells. On Day 0, mice were treated with IT-EP with control empty vector, expression vector encoding IL12-2A. On days 4 and 7, mice were treated with IT-EP control vector or IT-EP 2C11 (CD3 half-BiTE) expression vector. Tumor progression was monitored every three days. The results show improved contralateral (untreated) tumor regression in mice treated with IL12-2A plus CD3 half-BiTE compared to treatment with IL12-2A alone (FIGS. 25A and 25B).

B. Breast Cancer: On Day −7, mice were implanted with 4T1 breast cancer cells. On Day 0, mice were treated with IT-EP with control vector, or IT-EP IL12-2A. On days 4 and 7, mice were treated with IT-EP with control vector or IT-EP 2C11 (CD3 half-BiTE) expression vector. Tumor progression was monitored every three days. The results show that combining IT-EP IL12-2A with CD3 half-BiTE therapy improves breast cancer tumor regression (FIG. 26A). IL12-2A plus CD3 half-BiTE therapy was also effective in treating lung metastases nodules in 4T1 breast cancer model mice (FIG. 26B). The absolute number of effector T cells (CD127-CD62L-CD3⁺) per μL peripheral blood in 4T1 breast cancer model mice is shown in FIG. 26C.

CXCL9/CD3 half-BiTE Combination Therapy

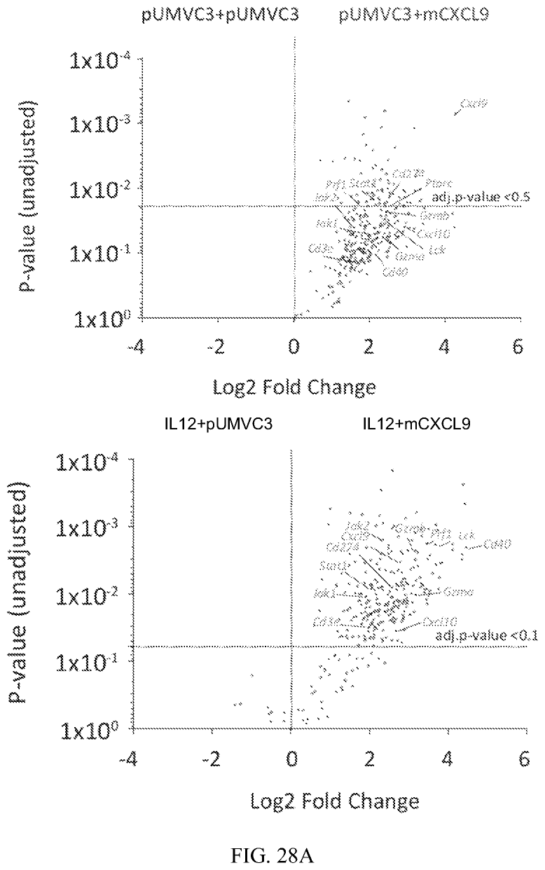

Example 14. CXCL9 plus CD3 half-BiTE combination therapy. B16.F10 tumor bearing mice were treated with IT-EP (days 1, 5, and 8) with 10 μg IL-12 expression plasmid, 100 μg IL-12 expression plasmid, or 100 μg IL-12˜CXCL9/CD3 half-BiTE˜IL12. For IL-12˜CXCL9/CD3 half-BiTE˜IL12, either IL-12˜CXCL9 or CD3 half-BiTE˜IL12 is administered on each of days 1, 5, and 8 provided the subject receives at least one IT-EP treatment with IL-12˜CXCL9 and one IT-EP treatment with CD3 half-BiTE˜IL12. Intratumoral expression of IL-12 was confirmed (ELISA) 48 hr post IT-EP in tumor lysates (n=8 animals). IL12p70 expression is shown in FIG. 29A. Growth of primary (electroporated lesion) and contralateral (non-electroporated lesion) B16.F10 lesions was measured 12 days after IT-EP therapy (FIG. 29B-C). With respect to IL12p70 expression, animal treated with IT-EP with 10 μg IL12-2A expressed the same amount of IL12 as animals treated with 100 μg IL-12˜CXCL9/CD3 half-BiTE˜IL12 (FIG. 29A). Contralateral tumors were significantly smaller in IL-12˜CXCL9/CD3 half-BiTE˜IL12 treated animals compare to 10 μg IL12-2A treated mice (8-10 animals/group; statistical significance determined using two way ANOVA * p<0.05), illustrating enhancement of tumor regression using IT-EP IL-12˜CXCL9/CD3 half-BiTE˜IL12 therapy.

CLTA-4 scFv

Example 15. Intratumoral expression of anti-CTLA4 scFv. Mouse IgG1 ELISA (ab133045) was performed on RENCA tumor lysates to quantify intratumoral expression of anti-CTLA4 scFv. Expression of anti-CTLA4 scFv was detected only in the tumor and not in the serum highlighting local expression of the antibody upon intratumoral electroporation.

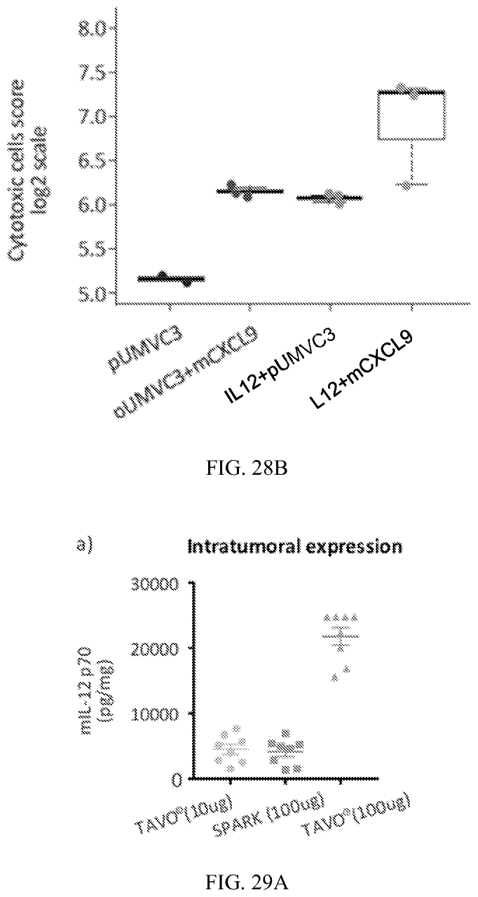

Plasmid encoded anti-CTLA4 scFv bound to recombinant CTLA4 protein. Transfection-derived secreted anti-CTLA4 (scFv) were evaluated for their binding capacity to CTLA-4. Recombinant mouse CTLA-4/human IgG1 chimera (R&D Systems) were immobilized in 96-well plates (1 or 5 μg/mL, or 50 μg/well or 250 μg/well) for 18 hr at room temperature. Wells were washed three times with 0.1% Tween in PBS and blocked with 1% BSA in PBS. Conditioned medium from HEK293 cells transfected with 9H10-scFv (168 ng/mL) or 9D9-scFv (130 ng/mL) was added to the wells, and incubated for 2 h at room temperature. Wells were washed three times, and anti-mouse IgG-horseradish peroxidase (Jackson ImmunoResearch, 0.2 μg/mL) were added and incubated for 1.5 hours at room temperature. Wells were again washed three times, developed with HRP Substrate Reagent (R&D Systems) and stopped with Stop Solution, 2N sulfuric acid (R&D Systems). Optical density of each well was measured at 450 nm. Graphical representation of average OD values for each condition group are displayed demonstrating binding of plasmid derived anti CTLA4scFv to recombinant CTLA4 protein (FIG. 30A).

Mouse IgG1 ELISA (ab133045) was performed on RENCA tumor lysates to quantify intratumoral expression of anti-CTLA4 scFv. Expression of anti-CTLA4 scFv was detected in the tumor (FIG. 30B). Statistically significant levels of anti-CTLA4 scFv was not observed in serum, indicating local expression of the antibody upon intratumoral electroporation.

It will be understood that the present invention has been described above by way of example only. The examples are not intended to limit the scope of the invention. Various modifications and embodiments can be made without departing from the scope and spirit of the invention, which is defined by the following claims only.

### CXCR3 Expression

Example 16. Intratumoral levels of CXCR3 are predictive of response to anti-PD-1/anti-PD-L1 therapy. Clinical and preclinical studies have demonstrated that plasmid IL-12 delivered intratumorally via electroporation drives IFN-γ expression and results in expansion of T cells and recruits T cells to the tumor microenvironment. This recruitment yields durable systemic T cell responses. Analysis of biomarker data from patients receiving IL-12/anti-PD-1 combination therapy shows that clinical response is correlated with intratumoral CXCR3 levels. While all patients had a similar frequency of activated CD8+ T cells in the periphery, responding patients had a significant increase of intratumoral CXCR3 transcripts post-treatment (p=0.03) compared to nonresponding patients (p=0.4). The presence of tumor-infiltrating CXCR3⁺ immune cells can therefore be used a biomarker for clinical response.

Intratumoral electroporation of CXCL9 leads to efficient trafficking of CXCR3⁺ CD8⁺ T cells into tumors. CXCL9 gradient can productively modulate frequencies of tumor infiltrating tumor-reactive CXCR3⁺ T cells.

As shown above, intratumoral electroporation of plasmid IL-12 and CXCL9 elicits a robust anti-tumor immune response evidenced by increased systemic antigen-specific CD8+ T cells and improved regression of both treated and contralateral tumors.

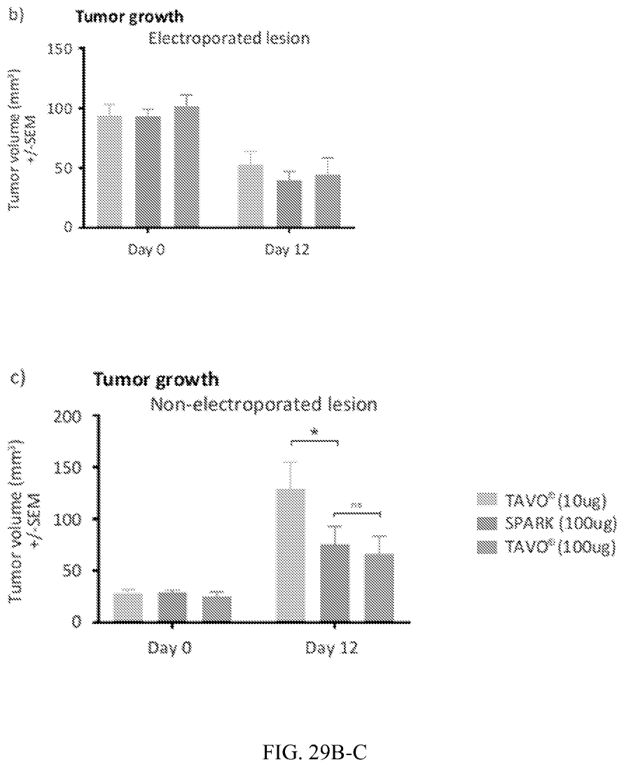

Example 17. Clinical response to IL-12 IT-EP and pembrolizumab combination therapy. Patients were treated with 0.5 mg/ml of IT-EP IL-12 to accessible lesions on days 1, 5, and 8 every 6 weeks, and 200 mg IV pembrolizumab on day 1 of each 3-week cycle for 27 weeks (FIG. 31). Nucleic acid encoding IL-12 was injected at a dose volume of about ¼ of the calculated lesion volume, with a minimum dose volume of 0.1 mL. Electroporation was administered using an applicator having a hexagonal arrow of 6 microneedles. The microneedles were placed into and around the injected tumor, with the tip co-localized at the site(s) and depth of the plasmid injection. Six pulses at a field strength of 1500 V/cm and a pulse width of 100 μsec at 300 millisec intervals were delivered.

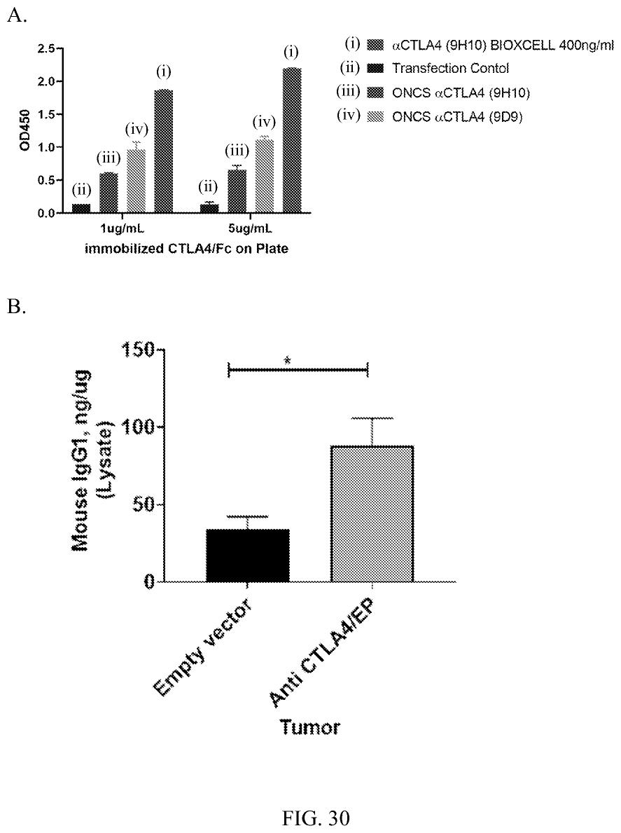

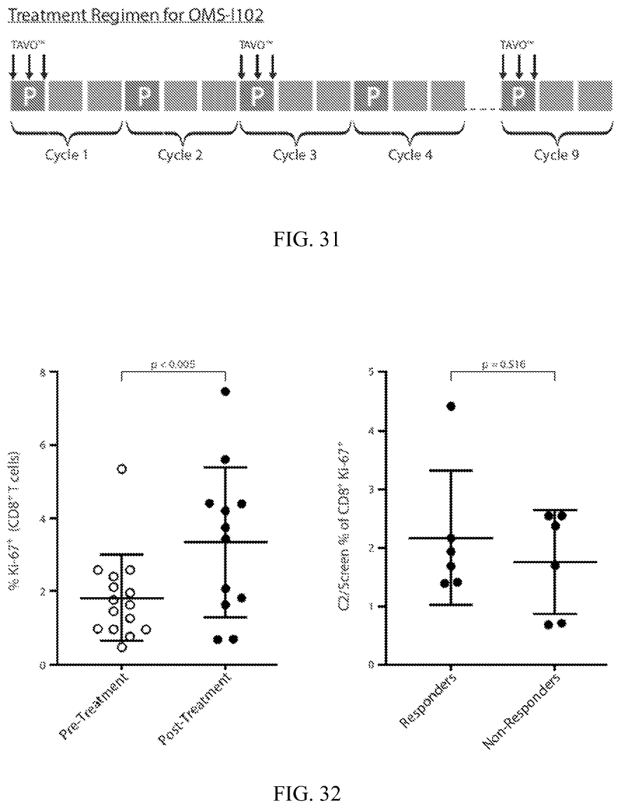

An increase in proliferating Ki-67⁺ CD8⁺ T cells post treatment was observed in both responders and non-responders. Ki-67⁺ CD8+ T cells were analyzed by flow cytometry (gating strategy Singlets<Live<CD3<CD8) prior to treatment (pre-treatment) and after treatment cycle 2 (post-treatment) in both responding and non-responding patients (n=6 per group) (FIG. 32). In contrast, an increase in intratumoral CXCR3 transcript levels was observed only in responders (n=5 for responders and n=8 for non-responders) (FIG. 33).

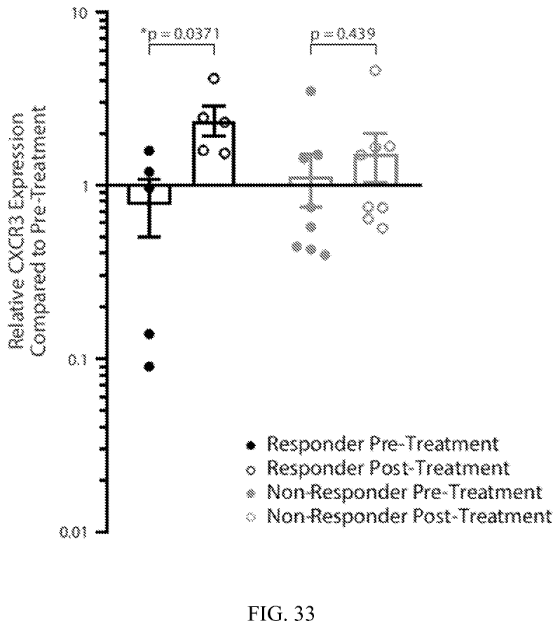

Example 18. Anti-CXCR3 antibodies inhibit tumor regression mediated by pIL12 IT-EP therapy. IT-EP IL-12 (TAVO(P2A)) resulted in an increase in CXCR3⁺ lymphocytes in the draining lymph node. CT26 tumors were implanted into the flanks of mice. After development of tumors, mice were treated IT-EP with either 50 μg IL-12 (TAVO(P2A)) or 50 μg control (empty) vector (EV). After 24 hours, PBMCs were obtained from the mice and analyzed for CD8+ CXCR3+ T cells by flow cytometry (gating strategy Live<singlets<CD45⁺CD3⁺<CD8⁺CD4⁻, MFI=mean fluorescence intensity) (FIG. 34).

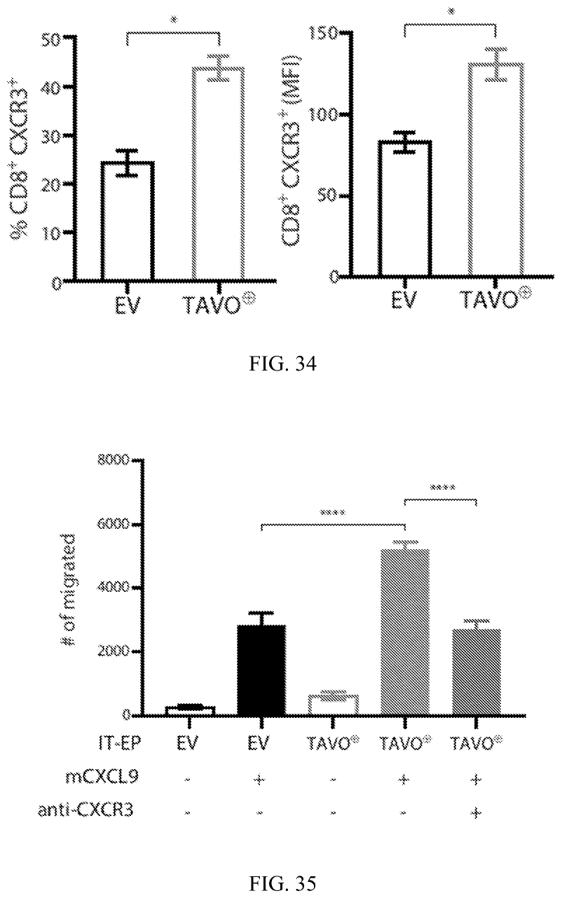

After 96 hours, cells were obtained from draining lymph nodes (DLN) and analyzed for CXCL9-induced chemotaxis (FIG. 35). Cells from DLN were placed in an upper chamber separated from a lower chamber by a polycarbonate membrane having 5.0 micron pores (Costar 3421). The lower chamber contained conditioned media from HEK cells transfected with a plasmid expressing CXCL9. The number of observed chemotactic cells was observed after 2 hours at 37° C. Cells from DLN of mice treated with IT-EP TAVO(P2A) showed increase migration compared to cells from DLN of mice treated with empty vector. Pre-incubation of cells with anti-CXCR3 monoclonal antibody (BioXCell BE0249) abrogated with chemotaxis, indicating the increase in chemotaxis was the result of increase CXC3R⁺ cells.

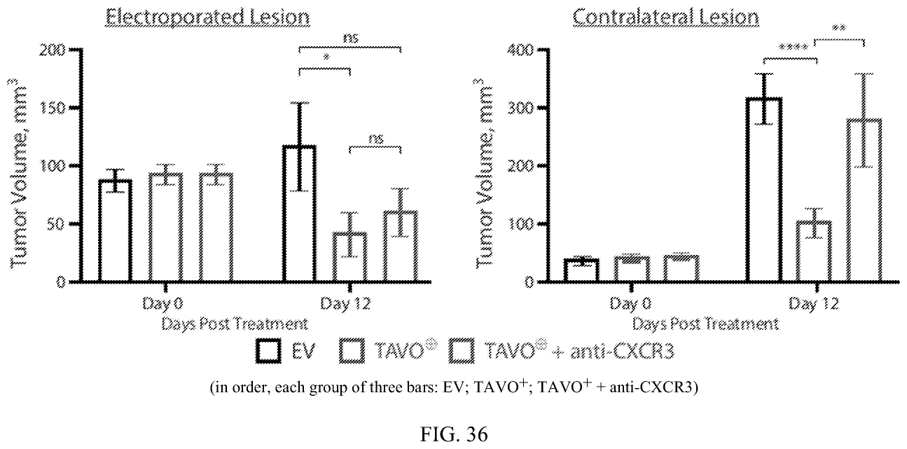

CXCR3 depletion resulted in complete abrogation of IL-12 response. CT26 contralateral tumor model was treated with IT-EP IL-12, with or without concomitant anti-CXCR3 antibody treatment. Growth of primary (electroporated) tumors and untreated (contralateral) tumors after IT-EP with empty vector, TAVO(P2A), or TAVO(P2A) plus anti-CXCR3 was analyzed. Contralateral tumor growth was inhibited in mice treated with IT-EP IL-12 (TAVO(P2A)). This inhibition was blocked by anti-CXCR3 antibodies (FIG. 36).

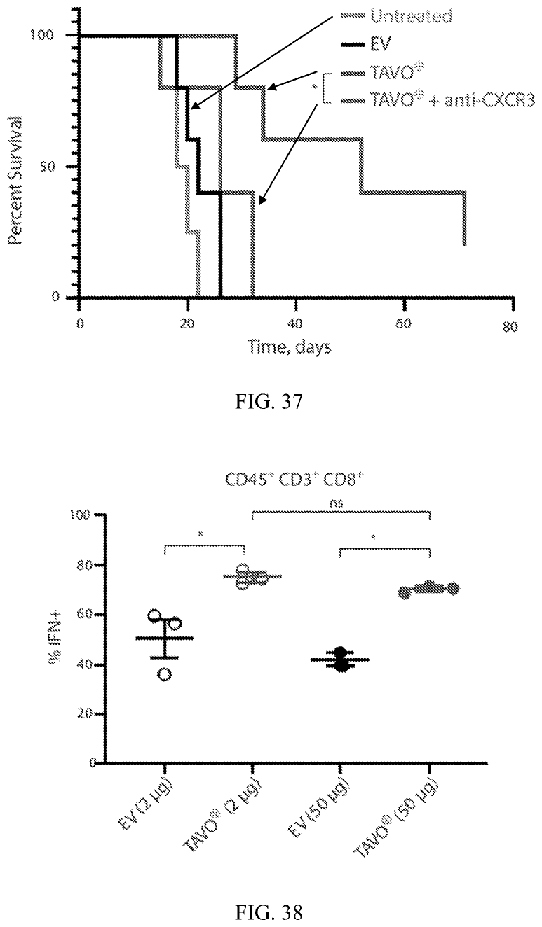

Further, mice treated with IT-EP IL-12 showed a significant survival benefit. The survival benefit was also blocked by anti-CXCR3 antibodies (FIG. 37).

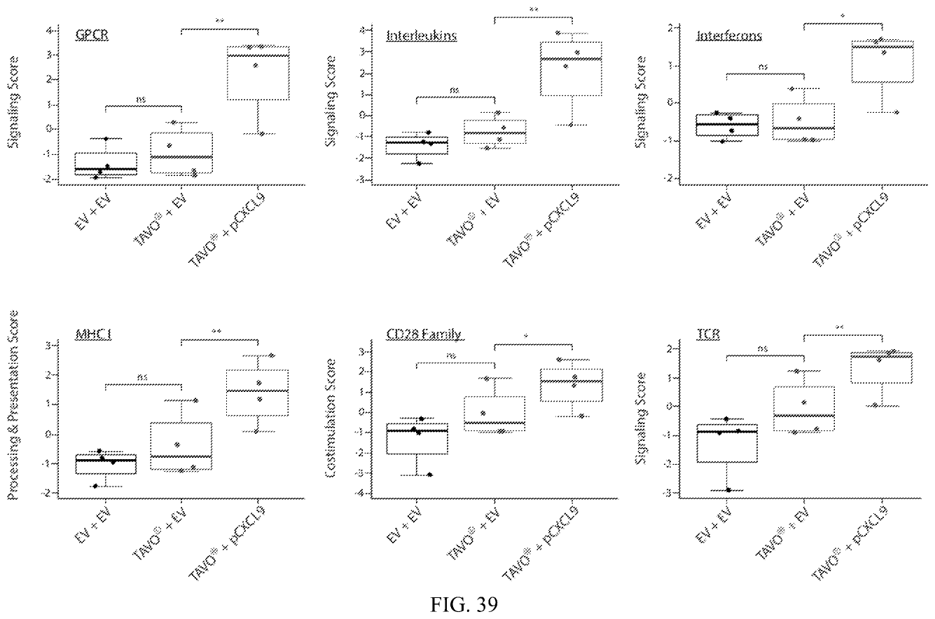

Example 19. Intratumoral electroporation of IL-12 plus CXCL9 enhanced anti-tumor efficacy of IT-EP IL-12. CT26 contralateral tumor mouse models were treated with IT-EP IL-12, using either 2 μg (low dose) or 50 μg empty vector or IL-12 (TAVO(P2A)). After 24 hours, tumor resident CD8⁺ T cells were isolated and stained for intracellular IFN-γ expression. Intratumor IFN-γ expression by CD8⁺ T cells was similar regardless of the IL-12 dose, indicating that low dose (2 μg) IL-12 is immunologically active (FIG. 38).

Mice were next treated with IT-EP empty vector, IT-EP IL-12 (TAVO(P2A)) plus IT-EP empty vector, or IT-EP IL-12 (TAVO(P2A)) plus IT-EP CXCL9 (plasmid expression CXCL9) as indicated in Table 2.

TABLE 2

| Group | Day 1 | Day 5 | Day 8 |
|---|---|---|---|
| 1 | 2 μg EV | 100 μg EV | 100 μg EV |
| 2 | 2 μg TAVO(P2A) | 100 μg EV | 100 μg EV |
| 3 | 2 μg TAVO(P2A) | 100 μg CXCL9 | 100 μg CXCL9 |

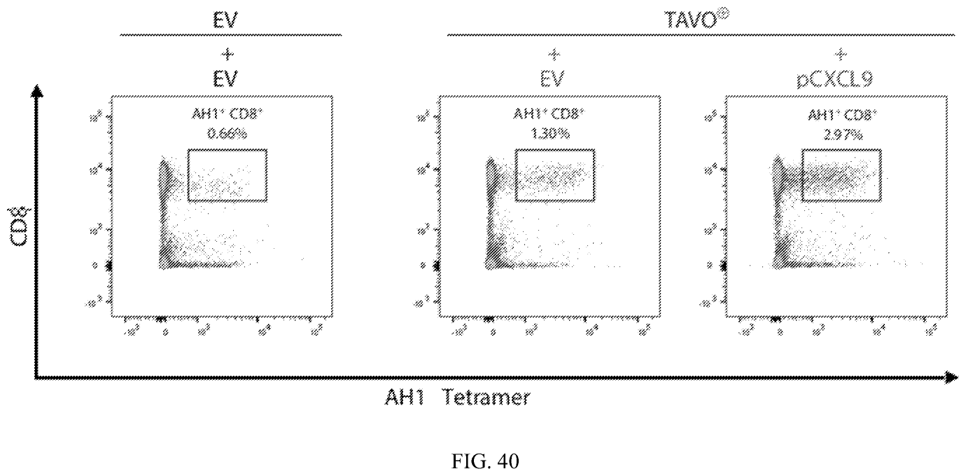

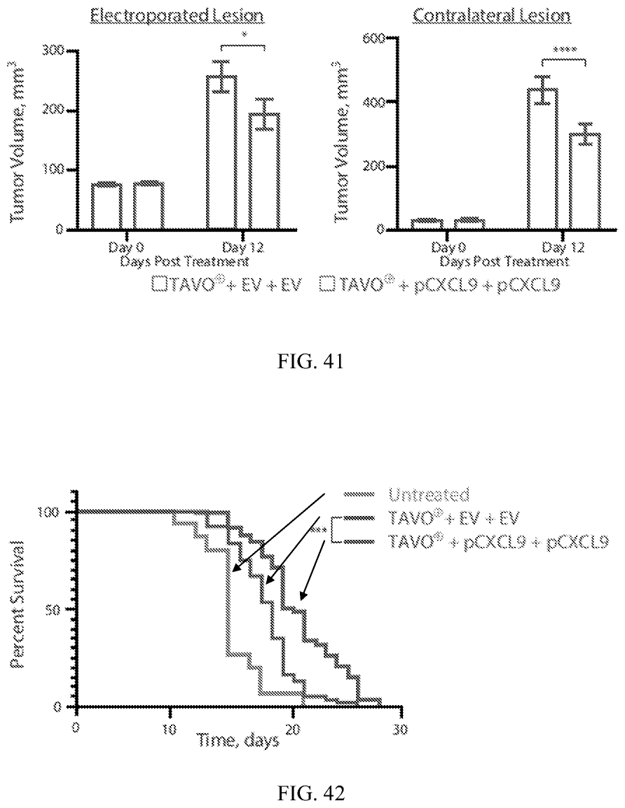

Spleens and treated tumors were harvested at day 10 and analyzed by NanoString and flow cytometry. Gene expression changes in electroporated tumors were analyzed by NanoString nCounter technology (mouse PanCancer io360 panel) with pathway scores. Pathway scores followed the assumptions of equal variance and normal distribution of t scores. Ordinary one-way ANOVA was used to calculate significance compared to the empty vector treated group (n=4 per group). Transcriptomic analysis of the tumor microenvironment revealed an enrichment of genes associated with immune-related pathways (IFNγ signaling, interleukin signaling, GPCR signaling), antigen presentation machinery, and TCR signaling, indicating that this combination therapy augmented anti-tumor immunity (FIG. 39).

Antigen specific CD8 T cells (AH1⁺CD8⁺) were enriched in mice treated with IT-EP IL-12 plus IT-EP CXCL9 when compared to IT-EP IL-12 alone or empty vector (gating strategy SSC<Live<Singlets<CD4⁻ splenocytes).

A significant increase in the percentage of AH1⁺CD8⁺ T cells was observed in IT-EP IL-12 treated and IT-EP IL-12 plus CXCL9 treated mice compared to empty vector control treated mice.

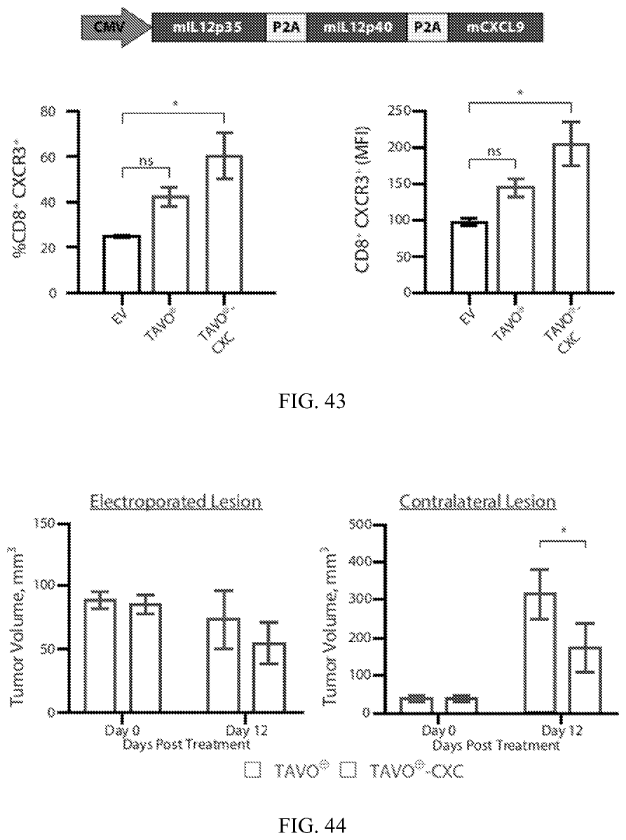

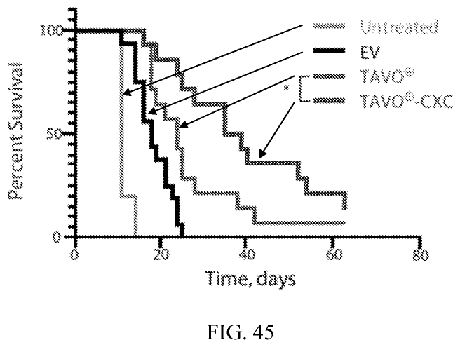

Example 20. IT-EP CXCL9 augments IT-EP IL-12 mediated abscopal anti-tumor response. Mice bearing implanted tumors in both the left and right flank (contralateral tumor model) were treated as in Table 2. Treatment was administered to only one tumor. Tumor volumes were measured three times per week for regression and survival studies. Growth of treated (left panel) and untreated tumors showed that sequential treatment with IT-EP IL-12 therapy and IT-EP CXCL9 therapy resulted in improved tumor regression and improved survival in both treated and untreated (contralateral) tumors (FIG. 41).

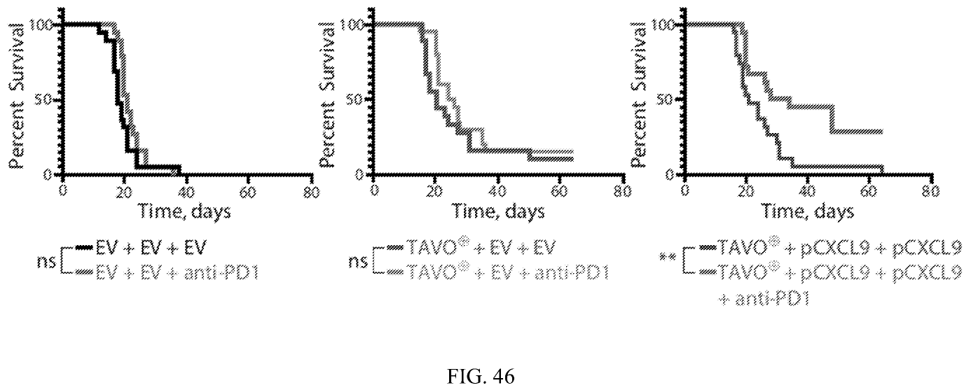

Mice were treated with IT-EP IL-12 plus CXCL9 using a plasmid expressing IL-12 p35, IL-12 p40, and CXCL9 from a single CMV promoter. Frequency and mean fluorescence intensity of CXCR3⁺ expression of CD8 cells from tumors collected 24 hours post IT-EP treatment with empty vector, TAVO(P2A), or TAVO(P2A)-CXCL9 showed an increase in CD8⁺ CXCR3⁺ cells after IT-EP of IL-12 or IL-12 plus CXCL9 (FIG. 43).

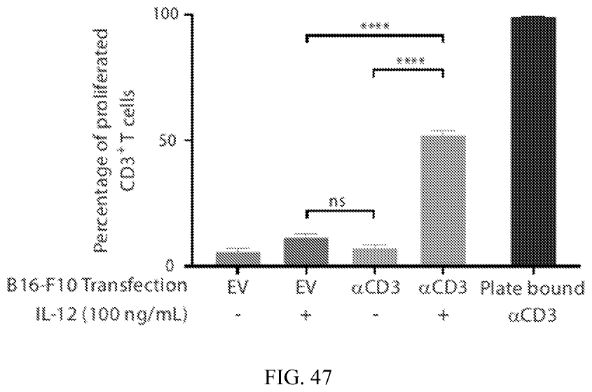

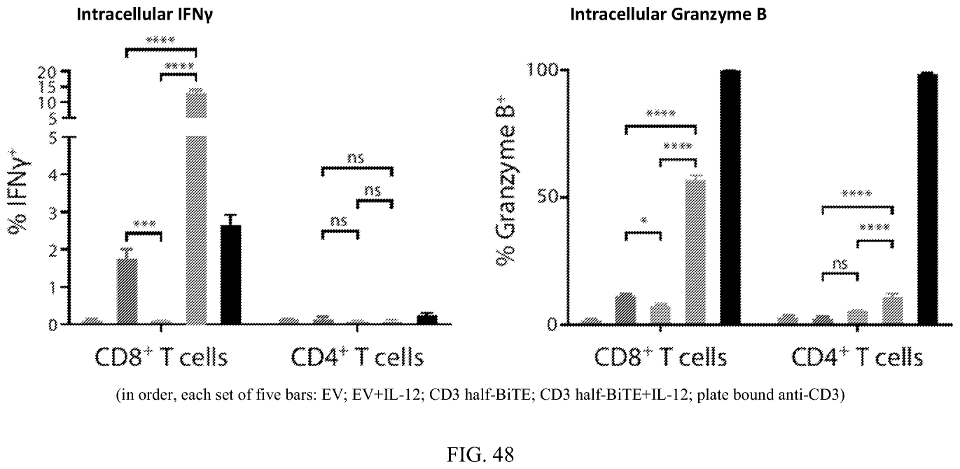

CT26 tumors cells were implanted in the left and right flank of mice on day −7. Tumor-implanted mice were then treated on days 1, 5, and 8 with either IT-EP empty vector (group 1), IT-EP IL-12 (TAVO(P2A); group 2), or IT-EP IL-12-CXCL9 (TAVO(P2A)-CXCL9; group 3) in one of the tumors. The plasmid dose was normalized to the amount of IL-12 produced in mice treated with TAVO(P2A)-CXCL9 plasmid, as determined by ELISA. At 12 days post-treatment, contralateral tumors from the IT-EP IL-12-CXCL9 group were significantly smaller (FIG. 44). Mice treated with IT-EP IL-12˜CXCL9 also exhibited a survival advantage (FIG. 45).

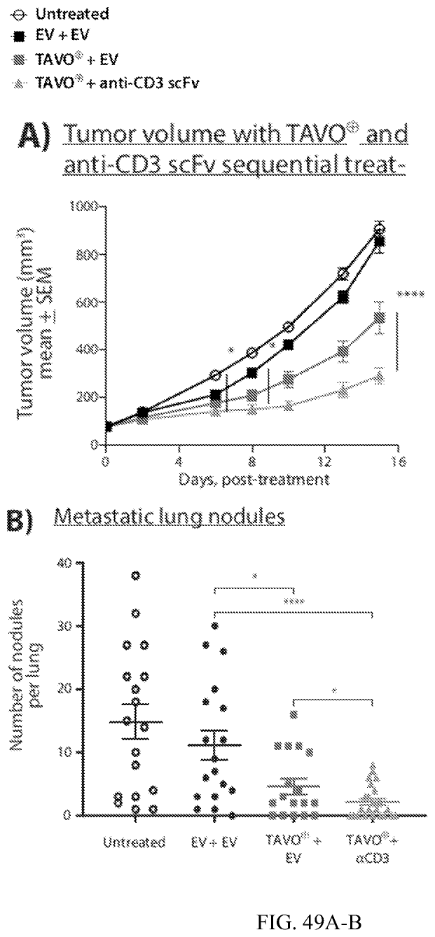

Example 21. Intratumoral electroporation of IL-12 and CXCL9 improves anti-PD-1 therapy. CT-26 tumor cells were implanted into the left and right flank of mice. Mice were then treated according to the schedule in Table 3. Tumor volumes were measured three times per week for regression and survival studies (FIG. 46).

TABLE 3

| | Day 1 | | Day 5 | Day 8 | | Day 15 |
|---|---|---|---|---|---|---|
| | IP | IT-EP | IT-EP | IP | IT-EP | IP |
| Group | 150 μg | 2 μg | 100 μg | 150 μg | 100 μg | 150 μg |
| 1a | control | EV | EV | control | EV | control |
| 1b | anti-PD-1 | EV | EV | anti-PD-1 | EV | anti-PD-1 |
| 2a | control | TAVO(P2A) | EV | control | EV | control |
| 2b | anti-PD-1 | TAVO(P2A) | EV | anti-PD-1 | EV | anti-PD-1 |
| 3a | control | TAVO(P2A) | CXCL9 | control | CXCL9 | control |
| 3b | anti-PD-1 | TAVO(P2A) | CXCL9 | anti-PD-1 | CXCL9 | anti-PD-1 |

IT-EP CXCL9, when combined with IT-EP IL12, caused increased generation of antigen-specific CD8⁺ cytotoxic T lymphocytes (AH1⁺ CD8⁺), enhanced abscopal response to IL-12 therapy, and improved anti-PD-1 antitumor response. Without wishing to be bound by theory, IL-12 may increase the activation and/or proliferation of CXCR3⁺ T cells. CXCR3⁺ T cells are then recruited to the tumor by the CXCR3 chemokine activity of CXCL9.

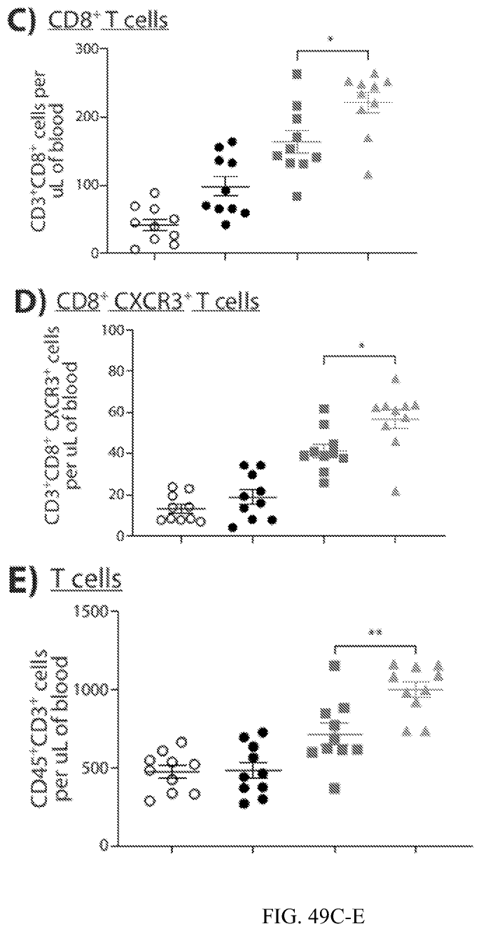

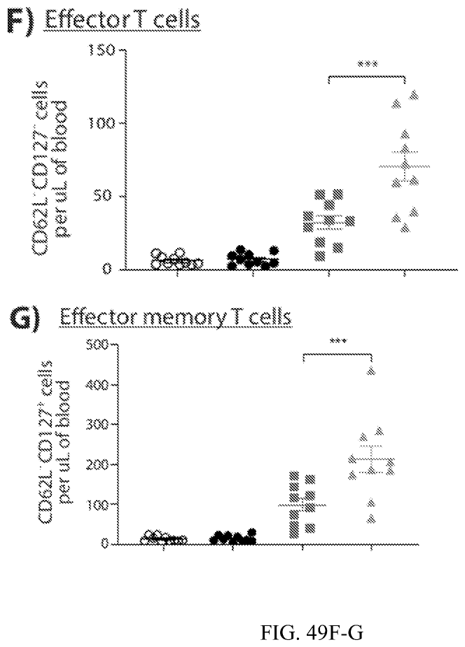

Example 22. IT-EP CD3 half-BiTE increases CXCR3⁺ T cells in tumors. Biomarker data have identified non-tumor reactive TILs that, if mobilized could increase clinical immunotherapy response. CD3 half-BiTE expression on neoplastic and stromal cells activates CD3⁺ TILs, driving enhanced proliferation and cytotoxicity. Naïve T cells, Treg cells, and exhausted T cells (subsets not typically associated with strong anti-tumor responses) displayed enhanced effector function (IFNγ and granzyme B release) with engagement of CD3 half-BiTE and IL-12 (FIG. 47-48).

The combination of IL-12 and CD3 half-BiTE enhanced proliferation of T cells regardless of the affinity for their cognate peptide:MHC, suggesting a T cell receptor independent mechanism. Thus, IT-EP IL-12 plus CD3 half-BiTE can mobilize broad subsets of T cells.

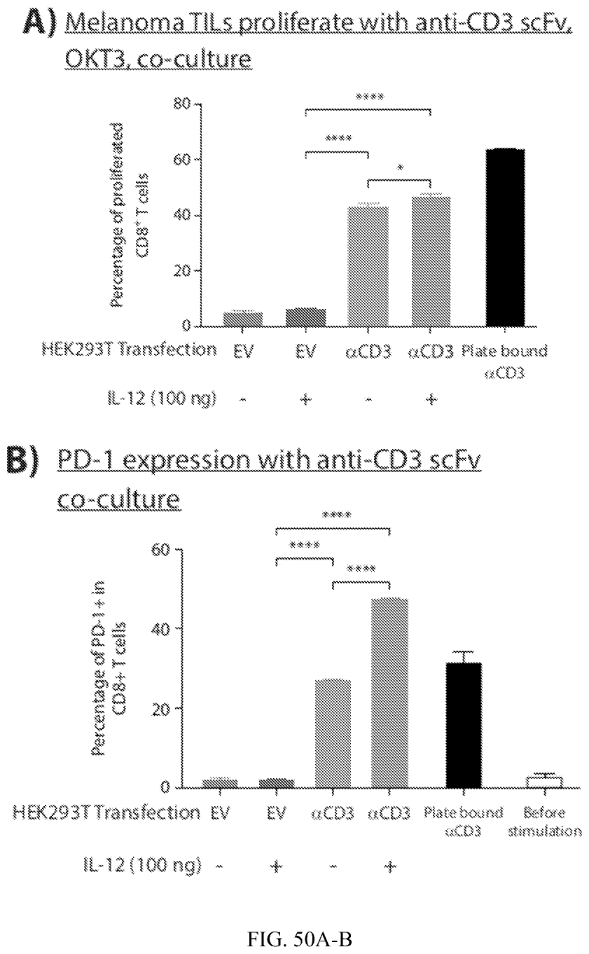

Using immune profiling of the tumor microenvironment (TME), it was found that IT-EP CD3 half-BiTE significantly upregulated frequencies of CXCR3⁺ CD8⁺ T cells and short-lived effector T cells. We further show that functional restoration of TILs in melanoma patients with active clinical progression on anti-PD-1 therapy was possible with engagement of CD3 half-BiTE in the presence of IL-12 (FIG. 49).

4T1 tumors were treated with IT-EP using 50 μg of empty vector (EV) or IL-12 (TAVO(P2A)) on Day 0 followed by subsequent IT-EP on days 3 and 5 with 50 μg of EV or CD3 half-BiTE: (A) tumor volume, (B) spontaneous metastatic lung modules, (C) CD3⁺ CD8⁺ T cells, (D) CD8⁺ CXCR3⁺ T cells, (E) CD45⁺ CD3⁺ T cells, (F) effector T cells, and (G) effector memory T cells. T cell populations were measured 6 days post IT-EP treatment.

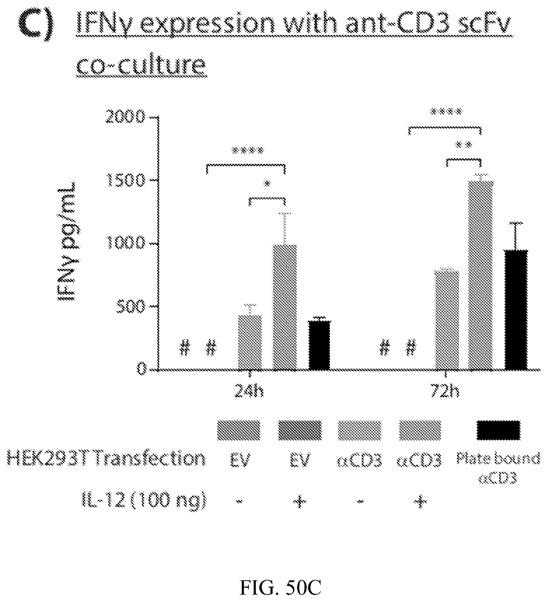

TILs isolated from patients actively progressing on anti-PD-1 therapy were co-cultured for three days with HEK293T cells transfected with empty vector or CD3 half-BiTE with or without IL-12. TILs cultured with plate bound anti-human CD3 were used as a positive control. The percentage or CD8+ T cells, the percentage of PD-1+ CD8+ T cells, and the level of IFNγ expression was higher in TILs incubated with HEK293T cells transfected with CD3 half-BiTE, indicating that TILs from patient progressing on anti-PD-1 therapy recover immune function in response to CD3 half-BiTE (FIG. 50).

IT-EP CD3 half-BiTE or IT-EP IL-12˜CD3 half-BiTE can increase numbers of effector T cells, effector memory T cells, and activated T cells in peripheral blood, increase antigen-specific cytotoxicity, decrease metastatic tumor burden, and restore functional activity in TILs in patients progressing on checkpoint inhibitor therapy.

1. A method of treating cancer in a subject, the method comprising:
(a) measuring CXCR3 expression in the tumor sample obtained from the subject that has been previously treated with at least one dose of a checkpoint inhibitor and/or at least one dose of an immunostimulatory cytokine;
(b) determining whether CXCR3 expression is increased in the tumor sample relative to CXCR3 expression in a predetermined control; and
(c) if CXCR3 expression is increased in the tumor sample relative to CXCR3 expression in the predetermined control, then administering at least one additional dose of the checkpoint inhibitor and/or at least one additional dose of the immunostimulatory cytokine to the subject, or if CXCR3 expression is not increased in the tumor sample relative to CXCR3 expression in the predetermined control, then administering at least one pharmaceutically effective dose of CXCL9 and/or CD3 half-BiTE and at least one additional dose of the checkpoint inhibitor and/or at least one additional dose of the immunostimulatory cytokine to the subject.

2. The method of claim 1, wherein the subject has been previously treated with systemic administration of the at least one dose of the checkpoint inhibitor wherein the checkpoint inhibitor.

3. The method of claim 2, wherein the checkpoint inhibitor comprises an anti-PD-1 antibody, an anti-PD-L1 antibody, nivolumab, pembrolizumab, pidilizumab, or atezolizumab.

4. The method of claim 1, wherein the subject has been previously treated with the at least one dose of the immunostimulatory cytokine wherein the immunostimulatory cytokine was administered by intratumoral electroporation of a nucleic acid encoding the immunostimulatory cytokine.

5. The method of claim 4, wherein the immunostimulatory cytokine comprises IL-12 or IL-15.

6. The method of claim 5, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

7. The method of any one of claims 1-6, wherein measuring CXCR3 expression in the tumor sample comprises:
(a) measuring CXCR3 mRNA in the tumor sample;
(b) measuring CXCR3 protein in the tumor sample; or
(c) measuring a number of CXCR3⁺ T cells in the tumor sample.

8. The method of claim 7, wherein the predetermined control comprises:
(a) a tumor sample obtained from the subject prior to step (a); or
(b) a standard derived from a population of known responders and/or known non-responders to checkpoint inhibitor and/or an immunostimulatory cytokine therapy.

9. The method of any one of claims 1-8, wherein administering at least one pharmaceutically effective dose of CXCL9 and/or CD3 half-BiTE comprises intratumoral electroporation of one or more nucleic acids encoding one or more of CXCL9, CD3 half-BiTE, CXCL9 and IL-12, or CD3 half-BiTE and IL-12.

10. The method of claim 1, wherein administering at least one additional dose of the checkpoint inhibitor and/or the least one additional dose of the immunostimulatory cytokine comprises: administering the at least one additional dose of an anti-PD-1 or anti-PD-L1 antibody by systemic administration, administering at least one additional dose of a nucleic acid encoding IL-12 by intratumoral electroporation, or administering of at least one additional dose of an anti-PD-1 or anti-PD-L1 antibody by systemic administration and administering at least one additional dose of a nucleic acid encoding IL-12 by intratumoral electroporation.

11. The method of claim 10, wherein the nucleic acid encoding the IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

12. The method of any one of claims 1-11, wherein the cancer is melanoma, basal cell carcinoma, breast cancer, ER positive breast cancer, ER negative breast cancer triple negative breast cancer, or head and neck cancer.

13. A method of identifying a subject with cancer at risk of not responding to checkpoint inhibitor and/or immunostimulatory cytokine therapy, the method comprising:
measuring a level of CXCR3 in a tumor sample obtained from the subject that has been administered at least one dose of a checkpoint inhibitor and/or at least one dose of an immunostimulatory cytokine,
wherein the level of CXCR3 in the tumor sample less than a predetermined control indicates the subject is at risk of not responding to the checkpoint inhibitor and/or immunostimulatory cytokine therapy, and the level of CXCR3 in the tumor sample more than a predetermined control indicates the subject is likely to respond to the checkpoint inhibitor and/or immunostimulatory cytokine therapy.

14. The method of claim 13, wherein measuring the level of CXCR3 in the tumor sample comprises
(a) measuring CXCR3 mRNA in the tumor sample;
(b) measuring CXCR3 protein in the tumor sample; or
(c) measuring a number of CXCR3⁺ T cells in the tumor sample.

15. The method of claim 13 or 14, wherein the predetermined control comprises:
(a) a tumor sample obtained from the subject prior to the subject being administered the at least one pharmaceutically effective dose of the checkpoint inhibitor and/or the immunostimulatory cytokine; or
(b) a standard derived from a population of known responders and/or known non-responders to checkpoint inhibitor and/or an immunostimulatory cytokine therapy.

16. A nucleic acid encoding CXCL9 and/or CD3 half-BiTE for use in a method of treating cancer, the method comprising:
(a) measuring CXCR3 expression in a tumor sample obtained from the subject;
(b) determining whether CXCR3 expression is increased in the tumor sample relative to CXCR3 expression in a predetermined control; and
(c) administering at least one pharmaceutically effective dose of CXCL9 and/or CD3 half-BiTE and at least one pharmaceutically effective dose of a checkpoint inhibitor and/or at least one pharmaceutically effective dose of an immunostimulatory cytokine to the subject if CXCR3 expression is not increased in the tumor sample relative to CXCR3 expression in the predetermined control.

17. The nucleic acid for use in the method of claim 16, wherein measuring CXCR3 expression in the tumor sample comprises
(a) measuring CXCR3 mRNA in the tumor sample;
(b) measuring CXCR3 protein in the tumor sample; or
(c) measuring a number of CXCR3⁺ T cells in the tumor sample.

18. The nucleic acid for use in the method of claim 17, wherein the subject previously received at least one dose of a checkpoint inhibitor and/or at least one dose of an immunostimulatory cytokine prior to the tumor sample being obtained.

19. The nucleic acid for use in the method of claim 16, wherein the predetermined control comprises:
(a) a tumor sample obtained from the subject prior to the subject being administered the at least one dose of the checkpoint inhibitor and/or the at least one dose of the immunostimulatory cytokine; or
(b) a standard derived from a population of known responders and/or known non-responders to checkpoint inhibitor therapy and/or an immunostimulatory cytokine therapy.

20. The nucleic acid for use in the method claim 16, wherein the checkpoint inhibitor comprises an anti-PD-1 antibody, an anti-PD-L1 antibody, nivolumab, pembrolizumab, pidilizumab, or atezolizumab.

21. The nucleic acid for use in the method of claim 20, wherein the checkpoint inhibitor is administered systemically.

22. The nucleic acid for use in the method of claim 16, wherein administering at least one pharmaceutically effective dose of the immunostimulatory cytokine comprises administering a pharmaceutically effective dose of a nucleic acid encoding the immunostimulatory cytokine by intratumoral electroporation.

23. The nucleic acid for use in the method of claim 22, wherein the immunostimulatory cytokine comprises IL-12 or IL-15.

24. The nucleic acid for use in the method of claim 23, wherein the nucleic acid encoding IL-12 comprises a first nucleic acid sequence encoding an IL-12 p35 subunit and a second nucleic acid sequence encoding an IL-12 p40 subunit wherein the first and second nucleic acid sequences are separated by an internal ribosome entry site (IRES) or a 2A translation modification element.

25. The nucleic acid for use in the method of any one of claims 16-24, wherein administering at least one pharmaceutically effective dose of CXCL9 and/or CD3 half-BiTE comprises intratumoral electroporation of one or more nucleic acids encoding one or more of CXCL9, CD3 half-BiTE, CXCL9 and IL-12, or CD3 half-BiTE and IL-12.

26. The nucleic acid for use in the method of any one of claim 25, wherein the one or more nucleic acids encoding one or more of CXCL9, CD3 half-BiTE, CXCL9 and IL-12, and/or CD3 half-BiTE and IL-12 are administered on days 1, 5, and 8 of at least one three or six week cycle.

27. The nucleic acid for use in the method of claim 26, wherein the checkpoint inhibitor is administered on day 1 of at least one three week cycle.

28. The method of any one of claims 1-15 wherein administering at least one pharmaceutically effective dose of CXCL9 and/or CD-3 half-BiTE results in increased numbers of CXCR3⁺ T cells in the tumor.


SEQUENCE LISTING (89 sequences)

SEQ ID NO: 1 - DNA, 63 bp, Artificial Sequence
  IgKappa signal sequence
atggagacagacacactcctgctatgggtactgctgctctgggttccaggttccactggtgac

SEQ ID NO: 2 - protein, 21 aa, Artificial Sequence
  IgKappa signal sequence
METDTLLLWVLLLWVPGSTGD

SEQ ID NO: 3 - DNA, 27 bp, Artificial Sequence
  HA tag sequence
tatccatatgatgttccagattatgct

SEQ ID NO: 4 - protein, 9 aa, Artificial Sequence
  HA tag sequence
YPYDVPDYA

SEQ ID NO: 5 - DNA, 30 bp, Artificial Sequence
  Myc tag sequence
gaacaaaaactcatctcagaagaggatctg

SEQ ID NO: 6 - protein, 10 aa, Artificial Sequence
  Myc tag sequence
EQKLISEEDL

SEQ ID NO: 7 - DNA, 357 bp, Artificial Sequence
  2C11 Variable heavy chain sequence
gaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggcggt

SEQ ID NO: 8 - protein, 119 aa, Artificial Sequence
  2C11 Variable heavy chain sequence
EVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGG

SEQ ID NO: 9 - DNA, 312 bp, Artificial Sequence
  2C11 Variable light chain sequence
atgacccagtctccatcatcactgcctgcctccctgggagacagagtcactatcaattgtcaggccagtcaggacattagcaattatttaaactggtaccagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaa

SEQ ID NO: 10 - DNA, 312 bp, Artificial Sequence
  2C11 Variable light chain sequence
atgacccagtctccatcatcactgcctgcctccctgggagacagagtcactatcaattgtcaggccagtcaggacattagcaattatttaaactggtatcagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaa

SEQ ID NO: 11 - protein, 104 aa, Artificial Sequence
  2C11 Variable light chain sequence
MTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIK

SEQ ID NO: 12 - DNA, 45 bp, Artificial Sequence
  Linker sequence
agtggctctggagggggctctggcggtggatctgggggtggaagt

SEQ ID NO: 13 - protein, 15 aa, Artificial Sequence
  Linker sequence
SGSGGGSGGGSGGGS

SEQ ID NO: 14 - DNA, 45 bp, Artificial Sequence
  Linker sequence
ggtggcggtggctccggcggtggtgggtcgggtggcggcggatct

SEQ ID NO: 15 - protein, 15 aa, Artificial Sequence
  Linker sequence
GGGGSGGGGSGGGGS

SEQ ID NO: 16 - DNA, 36 bp, Artificial Sequence
  Linker sequence
ggctccggcggtggtgggtcgggtggcggcggatct

SEQ ID NO: 17 - protein, 11 aa, Artificial Sequence
  Linker sequence
GSGGGGSGGGG

SEQ ID NO: 18 - DNA, 27 bp, Artificial Sequence
  Linker sequence
ggcagtgggagtgggagtgggagtggg

SEQ ID NO: 19 - protein, 9 aa, Artificial Sequence
  Linker sequence
GSGSGSGSG

SEQ ID NO: 20 - DNA, 15 bp, Artificial Sequence
  Linker sequence
ggcagtgggagtggg

SEQ ID NO: 21 - protein, 5 aa, Artificial Sequence
  Linker sequence
GSGSG

SEQ ID NO: 22 - DNA, 15 bp, Artificial Sequence
  Linker sequence
tctagtggatccggt

SEQ ID NO: 23 - protein, 5 aa, Artificial Sequence
  Linker sequence
SSGSG

SEQ ID NO: 24 - DNA, 144 bp, Homo sapiens
gtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 25 - protein, 48 aa, Homo sapiens
VGQDTQEVIVVPHSLPFKVVVISAILALVVLTIISLIILIMLWQKKPR

SEQ ID NO: 26 - DNA, 147 bp, Homo sapiens
gctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 27 - protein, 49 aa, Homo sapiens
AVGQDTQEVIVVPHSLPFKVVVISAILALVVLTIISLIILIMLWQKKPR

SEQ ID NO: 28 - DNA, 66 bp, Artificial Sequence
  P2A sequence
ggatctggggccaccaacttttcattgctcaagcaggcgggcgatgtggaggaaaaccctggcccc

SEQ ID NO: 29 - protein, 22 aa, Artificial Sequence
  P2A sequence
GSGATNFSLLKQAGDVEENPGP

SEQ ID NO: 30 - DNA, 705 bp, Mus musculus
gtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattcttgacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcc

SEQ ID NO: 31 - protein, 238 aa, Mus musculus
VSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAAAA

SEQ ID NO: 32 - DNA, 1002 bp, Mus musculus
tgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttcttgatgctggccagtacacctgccacaaaggaggcgagactctgagccactcacatctgctgctccacaagaaggaaaatggaatttggtccactgaaattttaaagaatttcaagaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcc

SEQ ID NO: 33 - protein, 333 aa, Mus musculus
CPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVR

SEQ ID NO: 34 - DNA, 375 bp, Mus musculus
aagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactaca

SEQ ID NO: 35 - protein, 125 aa, Mus musculus
KSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTT

SEQ ID NO: 36 - DNA, 360 bp, Artificial Sequence
  anti-CTLA4 9D9 variable light chain sequence
gacattgtgatgacacagaccacactcagtctccccgtttcccttggtgatcaagcctccatatcctgtaggtctagtcaatctatcgtccactccaacggcaatacctatctggaatggtatcttcaaaagcccggacaatcaccaaagcttcttatctataaggtgagcaatagatttagcggggtccctgaccgattctctggaagtggctctggcacagactttaccttgaaaatctccagagttgaggctgaggaccttggtgtatactactgcttccaaggctctcatgttccctacactttcggaggcggaacaaaactggagataaaacgagccgacgcagcccccactgtg

SEQ ID NO: 37 - protein, 120 aa, Artificial Sequence
  anti-CTLA4 9D9 variable light chain sequence
DIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTV

SEQ ID NO: 38 - DNA, 390 bp, Artificial Sequence
  anti-CTLA4 9D9 variable heavy chain sequence
gaggcaaagcttcaggaatctggtccagtgttggtgaaaccaggtgcatccgtgaaaatgtcctgcaaagcaagcggttacacttttactgactattatatgaactgggtaaagcaatcccacggcaaatccctggaatggattggtgtcatcaacccttacaacggtgatacaagttacaaccaaaagttcaaaggtaaggctacattgaccgtagataagagtagcagtactgcatacatggaacttaactctcttacatccgaggactccgctgtttactattgtgcacgctactacgggagctggttcgcttactggggtcaaggcaccctgataacagtgtccacagccaaaaccacacctccctccgtctatcctctcgctcca

SEQ ID NO: 39 - protein, 130 aa, Artificial Sequence
  anti-CTLA4 9D9 variable heavy chain sequence
EAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAP

SEQ ID NO: 40 - DNA, 333 bp, Artificial Sequence
  anti-CTLA4 9H10 variable light chain sequence
gacattgtgatgacacagagtccttcatcccttgcagtcagtgtcggcgaaaaagtaacaatttcatgcaagtctagtcaatctctgttgtacggctcctctcattacctcgcatggtatcaacaaaaagtgggtcaatctcccaaattgttgatatactgggcttcaactagacacactggaatccctgacaggttcattggtagcggatcagggactgactttacactgtccctcagcagcgtacaagcagaagacatggccgactatttctgccaacaatactttagtacaccatggacctttggggctgggaccagagttgagataaaa

SEQ ID NO: 41 - protein, 111 aa, Artificial Sequence
  anti-CTLA4 9H10 variable light chain sequence
DIVMTQSPSSLAVSVGEKVTISCKSSQSLLYGSSHYLAWYQQKVGQSPKLLIYWASTRHTGIPDRFIGSGSGTDFTLSLSSVQAEDMADYFCQQYFSTPWTFGAGTRVEIK

SEQ ID NO: 42 - DNA, 384 bp, Artificial Sequence
  anti-CTLA4 9H10 variable heavy acid sequence
caagtgcagctgcttcaatccgaatcagaactcgtgaagccaggcgcttcagtgaaattgtcttgtaagacttcaggatacactttcactgattactatatacactgggttaagcagaagcctggtcagggtcttgaatggattggcctcatcaatcccaataacgatggcacaaactacaaccagaaatttcaaggaaaagccacacttaccgcagacaaatccagttctaccgcatacatggaacttaatagtctcacttttgatgactcagtaatatatttctgtgccagggccagtagccgacttagaatggctaggactacctctgactactatgccatggactattggggacagggcattcaagtgaccgtgagctct

SEQ ID NO: 43 - protein, 128 aa, Artificial Sequence
  anti-CTLA4 9H10 variable heavy sequence
QVQLLQSESELVKPGASVKLSCKTSGYTFTDYYIHWVKQKPGQGLEWIGLINPNNDGTNYNQKFQGKATLTADKSSSTAYMELNSLTFDDSVIYFCARASSRLRMARTTSDYYAMDYWGQGIQVTVSS

SEQ ID NO: 44 - DNA, 669 bp, Mus musculus
ggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 45 - protein, 222 aa, Mus musculus
GCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 46 - DNA, 357 bp, Artificial Sequence
  OKT3 Variable heavy chain nucleic acid sequence
caggtgcagctgcagcaatctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccacactcaccgtctcctca

SEQ ID NO: 47 - protein, 119 aa, Artificial Sequence [sequence omitted]

SEQ ID NO: 48 - DNA, 321 bp, Artificial Sequence
  OKT3 Variable light chain sequence
cagattgtgctcacccagtctccagcaatcatgtctgcatctccaggggagaaggttaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgt

SEQ ID NO: 49 - DNA, 321 bp, Artificial Sequence
  OKT3 Variable light chain sequence
cagattgtgctcacccagtctccagcaatcatgtctgcatctccaggggagaaggttaccatgacctgcagtgccagctcaagtgtaagttacatgaactggtatcagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgt

SEQ ID NO: 50 - protein, 107 aa, Homo sapiens
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 51 - DNA, 756 bp, Homo sapiens
tggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcc

SEQ ID NO: 52 - DNA, 756 bp, Homo sapiens
tggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctcgaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggaccctaagaggcaaatcttcctagatcaaaacatgctggcagttattgatgagctgatgcaggccctgaatttcaacagtgagactgtgccacaaaaatcctcccttgaagaaccggatttctacaagactaaaatcaagctctgcatacttcttcatgctttcagaatccgggcagtgactattgatagagtgatgagctatctgaatgcttcc

SEQ ID NO: 53 - protein, 252 aa, Homo sapiens
WPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 54 - DNA, 984 bp, Homo sapiens
tgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 55 - DNA, 984 bp, Homo sapiens
tgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaactctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttcg

SEQ ID NO: 56 - protein, 327 aa, Homo sapiens
CHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 57 - DNA, 375 bp, Homo sapiens
aagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 58 - protein, 124 aa, Homo sapiens
KKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 59 - DNA, 1011 bp, Artificial Sequence
  Igkappa-HA-2C11VHC-Linker-C211VLC-Myc-PDGFR nucleic acid sequence
atggagacagacacactcctgctatgggtactgctgctctgggttccaggttccactggtgactatccatatgatgttccagattatgctggggcccagccggccagatctgaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggcggtggctccggcggtggtgggtcgggtggcggcggatctgacatccagatgacccagtctccatcatcactgcctgcctccctgggagacagagtcactatcaattgtcaggccagtcaggacattagcaattatttaaactggtaccagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaagtcgacgaacaaaaactcatctcagaagaggatctgtacactgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 60 - protein, 337 aa, Artificial Sequence
  Igkappa-HA-2C11VHC-Linker-C211VLC-Myc-PDGFR amino acid sequence
METDTLLLWVLLLWVPGSTGDYPYDVPDYAGAQPARSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKVDEQKLISEEDLYTVGQDTQEVIVVPHSLPFKVVVISAILALVVLTIISLIILIMLWQKKPR

SEQ ID NO: 61 - DNA, 1002 bp, Artificial Sequence
  Igkappa-HA-2C11VHC-Linker-2C11VLC-Linker-PDGFR sequence
atggagacagacacactcctgctatgggtactgctgctctgggttccaggttccactggtgactatccatatgatgttccagattatgctggggcccagccggccagatctgaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggcggtggctccggcggtggtgggtcgggtggcggcggatctgacatccagatgacccagtctccatcatcactgcctgcctccctgggagacagagtcactatcaattgtcaggccagtcaggacattagcaattatttaaactggtatcagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaaggcagtgggagtgggagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 62 - protein, 334 aa, Artificial Sequence
  Igkappa-HA-2C11VHC-Linker-2C11VLC-Linker-PDGFR sequence
METDTLLLWVLLLWVPGSTGDYPYDVPDYAGAQPARSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGSGSGSGSGNAVGQDTQEVIVVPHSLPFKVVVISAILALVVLTIISLIILIMLWQKKPR

SEQ ID NO: 63 - DNA, 2853 bp, Artificial Sequence
  Igkappa-HA-2C11VHC-Linker-2C11VLC-Linker-PDGFR- P2A-mIL-12p35-P2A-mIL-12p40 sequence
atggagacagacacactcctgctatgggtactgctgctctgggttccaggttccactggtgactatccatatgatgttccagattatgctggggcccagccggccagatctgaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggcggtggctccggcggtggtgggtcgggtggcggcggatctgacatccagatgacccagtctccatcatcactgcctgcctccctgggagacagagtcactatcaattgtcaggccagtcaggacattagcaattatttaaactggtatcagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaaggcagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatctggggccaccaacttttcattgctcaagcaggcgggcgatgtggaggaaaaccctggccccggtaccgtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattcttgacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgcggccgcaggatctggggccaccaacttttcattgctcaagcaggcgggcgatgtggaggaaaaccctggccccggatcctgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttcttgatgctggccagtacacctgccacaaaggaggcgagactctgagccactcacatctgctgctccacaagaaggaaaatggaatttggtccactgaaattttaaagaatttcaagaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 64 - protein, 950 aa, Artificial Sequence
  Igkappa-HA-2C11VHC-Linker-2C11VLC-Linker-PDGFR- P2A-mIL-12p35-P2A-mIL-12p40 sequence
METDTLLLWVLLLWVPGSTGDYPYDVPDYAGAQPARSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGSGSGNAVGQDTQEVIVVPHSLPFKVVVISAILALVVLTIISLIILIMLWQKKPRGSGATNFSLLKQAGDVEENPGPGTVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAAAAGSGATNFSLLKQAGDVEENPGPGSCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 65 - DNA, 2826 bp, Artificial Sequence
  Igkappa-2C11VHC-Linker-2C11VLC-Linker-PDGFR- P2A-mIL-12p35-P2A-mIL-12p40 sequence
atggagacagacacactcctgctatgggtactgctgctctgggttccaggttccactggtgacggggcccagccggccagatctgaggtgcagctggtggagtctgggggaggcttggtgcagcctggaaagtccctgaaactctcctgtgaggcctctggattcaccttcagcggctatggcatgcactgggtccgccaggctccagggagggggctggagtcggtcgcatacattactagtagtagtattaatatcaaatatgctgacgctgtgaaaggccggttcaccgtctccagagacaatgccaagaacttactgtttctacaaatgaacattctcaagtctgaggacacagccatgtactactgtgcaagattcgactgggacaaaaattactggggccaaggaaccatggtcaccgtctcctcaggtggcggtggctccggcggtggtgggtcgggtggcggcggatctgacatccagatgacccagtctccatcatcactgcctgcctccctgggagacagagtcactatcaattgtcaggccagtcaggacattagcaattatttaaactggtatcagcagaaaccagggaaagctcctaagctcctgatctattatacaaataaattggcagatggagtcccatcaaggttcagtggcagtggttctgggagagattcttctttcactatcagcagcctggaatccgaagatattggatcttattactgtcaacagtattataactatccgtggacgttcggacctggcaccaagctggaaatcaaaggcagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatctggggccaccaacttttcattgctcaagcaggcgggcgatgtggaggaaaaccctggccccggtaccgtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattcttgacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgcggccgcaggatctggggccaccaacttttcattgctcaagcaggcgggcgatgtggaggaaaaccctggccccggatcctgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttcttgatgctggccagtacacctgccacaaaggaggcgagactctgagccactcacatctgctgctccacaagaaggaaaatggaatttggtccactgaaattttaaagaatttcaagaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctag

SEQ ID NO: 66 - protein, 941 aa, Artificial Sequence
  Igkappa-2C11VHC-Linker-2C11VLC-Linker-PDGFR- P2A-mIL-12p35-P2A-mIL-12p40 sequence
METDTLLLWVLLLWVPGSTGDGAQPARSEVQLVESGGGLVQPGKSLKLSCEASGFTFSGYGMHWVRQAPGRGLESVAYITSSSINIKYADAVKGRFTVSRDNAKNLLFLQMNILKSEDTAMYYCARFDWDKNYWGQGTMVTVSSGGGGSGGGGSGGGGSDIQMTQSPSSLPASLGDRVTINCQASQDISNYLNWYQQKPGKAPKLLIYYTNKLADGVPSRFSGSGSGRDSSFTISSLESEDIGSYYCQQYYNYPWTFGPGTKLEIKGSGSGNAVGQDTQEVIVVPHSLPFKVVVISAILALVVLTIISLIILIMLWQKKPRGSGATNFSLLKQAGDVEENPGPGTVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAAAAGSGATNFSLLKQAGDVEENPGPGSCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 67 - DNA, 2244 bp, Artificial Sequence
  mIL-12 p35-P2A-mIL-12 p40-P2A-mCXCL9 sequence
atggtcagcgttccaacagcctcaccctcggcatccagcagctcctctcagtgccggtccagcatgtgtcaatcacgctacctcctctttttggccacccttgccctcctaaaccacctcagtttggccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattcttgacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccgcggccgcaggatctggggccaccaacttttcattgctcaagcaggcgggcgatgtggaggaaaaccctggccccggatcctgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttcttgatgctggccagtacacctgccacaaaggaggcgagactctgagccactcacatctgctgctccacaagaaggaaaatggaatttggtccactgaaattttaaagaatttcaagaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatcctcgtctagaggatctggggccaccaacttttcattgctcaagcaggcgggcgatgtggaggaaaaccctggccccaagtccgctgttcttttcctcttgggcatcatcttcctggagcagtgtggagttcgaggaaccctagtgataaggaatgcacgatgctcctgcatcagcaccagccgaggcacgatccactacaaatccctcaaagacctcaaacagtttgccccaagccccaattgcaacaaaactgaaatcattgctacactgaagaacggagatcaaacctgcctagatccggactcggcaaatgtgaagaagctgatgaaagaatgggaaaagaagatcagccaaaagaaaaagcaaaagagggggaaaaaacatcaaaagaacatgaaaaacagaaaacccaaaacaccccaaagtcgtcgtcgttcaaggaagactacataa

SEQ ID NO: 68 - protein, 747 aa, Artificial Sequence
  mIL-12 p35-P2A-mIL-12 p40-P2A-mCXCL9 sequence
MVSVPTASPSASSSSSQCRSSMCQSRYLLFLATLALLNHLSLARVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAAAAGSGATNFSLLKQAGDVEENPGPGSCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSSSRGSGATNFSLLKQAGDVEENPGPKSAVLFLLGIIFLEQCGVRGTLVIRNARCSCISTSRGTIHYKSLKDLKQFAPSPNCNKTEIIATLKNGDQTCLDPDSANVKKLMKEWEKKISQKKKQKRGKKHQKNMKNRKPKTPQSRRRSRKTT

SEQ ID NO: 69 - DNA, 1596 bp, Artificial Sequence
  Igkappa-HA-9D9 VLC-Linker-9D9 VHC-Linker-mIgG1 Fc domain (anti-CTLA4 scFv) sequence
atggagacagacacactcctgctatgggtactgctgctctgggttccaggttccactggtgactatccatatgatgttccagattatgctggggcccagccggccagatctgacattgtgatgacacagaccacactcagtctccccgtttcccttggtgatcaagcctccatatcctgtaggtctagtcaatctatcgtccactccaacggcaatacctatctggaatggtatcttcaaaagcccggacaatcaccaaagcttcttatctataaggtgagcaatagatttagcggggtccctgaccgattctctggaagtggctctggcacagactttaccttgaaaatctccagagttgaggctgaggaccttggtgtatactactgcttccaaggctctcatgttccctacactttcggaggcggaacaaaactggagataaaacgagccgacgcagcccccactgtgagtggctctggagggggctctggcggtggatctgggggtggaagtgaggcaaagcttcaggaatctggtccagtgttggtgaaaccaggtgcatccgtgaaaatgtcctgcaaagcaagcggttacacttttactgactattatatgaactgggtaaagcaatcccacggcaaatccctggaatggattggtgtcatcaacccttacaacggtgatacaagttacaaccaaaagttcaaaggtaaggctacattgaccgtagataagagtagcagtactgcatacatggaacttaactctcttacatccgaggactccgctgtttactattgtgcacgctactacgggagctggttcgcttactggggtcaaggcaccctgataacagtgtccacagccaaaaccacacctccctccgtctatcctctcgctccagtcgactctagtggatccggtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 70 - protein, 531 aa, Artificial Sequence
  Igkappa-HA-9D9 VLC-Linker-9D9 VHC-Linker-mIgG1 Fc domain (anti-CTLA4 scFv) sequence
METDTLLLWVLLLWVPGSTGDYPYDVPDYAGAQPARSDIVMTQTTLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIKRADAAPTVSGSGGGSGGGSGGGSEAKLQESGPVLVKPGASVKMSCKASGYTFTDYYMNWVKQSHGKSLEWIGVINPYNGDTSYNQKFKGKATLTVDKSSSTAYMELNSLTSEDSAVYYCARYYGSWFAYWGQGTLITVSTAKTTPPSVYPLAPVDSSGSGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 71 - DNA, 1563 bp, Artificial Sequence
  Igkappa-HA-9H10 VLC-Linker-9H10 VHC-Linker- mIgG1 Fc domain (anti-CTLA4 scFv) sequence
atggagacagacacactcctgctatgggtactgctgctctgggttccaggttccactggtgactatccatatgatgttccagattatgctggggcccagccggccagatctgacattgtgatgacacagagtccttcatcccttgcagtcagtgtcggcgaaaaagtaacaatttcatgcaagtctagtcaatctctgttgtacggctcctctcattacctcgcatggtatcaacaaaaagtgggtcaatctcccaaattgttgatatactgggcttcaactagacacactggaatccctgacaggttcattggtagcggatcagggactgactttacactgtccctcagcagcgtacaagcagaagacatggccgactatttctgccaacaatactttagtacaccatggacctttggggctgggaccagagttgagataaaaagtggctctggagggggctctggcggtggatctgggggtggaagtcaagtgcagctgcttcaatccgaatcagaactcgtgaagccaggcgcttcagtgaaattgtcttgtaagacttcaggatacactttcactgattactatatacactgggttaagcagaagcctggtcagggtcttgaatggattggcctcatcaatcccaataacgatggcacaaactacaaccagaaatttcaaggaaaagccacacttaccgcagacaaatccagttctaccgcatacatggaacttaatagtctcacttttgatgactcagtaatatatttctgtgccagggccagtagccgacttagaatggctaggactacctctgactactatgccatggactattggggacagggcattcaagtgaccgtgagctctgtcgactctagtggatccggtggttgtaagccttgcatatgtacagtcccagaagtatcatctgtcttcatcttccccccaaagcccaaggatgtgctcaccattactctgactcctaaggtcacgtgtgttgtggtagacatcagcaaggatgatcccgaggtccagttcagctggtttgtagatgatgtggaggtgcacacagctcagacgcaaccccgggaggagcagttcaacagcactttccgctcagtcagtgaacttcccatcatgcaccaggactggctcaatggcaaggagttcaaatgcagggtcaacagtgcagctttccctgcccccatcgagaaaaccatctccaaaaccaaaggcagaccgaaggctccacaggtgtacaccattccacctcccaaggagcagatggccaaggataaagtcagtctgacctgcatgataacagacttcttccctgaagacattactgtggagtggcagtggaatgggcagccagcggagaactacaagaacactcagcccatcatggacacagatggctcttacttcgtctacagcaagctcaatgtgcagaagagcaactgggaggcaggaaatactttcacctgctctgtgttacatgagggcctgcacaaccaccatactgagaagagcctctcccactctcctggtaaatga

SEQ ID NO: 72 - protein, 520 aa, Artificial Sequence
  Igkappa-HA-9H10 VLC-Linker-9H10 VHC-Linker- mIgG1 Fc domain (anti-CTLA4 scFv) sequence
METDTLLLWVLLLWVPGSTGDYPYDVPDYAGAQPARSDIVMTQSPSSLAVSVGEKVTISCKSSQSLLYGSSHYLAWYQQKVGQSPKLLIYWASTRHTGIPDRFIGSGSGTDFTLSLSSVQAEDMADYFCQQYFSTPWTFGAGTRVEIKSGSGGGSGGGSGGGSQVQLLQSESELVKPGASVKLSCKTSGYTFTDYYIHWVKQKPGQGLEWIGLINPNNDGTNYNQKFQGKATLTADKSSSTAYMELNSLTFDDSVIYFCARASSRLRMARTTSDYYAMDYWGQGIQVTVSSVDSSGSGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 73 - DNA, 1020 bp, Artificial Sequence
  Igkappa-HA-OKT3 VHC-Linker-OKT3 VLC-Myc-PDGFR sequence
atggagacagacacactcctgctatgggtactgctgctctgggttccaggttccactggtgactatccatatgatgttccagattatgctggggcccagccggccagatctcaggtgcagctgcagcaatctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccacactcaccgtctcctcaggtggcggtggctccggcggtggtgggtcgggtggcggcggatctcagattgtgctcacccagtctccagcaatcatgtctgcatctccaggggagaaggttaccatgacctgcagtgccagctcaagtgtaagttacatgaactggtaccagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgtgtcgacgaacaaaaactcatctcagaagaggatctgaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 74 - protein, 340 aa, Artificial Sequence
  Igkappa-HA-OKT3 VHC-Linker-OKT3 VLC-Myc-PDGFR sequence
METDTLLLWVLLLWVPGSTGDYPYDVPDYAGAQPARSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRVDEQKLISEEDLNAVGQDTQEVIVVPHSLPFKVVVISAILALVVLTIISLIILIMLWQKKPR

SEQ ID NO: 75 - DNA, 1011 bp, Artificial Sequence
  Igkappa-HA-OKT3 VHC-Linker-OKT3 VLC-Linker- PDGFR sequence
atggagacagacacactcctgctatgggtactgctgctctgggttccaggttccactggtgactatccatatgatgttccagattatgctggggcccagccggccagatctcaggtgcagctgcagcaatctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccacactcaccgtctcctcaggtggcggtggctccggcggtggtgggtcgggtggcggcggatctcagattgtgctcacccagtctccagcaatcatgtctgcatctccaggggagaaggttaccatgacctgcagtgccagctcaagtgtaagttacatgaactggtatcagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgtggcagtgggagtgggagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgt

SEQ ID NO: 76 - protein, 337 aa, Artificial Sequence
  Igkappa-HA-OKT3 VHC-Linker-OKT3 VLC-Linker- PDGFR sequence
METDTLLLWVLLLWVPGSTGDYPYDVPDYAGAQPARSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSGSGSGSGNAVGQDTQEVIVVPHSLPFKVVVISAILALVVLTIISLIILIMLWQKKPR

SEQ ID NO: 77 - DNA, 2877 bp, Artificial Sequence
  Igkappa-HA-OKT3 VHC-Linker-OKT3 VLC-Linker- PDGFR-P2A-hIL-12 p35-P2A-hIL-12 p40 sequence
atggagacagacacactcctgctatgggtactgctgctctgggttccaggttccactggtgactatccatatgatgttccagattatgctggggcccagccggccagatctcaggtgcagctgcagcaatctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccacactcaccgtctcctcaggtggcggtggctccggcggtggtgggtcgggtggcggcggatctcagattgtgctcacccagtctccagcaatcatgtctgcatctccaggggagaaggttaccatgacctgcagtgccagctcaagtgtaagttacatgaactggtatcagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgtggcagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatctggggccaccaacttttcattgctcaagcaggcgggcgatgtggaggaaaaccctggccccggtacctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggatctggggccaccaacttttcattgctcaagcaggcgggcgatgtggaggaaaaccctggcccctgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 78 - protein, 958 aa, Artificial Sequence
  Igkappa-HA-OKT3 VHC-Linker-OKT3 VLC-Linker- PDGFR-P2A-hIL-12 p35-P2A-hIL-12 p40 sequence
METDTLLLWVLLLWVPGSTGDYPYDVPDYAGAQPARSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSGSGNAVGQDTQEVIVVPHSLPFKVVVISAILALVVLTIISLIILIMLWQKKPRGSGATNFSLLKQAGDVEENPGPGTWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 79 - DNA, 2850 bp, Artificial Sequence
  Igkappa-OKT3 VHC-Linker-OKT3 VLC-Linker-PDGFR- P2A-hIL-12 p35-P2A-hIL-12 p40 sequence
atggagacagacacactcctgctatgggtactgctgctctgggttccaggttccactggtgacggggcccagccggccagatctcaggtgcagctgcagcaatctggggctgaactggcaagacctggggcctcagtgaagatgtcctgcaaggcttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccacactcaccgtctcctcaggtggcggtggctccggcggtggtgggtcgggtggcggcggatctcagattgtgctcacccagtctccagcaatcatgtctgcatctccaggggagaaggttaccatgacctgcagtgccagctcaagtgtaagttacatgaactggtatcagcagaagtcaggcacctcccccaaaagatggatttatgacacatccaaactggcttctggagtccctgctcacttcaggggcagtgggtctgggacctcttactctctcacaatcagcggcatggaggctgaagatgctgccacttattactgccagcagtggagtagtaacccattcacgttcggctcggggaccaagctggagatcaatcgtggcagtgggagtgggaatgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgtggatctggggccaccaacttttcattgctcaagcaggcgggcgatgtggaggaaaaccctggccccggtacctggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggatctggggccaccaacttttcattgctcaagcaggcgggcgatgtggaggaaaaccctggcccctgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 80 - protein, 949 aa, Artificial Sequence
  Igkappa-OKT3 VHC-Linker-OKT3 VLC-Linker-PDGFR- P2A-hIL-12 p35-P2A-hIL-12 p40 sequence
METDTLLLWVLLLWVPGSTGDGAQPARSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSGSGNAVGQDTQEVIVVPHSLPFKVVVISAILALVVLTIISLIILIMLWQKKPRGSGATNFSLLKQAGDVEENPGPGTWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASGSGATNFSLLKQAGDVEENPGPCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 81 - DNA, 2271 bp, Artificial Sequence
  hIL-12 p35-P2A-hIL-12p40-P2A-hCXCL9 sequence
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctcgaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggaccctaagaggcaaatcttcctagatcaaaacatgctggcagttattgatgagctgatgcaggccctgaatttcaacagtgagactgtgccacaaaaatcctcccttgaagaaccggatttctacaagactaaaatcaagctctgcatacttcttcatgctttcagaatccgggcagtgactattgatagagtgatgagctatctgaatgcttccgcggccgcaggatctggggccaccaacttttcattgctcaagcaggccggcgatgtggaggaaaaccctggccccggatcctgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaactctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttcgtctagaggatctggggccaccaacttttcattgctcaagcaggcgggcgatgtggaggaaaaccctggccccaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 82 - protein, 756 aa, Artificial Sequence
  hIL-12 p35-P2A-hIL-12p40-P2A-hCXCL9 sequence
MWPPGSASQPPPSPAAATGLHPAARPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASAAAGSGATNFSLLKQAGDVEENPGPGSCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSSRGSGATNFSLLKQAGDVEENPGPKKSGVLFLLGIILLVLIGVQGTPVVRKGRCSCISTNQGTIHLQSLKDLKQFAPSPSCEKIEIIATLKNGVQTCLNPDSADVKELIKKWEKQVSQKKKQKNGKKHQKKKVLKVRKSQRSRQKKTT

SEQ ID NO: 83 - protein, 21 aa, Mus musculus
VVISAILALVVLTVISLIILI

SEQ ID NO: 84 - protein, 21 aa, Homo sapiens
VVISAILALVVLTIISLIILI

SEQ ID NO: 85 - protein, 21 aa, Homo sapiens
AAVLVLLVIVIISLIVLVVIW

SEQ ID NO: 86 - protein, 21 aa, Mus musculus
AAVLVLLVIVIVSLIVLVVIW

SEQ ID NO: 87 - DNA, 62 bp, Homo sapiens
tggtgatctcagccatcctggccctggtggtgctcaccatcatctcccttatcatcctcatc

SEQ ID NO: 88 - DNA, 63 bp, Homo sapiens
gtggtgatctcagccatcctggccctggtggtgctcaccatcatctcccttatcatcctcatc

SEQ ID NO: 89 - DNA, 65 bp, Homo sapiens
gctgcagtcctggtgctgttggtgattgtgatcatctcacttattgtcctggttgtcatttggaa